SEQUENCE LISTING (81 sequences)

SEQ ID NO: 1 - DNA, 2912 bp, Sorghum bicolor
ttgcccgtcagtgagtcgtattacaccgggtggatggcccggccgacgcgtccgatctgtcccagttctctgttctgttctgtcgacgccattcctgtgctctgccgtcccagcgtttgccaagtattgagtgtcattgagccatggctgccaagttgactcgcctccacagtcttcgcgaacgccttggtgccaccttctcctctcatcccaatgagctgattgcactcttctccaggtatgttaaccagggcaagggaatgcttcagcgccatcaactgcttgctgagtttgatgccctgtttgatagtgacaaggagaagtatgcgcccttcgaagactttcttcgtgctgctcaggaagcaattgtgctccctccctgggtagcacttgctatcaggccaaggcctggtgtctgggattacattcgagtgaatgtaagcgagttggctgtggaggagctgagtgtttctgagtacttggcattcaaggaacagctggtggatggaaattccaacagcaactttgttcttgagcttgattttgagcccttcaatgcctcattccctcgtccttccatgtcaaagtccattggaaatggagtgcaattccttaaccgacacctgtcttccaagttgttccaggacaaggagagcctgtacccattgctgaatttcctcaaagcccataactacaagggcacgacgatgatgttgaatgacagaattcagagcctccgtgggctccagtcatcccttagaaaggcagaagagtatctactgagtgtccctcaagacactccctactcagagttcaaccataggttccaagagcttggcttggagaagggttggggtgacactgcaaagcgcgtacttgatacactccacttgcttcttgaccttcttgaggcccctgatcctgccaacttggagaagttccttggaactataccaatgatgttcaatgttgttatcctgtctcctcatggctactttgcccaatccaatgtgcttggataccctgacactggtggtcaggttgtgtacattttggatcaagtccgtgctttggagaatgagatgcttcttaggattaagcagcaaggccttgacatcaccccgaagatcctcattgttaccaggctgttgcctgatgctgttgggactacgtgcggtcagcgtctggagaaggtcattggaaccgagcacacagacattattcgtattccattcagaaatgagaatggtattctccgcaagtggatctctcgttttgatgtctggccatacctggagacatacactgaggatgttgccagtgaaataatgttagaaatgcaggccaagcctgaccttattgttggcaactacagtgatggcaatctagtcgccactctgctcgcgcacaagttgggagttactcagtgtaccattgcccacgccttggagaaaaccaaatatcccaactcagacatatacttagacaaatttgacagccaataccacttctcatgccagttcacagctgaccttattgccatgaatcacactgatttcatcatcaccagtacattccaagaaatcgcgggaagcaaggacactgtggggcagtatgagtcccacattgcgttcactcttcctggactttaccgtgttgtccatggcattgatgtttttgatcccaaattcaacattgtctctcctggagcagacatgagtgtttactacccatacactgaaactgacaagagactcactgccttccatcctgaaattgaggagctcatctacagtgatgttgagaatgatgagcacaagtttgtgttgaaggacaagaacaagccgatcatcttctcaatggctcgtcttgaccgtgtgaagaacatgacaggcttggttgagatgtatggtaagaatgcacgcctgagggaattggcaaaccttgtgattgttgctggtgaccatggcaaggaatcgaaggacagggaggagcaggcagagttcaagaagatgtacagtctcattgatgagtacaacttgaagggccatatccggtggatctcagctcagatgaaccgtgtccgcaacgctgagttgtaccgctacatttgtgacacgaagggagcatttgtgcagcctgcattctatgaagcattcggcctgactgtcattgagtccatgacgtgcggtttgccaacaattgcaacctgccatggtggccctgctgaaataattgtggacggggtgtctggtttgcacattgatccttaccacagtgacaaggctgcagatattttggtcaacttctttgagaagtgcaaggcagacccaagctactgggacaagatctcacagggtggactgcagagaatttatgagaagtacacctggaagctctactccgagaggctgatgaccctgactggtgtatacggattctggaagtatgtgagcaatctggagaggcgtgagactcgccgctaccttgagatgttctatgctctgaaataccgtagcctggcaagtgcggttccattgtccttcgattagtgtgggaaagaagaaccccaatctggagtagtggagaaccatcatctgcatttcgattgttcgctgcaattcgcattgttagttgtgtatttgagttatgtgtacttggtttccaagcactttggttcctttttgcgagttttgggcagcgctggctggttccttttataggaattagctgcaccttttgcttcaaataaacgcctgctcgttcacctgtcttccaaagttcaatgcaatgttttgttgcccaagtcttcatttctgactgatggtgatgttatgttctgtcagttctgttaatcacctgtttaatgtggtaggctgatgcctgttcttattatcaaaggttgctgtgcc

SEQ ID NO: 2 - protein, 802 aa, Sorghum bicolor
MAAKLTRLHSLRERLGATFSSHPNELIALFSRYVNQGKGMLQRHQLLAEFDALFDSDKEKYAPFEDFLRAAQEAIVLPPWVALAIRPRPGVWDYIRVNVSELAVEELSVSEYLAFKEQLVDGNSNSNFVLELDFEPFNASFPRPSMSKSIGNGVQFLNRHLSSKLFQDKESLYPLLNFLKAHNYKGTTMMLNDRIQSLRGLQSSLRKAEEYLLSVPQDTPYSEFNHRFQELGLEKGWGDTAKRVLDTLHLLLDLLEAPDPANLEKFLGTIPMMFNVVILSPHGYFAQSNVLGYPDTGGQVVYILDQVRALENEMLLRIKQQGLDITPKILIVTRLLPDAVGTTCGQRLEKVIGTEHTDIIRIPFRNENGILRKWISRFDVWPYLETYTEDVASEIMLEMQAKPDLIVGNYSDGNLVATLLAHKLGVTQCTIAHALEKTKYPNSDIYLDKFDSQYHFSCQFTADLIAMNHTDFIITSTFQEIAGSKDTVGQYESHIAFTLPGLYRVVHGIDVFDPKFNIVSPGADMSVYYPYTETDKRLTAFHPEIEELIYSDVENDEHKFVLKDKNKPIIFSMARLDRVKNMTGLVEMYGKNARLRELANLVIVAGDHGKESKDREEQAEFKKMYSLIDEYNLKGHIRWISAQMNRVRNAELYRYICDTKGAFVQPAFYEAFGLTVIESMTCGLPTIATCHGGPAEIIVDGVSGLHIDPYHSDKAADILVNFFEKCKADPSYWDKISQGGLQRIYEKYTWKLYSERLMTLTGVYGFWKYVSNLERRETRRYLEMFYALKYRSLASAVPLSFD

SEQ ID NO: 3 - DNA, 2409 bp, Sorghum bicolor
atggctgccaagttgactcgcctccacagtcttcgcgaacgccttggtgccaccttctcctctcatcccaatgagctgattgcactcttctccaggtatgttaaccagggcaagggaatgcttcagcgccatcaactgcttgctgagtttgatgccctgtttgatagtgacaaggagaagtatgcgcccttcgaagactttcttcgtgctgctcaggaagcaattgtgctccctccctgggtagcacttgctatcaggccaaggcctggtgtctgggattacattcgagtgaatgtaagcgagttggctgtggaggagctgagtgtttctgagtacttggcattcaaggaacagctggtggatggaaattccaacagcaactttgttcttgagcttgattttgagcccttcaatgcctcattccctcgtccttccatgtcaaagtccattggaaatggagtgcaattccttaaccgacacctgtcttccaagttgttccaggacaaggagagcctgtacccattgctgaatttcctcaaagcccataactacaagggcacgacgatgatgttgaatgacagaattcagagcctccgtgggctccagtcatcccttagaaaggcagaagagtatctactgagtgtccctcaagacactccctactcagagttcaaccataggttccaagagcttggcttggagaagggttggggtgacactgcaaagcgcgtacttgatacactccacttgcttcttgaccttcttgaggcccctgatcctgccaacttggagaagttccttggaactataccaatgatgttcaatgttgttatcctgtctcctcatggctactttgcccaatccaatgtgcttggataccctgacactggtggtcaggttgtgtacattttggatcaagtccgtgctttggagaatgagatgcttcttaggattaagcagcaaggccttgacatcaccccgaagatcctcattgttaccaggctgttgcctgatgctgttgggactacgtgcggtcagcgtctggagaaggtcattggaaccgagcacacagacattattcgtattccattcagaaatgagaatggtattctccgcaagtggatctctcgttttgatgtctggccatacctggagacatacactgaggatgttgccagtgaaataatgttagaaatgcaggccaagcctgaccttattgttggcaactacagtgatggcaatctagtcgccactctgctcgcgcacaagttgggagttactcagtgtaccattgcccacgccttggagaaaaccaaatatcccaactcagacatatacttagacaaatttgacagccaataccacttctcatgccagttcacagctgaccttattgccatgaatcacactgatttcatcatcaccagtacattccaagaaatcgcgggaagcaaggacactgtggggcagtatgagtcccacattgcgttcactcttcctggactttaccgtgttgtccatggcattgatgtttttgatcccaaattcaacattgtctctcctggagcagacatgagtgtttactacccatacactgaaactgacaagagactcactgccttccatcctgaaattgaggagctcatctacagtgatgttgagaatgatgagcacaagtttgtgttgaaggacaagaacaagccgatcatcttctcaatggctcgtcttgaccgtgtgaagaacatgacaggcttggttgagatgtatggtaagaatgcacgcctgagggaattggcaaaccttgtgattgttgctggtgaccatggcaaggaatcgaaggacagggaggagcaggcagagttcaagaagatgtacagtctcattgatgagtacaacttgaagggccatatccggtggatctcagctcagatgaaccgtgtccgcaacgctgagttgtaccgctacatttgtgacacgaagggagcatttgtgcagcctgcattctatgaagcattcggcctgactgtcattgagtccatgacgtgcggtttgccaacaattgcaacctgccatggtggccctgctgaaataattgtggacggggtgtctggtttgcacattgatccttaccacagtgacaaggctgcagatattttggtcaacttctttgagaagtgcaaggcagacccaagctactgggacaagatctcacagggtggactgcagagaatttatgagaagtacacctggaagctctactccgagaggctgatgaccctgactggtgtatacggattctggaagtatgtgagcaatctggagaggcgtgagactcgccgctaccttgagatgttctatgctctgaaataccgtagcctggcaagtgcggttccattgtccttcgattag

SEQ ID NO: 4 - DNA, 19 bp, Artificial Sequence
  SoSUS1 F
tggtccggctgagatcatc

SEQ ID NO: 5 - DNA, 22 bp, Artificial Sequence
  SoSUS1 R
tccagtggctcgaatctgtctg

SEQ ID NO: 6 - DNA, 19 bp, Artificial Sequence
  SoSUS2 F
gtgcggtttgccaacaatt

SEQ ID NO: 7 - DNA, 24 bp, Artificial Sequence
  SoSUS2 R
aaatatctgcagccttgtcactgt

SEQ ID NO: 8 - DNA, 24 bp, Artificial Sequence
  SoSUS4 F
cataacaggactggttgaagcttt

SEQ ID NO: 9 - DNA, 25 bp, Artificial Sequence
  SoSUS4 R
ccttggacttcttgacatcattgta

SEQ ID NO: 10 - DNA, 23 bp, Artificial Sequence
  SoSUS5 F
cacatattcattccattgagacc

SEQ ID NO: 11 - DNA, 23 bp, Artificial Sequence
  SoSUS5 R
tgtaaccatgtacactttcagtc

SEQ ID NO: 12 - DNA, 21 bp, Artificial Sequence
  SoSUS6 F
atgtactggaacagaatgtcc

SEQ ID NO: 13 - DNA, 21 bp, Artificial Sequence
  SoSUS6 R
tgaaggttgtagaacatttgt

SEQ ID NO: 14 - DNA, 17 bp, Artificial Sequence
  GAPDH F
cacggccactggaagca

SEQ ID NO: 15 - DNA, 20 bp, Artificial Sequence
  GAPDH R
tcctcagggttcctgatgcc

SEQ ID NO: 16 - DNA, 365 bp, Sorghum bicolor
gcggccgcagccctccagcaagtgaccccgcggcggctggagacctgatgagcgaaagggagcacttggagtcgtgttttttttccttcccctgatccggaggccaaaaaagagtctgcttttcttcctaggcggcgggcgttcgttgctgctcttcccttcaagcattgttattaccttgtcaaggtcttgttccatcattgatccgggtgttggttttagtagtctgatctgaattgttagtagtttgggttgagtcgagcggttgagagggatgttgggacttggcgcccttttccctgaaataagagtagcatccttgtggttcactttgcacctggaatcgatgtttgcctcagggtacc

SEQ ID NO: 17 - DNA, 374 bp, Sorghum bicolor
gcggccgctgtgggaaagaagaaccccaatctggagtagtggagaaccatcatctgcatttcgattgttcgctgcaattcgcattgttagttgtgtatttgagttatgtgtacttggtttccaagcactttggttcctttttgcgagttttgggcagcgctggctggttccttttataggaattagctgcaccttttgcttcaaataaacgcctgctcgttcacctgtcttccaaagttcaatgcaatgttttgttgcccaagtcttcatttctgactgatggtgatgttatgttctgtcagttctgttaatcacctgtttaatgtggtaggctgatgcctgttcttattatcaaaggttgctgtgccggtacc

SEQ ID NO: 18 - DNA, 263 bp, Sorghum bicolor
gcggccgctggtcgtcccctttggtgctcgtagcttgctcaactgttactgtgtaccacttggtacaaactgaaccttatcgcagggaaggaccttcagtaacttaggtgcggcagacggtagctaataaaatgtgcatatgcgctcgtttgtcttatgctgaactgaaccttgtgcctccctggctatattggttgaacatctaggtttattatgtacataaggcagtatgtgatccacctgtagcgtcaggctacggtacc

SEQ ID NO: 19 - DNA, 31 bp, Artificial Sequence
  Kpn Intro FW
ggggtaccacccgggtgatgcggtaactgat

SEQ ID NO: 20 - DNA, 30 bp, Artificial Sequence
  Bam Intro RV
gcgggatcctcccgggcttcaacctgcaga

SEQ ID NO: 21 - DNA, 29 bp, Artificial Sequence
  SUS 1 Pac I FW
ccttaattaaagccctccagcaagtgacc

SEQ ID NO: 22 - DNA, 29 bp, Artificial Sequence
  SUS 1 Bam HI RV
cgggatccctgaggcaaacatcgattcca

SEQ ID NO: 23 - DNA, 31 bp, Artificial Sequence
  SUS 2 Pac I FW
ccttaattaatgtgggaaagaagaaccccaa

SEQ ID NO: 24 - DNA, 33 bp, Artificial Sequence
  SUS 2 Bam HI RV
cgggatccggcacagcaacctttgataataaga

SEQ ID NO: 25 - DNA, 35 bp, Artificial Sequence
  SUS 3 Pac I FW
ccttaattaatggttcaatcgaaagtttgctttat

SEQ ID NO: 26 - DNA, 29 bp, Artificial Sequence
  SUS 3 Bam HI RV
cgggatccgtagcctgacgctacaggtgg

SEQ ID NO: 27 - DNA, 19 bp, Artificial Sequence
  SUS 1-4 F
tggtccggctgagatcatc

SEQ ID NO: 28 - DNA, 22 bp, Artificial Sequence
  SUS 1-4 R
tccagtggctcgaatctgtctg

SEQ ID NO: 29 - DNA, 19 bp, Artificial Sequence
  SUS 2-4 F
gtgcggtttgccaacaatt

SEQ ID NO: 30 - DNA, 24 bp, Artificial Sequence
  SUS 2-4 R
aaatatctgcagccttgtcactgt

SEQ ID NO: 31 - DNA, 24 bp, Artificial Sequence
  SUS 4-2 F
cataacaggactggttgaagcttt

SEQ ID NO: 32 - DNA, 25 bp, Artificial Sequence
  SUS 4-2 R
ccttggacttcttgacatcattgta

SEQ ID NO: 33 - DNA, 17 bp, Artificial Sequence
  GAPDH FW
cacggccactggaagca

SEQ ID NO: 34 - DNA, 20 bp, Artificial Sequence
  GAPDH RV
tcctcagggttcctgatgcc

SEQ ID NO: 35 - DNA, 17 bp, Sorghum bicolor
gtggtccggctgagatc

SEQ ID NO: 36 - DNA, 21 bp, Sorghum bicolor
cagacagattcgagccactgg

SEQ ID NO: 37 - DNA, 17 bp, Sorghum bicolor
gtggccctgctgaaata

SEQ ID NO: 38 - DNA, 21 bp, Sorghum bicolor
aaggcagacccaagctactgg

SEQ ID NO: 39 - DNA, 17 bp, Sorghum bicolor
gaggaccagctgagatt

SEQ ID NO: 40 - DNA, 21 bp, Sorghum bicolor
aagcaagacccaaataactgg

SEQ ID NO: 41 - DNA, 17 bp, Sorghum bicolor
gagggccagcagagatc

SEQ ID NO: 42 - DNA, 21 bp, Sorghum bicolor
aaggaagacccaagctattgg

SEQ ID NO: 43 - DNA, 17 bp, Sorghum bicolor
gaggccccgcagaaatc

SEQ ID NO: 44 - DNA, 21 bp, Sorghum bicolor
aacgaagatcccatgtactgg

SEQ ID NO: 45 - DNA, 19 bp, Sorghum bicolor
ctgtggcctgccgacgttc

SEQ ID NO: 46 - DNA, 22 bp, Sorghum bicolor
gggcgacaaggcgtcggccctg

SEQ ID NO: 47 - DNA, 19 bp, Sorghum bicolor
gtgcggtttgccaacaatt

SEQ ID NO: 48 - DNA, 22 bp, Sorghum bicolor
cagtgacaaggctgcagatatt

SEQ ID NO: 49 - DNA, 19 bp, Sorghum bicolor
ctgtggacttcctactttt

SEQ ID NO: 50 - DNA, 22 bp, Sorghum bicolor
ccccgagcaggctgctaatttg

SEQ ID NO: 51 - DNA, 19 bp, Sorghum bicolor
ctgcggattgacaaccttt

SEQ ID NO: 52 - DNA, 22 bp, Sorghum bicolor
tggcagggaggcaagcaacaag

SEQ ID NO: 53 - DNA, 19 bp, Sorghum bicolor
ctgtgggctgccaaccttt

SEQ ID NO: 54 - DNA, 22 bp, Sorghum bicolor
tggcaaagaggcaagcaacaag

SEQ ID NO: 55 - DNA, 24 bp, Sorghum bicolor
cttgactggtctggtggagctgta

SEQ ID NO: 56 - DNA, 22 bp, Sorghum bicolor
gaccacggcaacccttccaagg

SEQ ID NO: 57 - DNA, 24 bp, Sorghum bicolor
catgacaggcttggttgagatgta

SEQ ID NO: 58 - DNA, 22 bp, Sorghum bicolor
gaccatggcaaggaatcgaagg

SEQ ID NO: 59 - DNA, 24 bp, Sorghum bicolor
cataacaggactggttgaagcttt

SEQ ID NO: 60 - DNA, 25 bp, Sorghum bicolor
tacaatgatgtcaagaagtccaagg

SEQ ID NO: 61 - DNA, 24 bp, Sorghum bicolor
tatcactggactagtggagtggta

SEQ ID NO: 62 - DNA, 25 bp, Sorghum bicolor
ctgctggaagcatcgcagtccaagg

SEQ ID NO: 63 - DNA, 24 bp, Sorghum bicolor
catcactgggctggttgaatggta

SEQ ID NO: 64 - DNA, 25 bp, Sorghum bicolor
ctcctggaccccacgaaatccaagg

SEQ ID NO: 65 - DNA, 22 bp, Sorghum bicolor
gtggtgtgtgtgcagtcgggtg

SEQ ID NO: 66 - DNA, 22 bp, Sorghum bicolor
gagtagcatccttgtggttcac

SEQ ID NO: 67 - DNA, 23 bp, Sorghum bicolor
gtgatgttatgttctgtcagttc

SEQ ID NO: 68 - DNA, 22 bp, Sorghum bicolor
cgggtcaatgtggaagcccgag

SEQ ID NO: 69 - DNA, 22 bp, Sorghum bicolor
tcataaaaggctggctgtacaa

SEQ ID NO: 70 - DNA, 23 bp, Sorghum bicolor
cacatattcattccattgagacc

SEQ ID NO: 71 - DNA, 23 bp, Sorghum bicolor
gactgaaagtgtacatggttaca

SEQ ID NO: 72 - DNA, 21 bp, Sorghum bicolor
agccactggaacaagatctcc

SEQ ID NO: 73 - DNA, 21 bp, Sorghum bicolor
ggagatgctgtacgcgctcaa

SEQ ID NO: 74 - DNA, 21 bp, Sorghum bicolor
agctactgggacaagatctca

SEQ ID NO: 75 - DNA, 21 bp, Sorghum bicolor
tgagatgttctatgctctgaa

SEQ ID NO: 76 - DNA, 21 bp, Sorghum bicolor
aataactgggtgaaaatatct

SEQ ID NO: 77 - DNA, 20 bp, Sorghum bicolor
cgagatgttctacatatgaa

SEQ ID NO: 78 - DNA, 21 bp, Sorghum bicolor
agctattggaacaaggtgtcc

SEQ ID NO: 79 - DNA, 21 bp, Sorghum bicolor
gcagatgttctacaatcttca

SEQ ID NO: 80 - DNA, 21 bp, Sorghum bicolor
atgtactggaacagaatgtcc

SEQ ID NO: 81 - DNA, 22 bp, Sorghum bicolor
acaaatgttctacaaccttcat

(19) United States
(12) Patent Application Publication
(10) Pub. No.: US 2018/0355366 A1
(43) Pub. Date: Dec. 13, 2018

(54) YIELD PROMOTER TO INCREASE SUCROSE AND SUCROSE DERIVATIVES IN PLANTS

(71) Applicant: THE UNIVERSITY OF QUEENSLAND, St Lucia (AU)

(72) Inventor: Luguang WU, Taringa (AU)

(21) Appl. No.: 15/546,412

(22) PCT Filed: Jan. 29, 2015
(86) PCT No.: PCT/AU2015/050029

§ 371 Date: Dec. 18, 2017

(30) Foreign Application Priority Data
Jan. 29, 2014 (AU) 2014900262

(51) Int. Cl. C12N 15/82; A01H 1/04; A01H 4/00; A01H 1/06; C12N 15/10

(52) U.S. Cl. CPC C12N 15/8246; C12N 15/8261; C12N 15/8218; C12N 15/1024; A01H 4/008; A01H 1/06; A01H 1/04

(57) ABSTRACT

Disclosed are plants with improved carbohydrate content. More particularly, the present invention discloses sucrose-accumulating crop plants with increased content of sucrose and sucrose derivatives through inhibiting or abrogating expression of an endogenous member of a specific sucrose synthase gene subfamily.

### CROSS-REFERENCE TO RELATED APPLICATIONS

This application is the U.S. national phase of International Application No. PCT/AU2015/050029 filed Jan. 29, 2015 which designated the U.S., the entire contents of which are incorporated herein by reference.

### REFERENCE TO SEQUENCE LISTING SUBMITTED ELECTRONICALLY VIA EFS-WEB

The content of the electronically submitted sequence listing (Name: SEQ1.txt; Size: 28 kilobytes) filed on Dec. 18, 2017 is incorporated herein by reference in its entirety.

### FIELD OF THE INVENTION

This invention relates generally to plants with improved carbohydrate content. More particularly, the present invention relates to sucrose-accumulating crop plants with increased content of sucrose and sucrose derivatives through inhibiting or abrogating expression of an endogenous member of a specific sucrose synthase gene subfamily.

### BACKGROUND OF THE INVENTION

In higher plants, the organic carbon fixed by photosynthesis is transported primarily in the form of sucrose, from the photosynthetic source organs to sink organs where it may be stored directly, or converted into other storage compounds, or metabolized to provide ultimately all of the carbon and energy needed for life, or remobilized to other locations in the plant. Sucrose is a disaccharide involving a glycosidic bond between the reducing ends of glucose and fructose. This provides a high-energy molecule with high solubility and relatively limited chemical reactivity; ideal for its functions in plants and for a number of uses by humans.

Plants comprise the bulk of global biomass, in which almost all organic carbon has ultimately passed through sucrose. Another consequence of this central role in photosynthetic carbon capture and transport is that sucrose is the most abundant sugar on earth. Certain plants including sugarcane, sugar beet and sweet sorghums preferentially accumulate sucrose to high concentrations in storage organs. Industrially, sucrose is extracted from such plants for use as a human food; and as a feedstock for conversion into diverse organic molecules including sugars and sugar derivatives, polymers, and alcohols used as beverages, solvents, fuels and substrates for further manufacturing steps. Sucrose is also used industrially for in-planta conversion into other sugars and sugar derivatives with higher commercial value than sucrose.

Accordingly, there is a long history of human endeavor to increase the concentration and yield of sucrose in harvestable plant storage organs. For example, humans have since prehistoric times selected for sweeter variants of Saccharum species that are the parents of modern sugarcane cultivars. More recently there has been systematic work on the same goal through plant breeding and selection. Even more recently, there has been an effort to understand the biochemical, physiological and molecular genetic basis for plant sugar metabolism, and to apply this understanding through plant improvement using gene technologies.

The difficulty of this task is indicated by the historical pattern of sugar yields from most advanced sugarcane industries. Commercially, because of factors including transport and extraction costs, an increase in sugar concentration is highly advantageous over an equivalent increase in sugar yield per unit of farmed land area obtained through increased sugarcane plant yield. Nevertheless, over recent decades there has been a plateau in sugar concentration and limited gains in sugar yield have been obtained through increased plant yield. This has led to the hypothesis that a physiological ceiling has been reached for stored sucrose concentration. However, physiological considerations indicate that actual sugar concentrations and yields are well below theoretical limits.

Sugarcane has been used as an example because it is the predominant sugar crop, contributing about 75% of global industrial sugar production. However, the same concepts apply to ancillary sugar crops including beets and sweet sorghums that are cultivated in some environments unsuited to sugarcane.

The core biochemical reactions and corresponding enzymatic activities in the metabolism of sucrose within source and sink tissues are well understood, as summarized in FIG. 1 (Wind et al., 2010. Phytochemistry 71:1610-1614). The hexose sugars glucose (Glc) and fructose (Fru) and their energetically activated forms such as UDP-Glc are provided in source cells by photosynthetic carbon and energy capture. Sucrose is synthesized primarily from Fru and UDP-Glu, in a thermodynamically irreversible two-step reaction catalyzed by sucrose phosphate synthase (SPS) and sucrose phosphate phosphatase (SPP) in the cytosol. Sucrose can be cleaved by the enzyme known as sucrose synthase (SUS), to yield Fru and UDP-Glc. This reaction conserves the energy in the glycosidic bond and is thermodynamically reversible. Indeed SUS functions to synthesize sucrose when the ratio of Fru and UDP-Glc to sucrose is high; but in tissues with high sucrose concentration it is understood to act entirely by cleavage, to provide precursors for other diverse cellular processes including respiration and biosynthesis of cell walls and starch. SUS has long been considered as a cytosolic enzyme, but there is recent evidence for isoforms that can associate with the plasmalemma, the tonoplast and possibly other sub-cellular compartments (Vargas et al., 2010. Plant Sci. 178(1):1-8).

Sucrose can also be cleaved to yield Glc and Fru, by cellular invertase enzymes. This cleavage loses the energy in the glycosidic bond, and is therefore thermodynamically irreversible. There are multiple invertase enzymes in plant cells, classified originally based on their pH optimum. Structurally related acid invertases are localized in the cell wall (CWI) and the vacuole (VAI). Two broad families of invertases with alkaline or neutral pH optima (NI) are localized in the plastids or mitochondria (clade a), or in the cytosol, cell membranes or nucleus (clade (3) (Ji et al., 2005. J. Mol. Evol. 60(5):615-634; Vargas et al., 2010, supra). Both sucrose and hexoses can move between cells through plasmodesmata, and they can be carried across cellular membranes by specific sugar transporters. Given the central importance of sucrose in plants, it understandable that the enzymes and transporters involved in sucrose metabolism are highly regulated, though the details are not fully elucidated.

One driver for work to better understand plant sucrose metabolism has been the hope that it might enable the development of methods to enhance harvestable plant sucrose concentration and yield. An early approach was to engineer plants for increased or decreased expression of key activities within the process depicted in FIG. 1. Detailed modeling of enzyme kinetics and metabolite flux has been employed to indicate which of these multiple potential targets might exert the greatest control in the process, and therefore be preferred targets for manipulation. Comparisons of gene expression between plants with greater or less sucrose accumulation have been used at various levels of resolution from specific candidate activities in FIG. 1 to transcriptomic approaches facilitated by high-throughput sequencing. In sum, these studies have so far not yielded any method that has provided compelling evidence for enhanced sucrose concentration in the mature sugar-storage tissue of sugarcane, relative to existing elite cultivars as reference material, without severe adverse effects on plant growth or development that would overall substantially decrease the recoverable sugar yield. Transcriptomic approaches have revealed a vast array of differentially expressed genes, beyond current capabilities for experimental testing to discern any that might be useful in practice to achieve the practical goal. Modeling approaches have reinforced the limitations to our knowledge about key parameters essential for reliable predictions about effects at whole-cell and whole-plant levels. Endogenous gene manipulations have sometimes been revealing through neutral or negative effects, but increased sugar yield has been elusive, as for attempts to enhance other primary yield components by single gene manipulations in other highly selected crop plants.

One feature that is overlooked in the simple description of FIG. 1 is the existence in plants of complex families of genes at separate loci that encode various forms of the key metabolic enzymes. This complexity was practically impossible to resolve by earlier approaches at the level of enzyme activity; but it is revealed by analysis of genome and transcriptome sequence databases. For example, taking rice and Arabidopsis as examples of monocot and dicot plants with completely sequenced genomes, there are 6-9 non-allelic neutral invertases and 6-6 non-allelic SUS isoforms. The detailed functions of all these families are yet to be elucidated, but it is clear that they can be differentially expressed and targeted within any species, and that the complement and developmental functions of gene family members varies between species. In the light of this emerging understanding, it is apparent that early work to alter the levels of key enzymes—for example by down-regulation of expression using sequence regions conserved across plant species—was likely far too coarse to achieve the practical goal. While it is reasonable to speculate that more precise development modulation of particular family members or combinations might be effective it is not obvious which family members or combinations to use. The substantial gene complexity and functional diversity, and the paucity of functional analysis in relevant crop plants, makes empirical testing impractical even in a species for which an efficient transformation system and assay for the desired phenotype are available.

### SUMMARY OF THE INVENTION

The present invention stems in part from the determination that a shorter, experimentally testable, set of candidate genes for modulating the yield of sucrose and sucrose derivatives may be identified through analysis of developmental expression levels of individual gene family members in closely related genotypes with differences in sucrose accumulation in the range of current elite cultivars of sucrose-accumulating crop species. From this analysis, the present inventors identified five sucrose synthase gene subfamilies expressed in sucrose-accumulating crop species. However, it was discovered that only one of these endogenous subfamilies, the SUS2 gene subfamily, can be used effectively to increase the concentration or yield of sucrose or sucrose derivatives in harvestable plant storage organs through inhibiting expression of one more genes of that subfamily.

Accordingly, in one aspect, the present invention provides methods for increasing the concentration or yield of sucrose or sucrose derivatives in a plant, plant part or plant organ (e.g. plant stem) of a sucrose-accumulating crop plant. These methods generally comprise expressing in a cell (e.g., a plant stem cell) of the plant, plant part or plant organ a polynucleotide that comprises a nucleic acid sequence encoding an expression product that inhibits expression of a SUS2 nucleic acid molecule, or reduces the level or activity a SUS2 polypeptide, to thereby increase the concentration or yield of sucrose or sucrose derivatives in the plant, plant part or plant organ,

wherein the SUS2 nucleic acid molecule comprises, consists or consists essentially of a nucleotide sequence selected from the group consisting of:

(a) a nucleotide sequence that encodes the amino acid sequence:

[SEQ ID NO: 2]
MAAKLTRLHSLRERLGATFSSHPNELIALFSRYVNQGKGMLQRHQLLAEF
DALFDSDKEKYAPFEDFLRAAQEAIVLPPWVALAIRPRPGVWDYIRVNVS
ELAVEELSVSEYLAFKEQLVDGNSNSNFVLELDFEPFNASFPRPSMSKSI
GNGVQFLNRHLSSKLFQDKESLYPLLNFLKAHNYKGTTMMLNDRIQSLRG
LQSSLRKAEEYLLSVPQDTPYSEFNHRFQELGLEKGWGDTAKRVLDTLHL
LLDLLEAPDPANLEKFLGTIPMMFNVVILSPHGYFAQSNVLGYPDTGGQV
VYILDQVRALENEMLLRIKQQGLDITPKILIVTRLLPDAVGTTCGQRLEK
VIGTEHTDIIRIPFRNENGILRKWISRFDVWPYLETYTEDVASEIMLEMQ
AKPDLIVGNYSDGNLVATLLAHKLGVTQCTIAHALEKTKYPNSDIYLDKF
DSQYHFSCQFTADLIAMNHTDFIITSTFQEIAGSKDTVGQYESHIAFTLP
GLYRVVHGIDVFDPKFNIVSPGADMSVYYPYTETDKRLTAFHPEIEELIY
SDVENDEHKFVLKDKNKPIIFSMARLDRVKNMTGLVEMYGKNARLRELAN
LVIVAGDHGKESKDREEQAEFKKMYSLIDEYNLKGHIRWISAQMNRVRNA
ELYRYICDTKGAFVQPAFYEAFGLTVIESMTCGLPTIATCHGGPAEIIVD
GVSGLHIDPYHSDKAADILVNFFEKCKADPSYWDKISQGGLQRIYEKYTW
KLYSERLMTLTGVYGFWKYVSNLERRETRRYLEMFYALKYRSLASAVPLS
FD;

(b) a nucleotide sequence that encodes an amino acid sequence that corresponds to SEQ ID NO:2, for example, one that shares at least 90% (and at least 91% to at least 99% and all integer percentages in between) sequence similarity or sequence identity with the sequence set forth in SEQ ID NO:2;

(c) a nucleotide sequence selected from:

[SEQ ID NO:1]
ttgcccgtcagtgagtcgtattacaccgggtggatggcccggccgacgcg
tccgatctgtcccagttctctgttctgttctgtcgacgccattcctgtgc
tctgccgtcccagcgtttgccaagtattgagtgtcattgagccatggctg
ccaagttgactcgcctccacagtcttcgcgaacgccttggtgccaccttc
tcctctcatcccaatgagctgattgcactcttctccaggtatgttaacca
gggcaagggaatgcttcagcgccatcaactgcttgctgagtttgatgccc
tgtttgatagtgacaaggagaagtatgcgcccttcgaagactttcttcgt
gctgctcaggaagcaattgtgctccctccctgggtagcacttgctatcag
gccaaggcctggtgtctgggattacattcgagtgaatgtaagcgagttgg
ctgtggaggagctgagtgtttctgagtacttggcattcaaggaacagctg
gtggatggaaattccaacagcaactttgttcttgagcttgattttgagcc
cttcaatgcctcattccctcgtccttccatgtcaaagtccattggaaatg
gagtgcaattccttaaccgacacctgtcttccaagttgttccaggacaag
gagagcctgtacccattgctgaatttcctcaaagcccataactacaaggg
cacgacgatgatgttgaatgacagaattcagagcctccgtgggctccagt
catcccttagaaaggcagaagagtatctactgagtgtccctcaagacact
ccctactcagagttcaaccataggttccaagagcttggcttggagaaggg
ttggggtgacactgcaaagcgcgtacttgatacactccacttgcttcttg
accttcttgaggcccctgatcctgccaacttggagaagttccttggaact
ataccaatgatgttcaatgttgttatcctgtctcctcatggctactttgc
ccaatccaatgtgcttggataccctgacactggtggtcaggttgtgtaca
ttttggatcaagtccgtgctttggagaatgagatgcttcttaggattaag
cagcaaggccttgacatcaccccgaagatcctcattgttaccaggctgtt
gcctgatgctgttgggactacgtgcggtcagcgtctggagaaggtcattg
gaaccgagcacacagacattattcgtattccattcagaaatgagaatggt
attctccgcaagtggatctctcgttttgatgtctggccatacctggagac
atacactgaggatgttgccagtgaaataatgttagaaatgcaggccaagc
ctgaccttattgttggcaactacagtgatggcaatctagtcgccactctg
ctcgcgcacaagttgggagttactcagtgtaccattgcccacgccttgga
gaaaaccaaatatcccaactcagacatatacttagacaaatttgacagcc
aataccacttctcatgccagttcacagctgaccttattgccatgaatcac
actgatttcatcatcaccagtacattccaagaaatcgcgggaagcaagga
cactgtggggcagtatgagtcccacattgcgttcactcttcctggacttt
accgtgttgtccatggcattgatgtttttgatcccaaattcaacattgtc
tctcctggagcagacatgagtgtttactacccatacactgaaactgacaa
gagactcactgccttccatcctgaaattgaggagctcatctacagtgatg
ttgagaatgatgagcacaagtttgtgttgaaggacaagaacaagccgatc
atcttctcaatggctcgtcttgaccgtgtgaagaacatgacaggcttggt
tgagatgtatggtaagaatgcacgcctgagggaattggcaaaccttgtga
ttgttgctggtgaccatggcaaggaatcgaaggacagggaggagcaggca
gagttcaagaagatgtacagtctcattgatgagtacaacttgaagggcca
tatccggtggatctcagctcagatgaaccgtgtccgcaacgctgagttgt
accgctacatttgtgacacgaagggagcatttgtgcagcctgcattctat
gaagcattcggcctgactgtcattgagtccatgacgtgcggtttgccaac
aattgcaacctgccatggtggccctgctgaaataattgtggacggggtgt
ctggtttgcacattgatccttaccacagtgacaaggctgcagatattttg
gtcaacttctttgagaagtgcaaggcagacccaagctactgggacaagat
ctcacagggtggactgcagagaatttatgagaagtacacctggaagctct
actccgagaggctgatgaccctgactggtgtatacggattctggaagtat
gtgagcaatctggagaggcgtgagactcgccgctaccttgagatgttcta
tgctctgaaataccgtagcctggcaagtgcggttccattgtccttcgatt
agtgtgggaaagaagaaccccaatctggagtagtggagaaccatcatctg
catttcgattgttcgctgcaattcgcattgttagttgtgtatttgagtta
tgtgtacttggtttccaagcactttggttcctttttgcgagttttgggca
gcgctggctggttccttttataggaattagctgcaccttttgcttcaaat
aaacgcctgctcgttcacctgtcttccaaagttcaatgcaatgttttgtt
gcccaagtcttcatttctgactgatggtgatgttatgttctgtcagttct
gttaatcacctgtttaatgtggtaggctgatgcctgttcttattatcaaa
ggttgctgtgcc,
and
[SEQ ID NO: 3]
atggctgccaagttgactcgcctccacagtcttcgcgaacgccttggtgc
caccttctcctctcatcccaatgagctgattgcactcttctccaggtatg
ttaaccagggcaagggaatgcttcagcgccatcaactgcttgctgagttt
gatgccctgtttgatagtgacaaggagaagtatgcgcccttcgaagactt
tcttcgtgctgctcaggaagcaattgtgctccctccctgggtagcacttg
ctatcaggccaaggcctggtgtctgggattacattcgagtgaatgtaagc
gagttggctgtggaggagctgagtgtttctgagtacttggcattcaagga
acagctggtggatggaaattccaacagcaactttgttcttgagcttgatt
ttgagcccttcaatgcctcattccctcgtccttccatgtcaaagtccatt
ggaaatggagtgcaattccttaaccgacacctgtcttccaagttgttcca
ggacaaggagagcctgtacccattgctgaatttcctcaaagcccataact
acaagggcacgacgatgatgttgaatgacagaattcagagcctccgtggg
ctccagtcatcccttagaaaggcagaagagtatctactgagtgtccctca
agacactccctactcagagttcaaccataggttccaagagcttggcttgg
agaagggttggggtgacactgcaaagcgcgtacttgatacactccacttg
cttcttgaccttcttgaggcccctgatcctgccaacttggagaagttcct
tggaactataccaatgatgttcaatgttgttatcctgtctcctcatggct
actttgcccaatccaatgtgcttggataccctgacactggtggtcaggtt
gtgtacattttggatcaagtccgtgctttggagaatgagatgcttcttag
gattaagcagcaaggccttgacatcaccccgaagatcctcattgttacca
ggctgttgcctgatgctgttgggactacgtgcggtcagcgtctggagaag
gtcattggaaccgagcacacagacattattcgtattccattcagaaatga
gaatggtattctccgcaagtggatctctcgttttgatgtctggccatacc
tggagacatacactgaggatgttgccagtgaaataatgttagaaatgcag
gccaagcctgaccttattgttggcaactacagtgatggcaatctagtcgc
cactctgctcgcgcacaagttgggagttactcagtgtaccattgcccacg
ccttggagaaaaccaaatatcccaactcagacatatacttagacaaattt
gacagccaataccacttctcatgccagttcacagctgaccttattgccat
gaatcacactgatttcatcatcaccagtacattccaagaaatcgcgggaa
gcaaggacactgtggggcagtatgagtcccacattgcgttcactcttcct
ggactttaccgtgttgtccatggcattgatgtttttgatcccaaattcaa
cattgtctctcctggagcagacatgagtgtttactacccatacactgaaa
ctgacaagagactcactgccttccatcctgaaattgaggagctcatctac
agtgatgttgagaatgatgagcacaagtttgtgttgaaggacaagaacaa
gccgatcatcttctcaatggctcgtcttgaccgtgtgaagaacatgacag
gcttggttgagatgtatggtaagaatgcacgcctgagggaattggcaaac
cttgtgattgttgctggtgaccatggcaaggaatcgaaggacagggagga
gcaggcagagttcaagaagatgtacagtctcattgatgagtacaacttga
agggccatatccggtggatctcagctcagatgaaccgtgtccgcaacgct
gagttgtaccgctacatttgtgacacgaagggagcatttgtgcagcctgc
attctatgaagcattcggcctgactgtcattgagtccatgacgtgcggtt
tgccaacaattgcaacctgccatggtggccctgctgaaataattgtggac
ggggtgtctggtttgcacattgatccttaccacagtgacaaggctgcaga
tattttggtcaacttctttgagaagtgcaaggcagacccaagctactggg
acaagatctcacagggtggactgcagagaatttatgagaagtacacctgg
aagctctactccgagaggctgatgaccctgactggtgtatacggattctg
gaagtatgtgagcaatctggagaggcgtgagactcgccgctaccttgaga
tgttctatgctctgaaataccgtagcctggcaagtgcggttccattgtcc
ttcgattag;

(d) a nucleotide sequence that corresponds to SEQ ID NO:1 or 3, or a complement thereof, for example, one that shares at least 90% (and at least 91% to at least 99% and all integer percentages in between) sequence identity with the sequence set forth in SEQ ID NO:1 or 3, or a complement thereof; or

(e) a nucleotide sequence that hybridizes under at least medium stringency conditions to the sequence set forth in SEQ ID NO:1 or 3, or a complement thereof,

wherein the nucleotide sequence of (a), (b), (c), (d) or (e) encodes an amino acid sequence having sucrose synthase activity,

wherein the SUS2 polypeptides comprises, consists or consists essentially of an amino acid sequence selected from:

(i) the amino acid sequence set forth in SEQ ID NO:2;

(ii) an amino acid sequence that corresponds to SEQ ID NO:2, for example, one that shares at least 90% (and at least 91% to at least 99% and all integer percentages in between) sequence similarity or sequence identity with the sequence set forth in SEQ ID NO:2;

(iii) an amino acid sequence which is encoded by the nucleotide sequence set forth in any one of SEQ ID NO:1 or 3;

(iv) an amino acid sequence which is encoded by a nucleotide sequence that corresponds to SEQ ID NO: 1 or 3, or a complement thereof, for example, one that shares at least 90% (and at least 91% to at least 99% and all integer percentages in between) sequence identity with the sequence set forth in SEQ ID NO:1, or a complement thereof; or

(v) an amino acid sequence which is encoded by a nucleotide sequence that hybridizes under at least medium stringency conditions to the sequence set forth in SEQ ID NO:1 or 3, or a complement thereof,

wherein the amino acid sequence of (i), (ii), (iii), (iv) or (v) has sucrose synthase activity.

In some embodiments of the above aspects, the concentration or yield of sucrose or sucrose derivatives in the plant, plant part or plant organ is increased by at least about 5% (e.g., at least about 6%, 7%, 8%, 9%, 10%, 15% 20%, 25%, 30%, 40%, 50%, 60%, 70%, 80%, 90%) relative to the concentration or yield of sucrose or sucrose derivatives in a control plant, plant part or plant organ that does express the polynucleotide.

In a related aspect, the present invention provides methods for increasing the concentration or yield of sucrose or sucrose derivatives in a plant, plant part or plant organ (e.g. plant stem) of a sucrose-accumulating crop plant. These methods generally comprise introducing a nucleic acid construct into the genome of the plant to produce a transformed plant and regenerating therefrom a stably transformed plant, wherein the nucleic acid construct comprises in operable connection: (1) a promoter that is operable in a cell of the sucrose-accumulating crop plant (e.g., a plant stem cell); and (2) a nucleic acid sequence encoding an expression product that inhibits expression of a SUS2 nucleic acid molecule as broadly described above and elsewhere herein, or reduces the level or activity a SUS2 polypeptide as broadly described above and elsewhere herein. In some embodiments, the promoter is a stem-specific or stem-preferential promoter. In some embodiments, the expression product is a SUS2-inhibiting RNA molecule (e.g., siRNA, shRNA, microRNAs, antisense RNA etc.) that inhibits expression of a SUS2 nucleic acid molecule as broadly described above and elsewhere herein. In other embodiments, the expression product is an antibody (also referred to herein as a “SUS2 antibody”) that is immuno-interactive with a SUS2 polypeptide as broadly described above and elsewhere herein.

In some embodiments, these methods further comprise selecting a transformed plant that has an increased concentration or yield of sucrose or sucrose derivatives, as compared to a control plant that does not contain the nucleic acid construct. In some embodiments, the nucleic acid construct is introduced into regenerable plant cells so as to yield transformed plant cells, which are suitably identified and selected, and which are subsequently used for regenerating differentiated plants. Typically, a transformed plant cell line is selected from the transformed plant cells for the differentiation of a transgenic plant. In some embodiments, the regenerable cells are regenerable dicotyledonous plant cells. In other embodiments, the regenerable cells are regenerable monocotyledonous plant cells such as regenerable graminaceous monocotyledonous plant cells. In one example, the regenerable plant cells are regenerable sugarcane plant cells. Desirably, the nucleic acid construct is transmitted through a complete cycle of the differentiated transgenic plant to its progeny so that it is expressed by the progeny plants. Thus, the invention also provides seed, plant parts, tissue, and progeny plants derived from the differentiated transgenic plant.

In related aspects, the present invention provides SUS2-inhibiting RNA molecules as broadly defined above and elsewhere herein as well as SUS2 antibodies as broadly defined above and elsewhere herein for use in increasing the concentration or yield of sucrose or sucrose derivatives in a plant, plant part or plant organ (e.g. plant stem) of a sucrose-accumulating crop plant.

In other related aspects, the present invention provides methods for making a genetically modified plant having a decreased level of SUS2 compared to that of a control plant, wherein the genetically modified plant displays an increased concentration or yield of sucrose or sucrose derivatives in plant storage organs relative to the control plant. These methods generally comprise: providing at least one plant cell containing a SUS2 gene encoding a functional SUS2 polypeptide (e.g., a broadly described above and elsewhere herein); treating the at least one plant cell under conditions effective to inactivate the SUS2 gene, thereby yielding at least one genetically modified plant cell containing an inactivated SUS2 gene; and propagating the at least one genetically modified plant cell into a genetically modified plant, wherein the genetically modified plant has a decreased level of SUS2 polypeptide compared to that of the control plant and displays an increased concentration or yield of sucrose or sucrose derivatives in plant storage organs relative to the control plant. In some embodiments, the genetically modified plant is a sucrose-accumulating crop plant.

In another aspect, the present invention provides genetically modified sucrose-accumulating crop plants, plant parts or plant organs (e.g., plant stems) comprising plant cells (e.g., plant stem cells) comprising an inactivation of a SUS2 gene and displaying an increased concentration or yield of sucrose or sucrose derivatives relative to a control plant, plant part or plant organ. In some embodiments, the genetically modified plants, plant parts or plant organs are sucrose-accumulating crop plants, plant parts or plant organs.

In related aspects, the present invention provides genetically modified sucrose-accumulating crop plants, plant parts or plant organs (e.g., plant stem cells) comprising plant cells (e.g., plant stem cells) having a decreased level of SUS2 compared to that of a control plant, wherein the genetically modified plants, plant parts or plant organs have an increased concentration or yield of sucrose or sucrose derivatives relative to a control plant.

In other related aspects, the present invention provides isolated sucrose-accumulating crop plant cells containing a nucleic acid construct as broadly described above and elsewhere herein. In some embodiments, the plant cells have the nucleic acid construct incorporated into the plant genome.

In still other related aspects, the present invention provides transgenic sucrose-accumulating crop plants, plant parts or plant organs (e.g., plant stems) comprising plant cells (e.g., plant stem cells) as broadly described above and elsewhere herein, wherein the transgenic plants, plant parts or plant organs (e.g., plant stems) have an increased concentration or yield of sucrose or sucrose derivatives.

In yet another aspect, the invention contemplates plant breeding methods to transfer genetic material of a transgenic or genetically modified plant as broadly described above and elsewhere herein via crossing and backcrossing to other sucrose-accumulating crop plants. Typically, these methods will comprise the steps of: (1) crossing a plant containing that genetic material with a sucrose-accumulating crop plant; (2) recovering reproductive material from the progeny of the cross; and (3) growing plants with increased concentration or yield of sucrose or sucrose derivatives relative to control plants from the reproductive material. In some embodiments, the methods further comprise selecting for expression of a nucleic acid sequence corresponding to the nucleic acid construct as broadly described above and elsewhere herein (or an associated marker gene) among the progeny of the backcross. In other embodiments, the methods further comprise selecting for inactivation of a SUS2 gene among the progeny of the backcross.

In some embodiments of any of the aspects described above and elsewhere herein, the sucrose-accumulating crop plant is selected from sugar beet, corn, sugarcane and sorghum. In illustrative examples of this type, the sucrose-accumulating crop plant is a C4 plant (e.g., corn, sugarcane, sorghum, etc.).

### BRIEF DESCRIPTION OF THE DRAWINGS

FIG. 1 is schematic representation from Wind et al. (2010. Phytochemistry 71:1610-1614) showing an overview of the life and death of a sucrose molecule. Following synthesis in the source, sucrose is transported to the sink, where is can be utilized or stored. Sucrose transport depends on sucrose transporters, as indicated by black circles with arrows. The light grey transporter sign represents hexose transporters. Abbreviations are: Suc, sucrose; Fru, fructose; Glc, glucose; UDP-Glc, UDP-glucose; SPP, sucrose-phosphatase; SPS, sucrose-phosphate synthase; SUSY, sucrose synthase; CWINV, cell wall invertase; VINV, vacuolar invertase; CINV, cytosolic/plastidic/mitochondrial invertases.

FIG. 2 is a graphical representation showing a phylogenetic dendrogram comparison of deduced amino acid sequences of plant sucrose synthases (SUS). The phylogenetic dendrogram was generated using UPGMA based on identity. The deduced amino acid sequences of plants were obtained from NCBI and Phytozome.

FIG. 3 is a graphical representation showing the transcript levels of SUS genes in various sugarcane tissues. The sugarcane plant Q117 was 6-month old, comprising ratoons with 22 internodes grown under glasshouse conditions. L, leaf blades; in, Internodes; The numbers tailed with L and In are numbers from TVD. R, white young roots.

FIG. 4 is a graphical representation showing relative expression of SoSUS1 (a), SoSUS2 (b), SoSUS4 (c) or SoSUS5 (d), in stem and leaf tissues of the 4 high-CCS (the left 4 bars in each group) and 4 low-CCS (the right 4 bars in each group) lines. The samples were from 9 month old ratoons grown in the field. Values in each large panel are means of 3 reps±SE. Note the significant difference in comparisons in the right panels showing a nonparametric t test on average values of the internode 15 (a) and of the internode 7 (b) in the corresponding high- or low-CCS lines. Young leaf: non-photosynthesis sink leaf; Mature leaf: #3.

FIG. 5 is a graphical representation showing the relationships between sucrose contents in whole cane juice and SoSUS1 mRNA pool sizes (a, b, c) or SoSUS1 mRNA pool sizes (d, e, f) in internode 3 (a, d), internode 7 (b, e) and internode 15 (c, f) of the 4 high-CCS and 4 low-CCS lines shown in Table 7.

FIG. 6 is a graphical representation showing correlation between internode 15 SoSUS1 mRNA amounts and internode 7 SoSUS2 mRNA levels of the 4 high-CCS and 4 low-CCS lines shown in Table 6.

FIG. 7 is a graphical representation showing the relationships between sucrose contents in whole cane juice and SUS activities (breakage) in internode 3 (a), internode 7 (b) and internode 15 (c) of the 4 high-CCS and 4 low-CCS cultivars shown in Table 6.

FIG. 8 is a graphical representation showing the relationships between SUS activities (breakage) and SoSUS1 mRNA pool sizes (a, b, c) or SoSUS2 mRNA pool sizes (d, e, f) in internode 3 (a, d), internode 7 (b, e) and internode 15 (c, f) of the 4 high-CCS and 4 low-CCS lines shown in shown in Table 6.

FIG. 9 is a diagrammatic representation showing primer specificity of 5 sucrose synthase subfamilies. The order of DNA molecules in each panel represent the longest tentative consensus (TC) of each subfamily 1, 2, 4, 5, 6. The arrow point to the last base pair at the primer 3′ end; the primers from right panels are complementary.

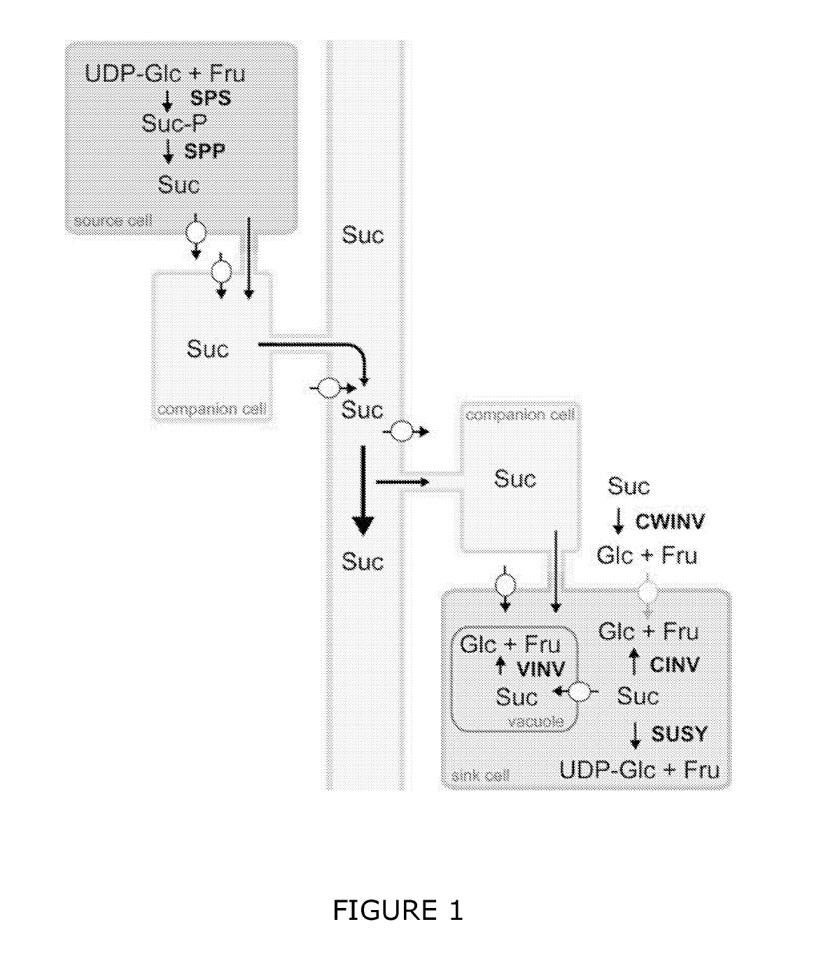

The nucleotide sequences shown are
(SEQ ID NO: 35)
GTGGTCCGGCTGAGATC,
(SEQ ID NO: 36)
CAGACAGATTCGAGCCACTGG,
(SEQ ID NO: 37)
GTGGCCCTGCTGAAATA,
(SEQ ID NO: 38)
AAGGCAGACCCAAGCTACTGG,
(SEQ ID NO: 39)
GAGGACCAGCTGAGATT,
(SEQ ID NO: 40)
AAGCAAGACCCAAATAACTGG,
(SEQ ID NO: 41)
GAGGGCCAGCAGAGATC,
(SEQ ID NO: 42)
AAGGAAGACCCAAGCTATTGG,
(SEQ ID NO: 43)
GAGGCCCCGCAGAAATC,
(SEQ ID NO: 44)
AACGAAGATCCCATGTACTGG,
(SEQ ID NO: 45)
CTGTGGCCTGCCGACGTTC,
(SEQ ID NO: 46)
GGGCGACAAGGCGTCGGCCCTG,
(SEQ ID NO: 47)
GTGCGGTTTGCCAACAATT,
(SEQ ID NO: 48)
CAGTGACAAGGCTGCAGATATT,
(SEQ ID NO: 49)
CTGTGGACTTCCTACTTTT,
(SEQ ID NO: 50)
CCCCGAGCAGGCTGCTAATTTG,
(SEQ ID NO: 51)
CTGCGGATTGACAACCTTT,
(SEQ ID NO: 52)
TGGCAGGGAGGCAAGCAACAAG,
(SEQ ID NO: 53)
CTGTGGGCTGCCAACCTTT,
(SEQ ID NO: 54)
TGGCAAAGAGGCAAGCAACAAG,
(SEQ ID NO: 55)
CTTGACTGGTCTGGTGGAGCTGTA,
(SEQ ID NO: 56)
GACCACGGCAACCCTTCCAAGG,
(SEQ ID NO: 57)
CATGACAGGCTTGGTTGAGATGTA,
(SEQ ID NO: 58)
GACCATGGCAAGGAATCGAAGG,
(SEQ ID NO: 59)
CATAACAGGACTGGTTGAAGCTTT,
(SEQ ID NO: 60)
TACAATGATGTCAAGAAGTCCAAGG,
(SEQ ID NO: 61)
TATCACTGGACTAGTGGAGTGGTA,
(SEQ ID NO: 62)
CTGCTGGAAGCATCGCAGTCCAAGG,
(SEQ ID NO: 63)
CATCACTGGGCTGGTTGAATGGTA,
(SEQ ID NO: 64)
CTCCTGGACCCCACGAAATCCAAGG,
(SEQ ID NO: 65)
GTGGTGTGTGTGCAGTCGGGTG,
(SEQ ID NO: 66)
GAGTAGCATCCTTGTGGTTCAC,
(SEQ ID NO: 67)
GTGATGTTATGTTCTGTCAGTTC,
(SEQ ID NO: 68)
CGGGTCAATGTGGAAGCCCGAG,
(SEQ ID NO: 69)
TCATAAAAGGCTGGCTGTACAA,
(SEQ ID NO: 70)
CACATATTCATTCCATTGAGACC,
(SEQ ID NO: 71)
GACTGAAAGTGTACATGGTTACA,
(SEQ ID NO: 72)
AGCCACTGGAACAAGATCTCC,
(SEQ ID NO: 73)
GGAGATGCTGTACGCGCTCAA,
(SEQ ID NO: 74)
AGCTACTGGGACAAGATCTCA,
(SEQ ID NO: 75)
TGAGATGTTCTATGCTCTGAA,
(SEQ ID NO: 76)
AATAACTGGGTGAAAATATCT,
(SEQ ID NO: 77)
CGAGATGTTCTACATATGAA,
(SEQ ID NO: 78)
AGCTATTGGAACAAGGTGTCC,
(SEQ ID NO: 79)
GCAGATGTTCTACAATCTTCA,
(SEQ ID NO: 80)
ATGTACTGGAACAGAATGTCC,
and
(SEQ ID NO: 81)
ACAAATGTTCTACAACCTTCAT.

FIG. 10 is a graphical representation showing Brix values in mature stem tissues (a-c) and stock fresh weights (d-f) of transgenic sugarcane lines with different down-regulating construct compared to control Q117. Plants grew in 2 L soil small pots under glasshouse condition for 11 months. Bars represent means (n=10)±SEM. S2: SUS 2 hairpin construct; S2N1: co-transformation of SUS 2 hairpin and N1 hairpin construct; S2N2: co-transformation of SUS 2 hairpin and N2 hairpin construct; S3N1: co-transformation of SUS 3 hairpin and N1 hairpin construct; S3N2: co-transformation of SUS 3 hairpin and N2 hairpin construct. Significant differences by ANOVA with Bonferroni post-tests are marked: * for P<0.05, ** for P<0.01 or *** for P<0.001.

FIG. 11 is a graphical representation showing Brix values of internode 16 (a, b), and stem fresh weight (c, d) in the SUS2 down-regulating transgenic lines and Q117 controls of the second generation. The plants were grown in 2 L soil pots on block 1 (a, c) and on block 2 (b, d) in constraint glasshouse conditions for 12 months. Results are means of three replicated plants with standard error bars. Significant differences by ANOVA with Bonferroni post-tests are marked: * for P<0.05, ** for P<0.01 or *** for P<0.001.

FIG. 12 is a graphical representation showing Brix values of internode 16 (a), stem fresh weight (b) and internode numbers (c) in the main stalks of the transgenic lines and Q117controls of the second generation. The plants grew in constraint glasshouse conditions for 5 months in 2 L soil pots and moved to 333 L soil large posts for 6 months. Results, are means of three replicated plants with standard error bars. Significant differences by ANOVA with Bonferroni post-tests are marked: * for P<0.05, ** for P<0.01 or *** for P<0.001.

FIG. 13 is a graphical representation showing sucrose contents (a, b) in different developmental stages and stalk fresh weight (c) as well as internode numbers (d) in the secondary stalks of the transgenic lines and Q117 controls. The plants grew in constraint glasshouse conditions in the 333 L soil large pots for 12-13 months. Solid lines in panels a and b represent first wave of sampling when the plants were 12 months old. Dot lines in panel b represent second sampling when plants were 13 months old. FQ117 stands for the planting setts were from field, while all other planting materials were from glasshouse grown setts. Results are means of three replicated plants with standard error bars.

FIG. 14 is a photographic and graphical representation showing a Northern blot of internode 13 of transgenic line A and control Q117. Total RNA was individually extracted from 2 plants of transgenic line A and the control. The plants grew in constraint glasshouse conditions in 333 L soil pots for 12 months. Top panel: twenty mg RNA each lane was run on a 0.8% agarose gel blotted with 1 kb SUS2 probe that has conserved regions for both SUS2 and SUS1. Middle panel: Ethidium bromide staining after electrophoresis to show the loading amount of each lane. Bottom panel: quantification of the top panel.

FIG. 15 is a graphical representation showing correlations between Brix values and relative expression of SUS1 (a), SUS2 (b), and SUS4 (c). The total RNAs were extracted from internode 15 of each SUS down-regulating or control Q117 plants of the first generation grown in 2 L soil small pots under glasshouse conditions at 12 months old. GAPDH was used as internal control for each internodes on the transgenic plants and the controls.

FIG. 16 is a graphical representation showing expression levels of SUS1 (a, d), SUS2 (b, e) and SUS4 (c, f) in different developmental stages of the transgenic line A and control Q117. GAPDH was used as internal control for each internodes on the transgenic plants and the controls. The expression levels were presented as Delta Ct (a-c, Delta Ct=Ct (SUS)−Ct(GAPDH)) and reduced folds (d-f, Folds=2^{[Ct(Q117, SUS)-Ct(Q117, GAPDH)]/2[Ct(A, SUS)-Ct(A, GAPDH)])}. The plants grew in constraint glasshouse conditions in the 333 L soil large pots for 12 months. Results, expressed per mg protein, are means of three replicated plants with standard error bars. Significant differences by ANOVA with Bonferroni post-tests are marked: * for P<0.05, ** for P<0.01 or *** for P<0.001.

FIG. 17 is a graphical representation showing activities of SUS enzymes in breakage (a) and synthesis (b) directions in different developmental stages of the transgenic line A and control Q117. The plants grew in constraint glasshouse conditions in the 333 L soil large pots for 12 months. Results, expressed per mg protein, are means of three replicated plants with standard error bars. Significant differences by ANOVA with Bonferroni post-tests are marked: * for P<0.05, ** for P<0.01 or *** for P<0.001.

FIG. 18 is a schematic representation showing a hairpin structure comprising SUS sense and antisense fragments and intervening intron.

FIG. 19 is a schematic representation showing a map of a construct comprising one embodiment of a hairpin structure operably connected to the ShortA1 (1.2 Kb) promoter.

FIG. 20 is a graphical representation showing Brix values of internode 16 (a), stem fresh weight (b) and internode numbers (c) in the main stalks of the transgenic lines and Q117controls of the ratoon (third vegetitive generation from the second generation of planting). The plants grew in constrained glasshouse conditions for 11 months in 2 L soil pots. Results, are means of four replicated plants with standard error bars. Significant differences by ANOVA with Bonferroni post-tests are marked: * for P<0.05, ** for P<0.01 or *** for P<0.001.

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element. Thus, for example, the term “construct sequence” also includes a plurality of constructs.

As used herein, “and/or” refers to and encompasses any and all possible combinations of one or more of the associated listed items, as well as the lack of combinations when interpreted in the alternative (or).

Further, the term “about,” as used herein when referring to a measurable value such as an amount of a compound or agent, dose, time, temperature, activity, level, number, frequency, percentage, dimension, size, amount, weight, position, length and the like, is meant to encompass variations of ±20%, ±10%, ±5%, ±1%, ±0.5%, or even ±0.1% of the specified amount.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like.

The term “antisense” refers to a nucleotide sequence whose sequence of nucleotide residues is in reverse 5′ to 3′ orientation in relation to the sequence of deoxynucleotide residues in a sense strand of a DNA duplex. A “sense strand” of a DNA duplex refers to a strand in a DNA duplex which is transcribed by a cell in its natural state into a “sense mRNA.” Thus an “antisense” sequence is a sequence having the same sequence as the non-coding strand in a DNA duplex. The term “antisense RNA” refers to a RNA transcript that is complementary to all or part of a target primary transcript or mRNA and that blocks the expression of a target gene by interfering with the processing, transport and/or translation of its primary transcript or mRNA. The complementarity of an antisense RNA may be with any part of the specific gene transcript, in other words, at the 5′ non-coding sequence, 3′ non-coding sequence, introns, or the coding sequence. In addition, as used herein, antisense RNA may contain regions of ribozyme sequences that increase the efficacy of antisense RNA to block gene expression. “Ribozyme” refers to a catalytic RNA and includes sequence-specific endoribonucleases. “Antisense inhibition” refers to the production of antisense RNA transcripts capable of preventing the expression of the target protein.

The terms “cis-acting element,” “cis-acting sequence” or “cis-regulatory region” are used interchangeably herein to mean any sequence of nucleotides which modulates transcriptional activity of an operably linked promoter and/or expression of an operably linked nucleotide sequence. Those skilled in the art will be aware that a cis-sequence may be capable of activating, silencing, enhancing, repressing or otherwise altering the level of expression and/or cell-type-specificity and/or developmental specificity of any nucleotide sequence, including coding and non-coding sequences.

By “coding sequence” is meant any nucleic acid sequence that contributes to the code for the polypeptide product of a gene. By contrast, the term “non-coding sequence” refers to any nucleic acid sequence that does not contribute to the code for the polypeptide product of a gene.

As used herein, “complementary” polynucleotides are those that are capable of hybridizing via base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. For example, the sequence “A-G-T” binds to the complementary sequence “T-C-A.” It is understood that two polynucleotides may hybridize to each other even if they are not completely or fully complementary to each other, provided that each has at least one region that is substantially complementary to the other. The terms “complementary” or “complementarity,” as used herein, refer to the natural binding of polynucleotides under permissive salt and temperature conditions by base-pairing. Complementarity between two single-stranded molecules may be “partial,” in which only some of the nucleotides bind, or it may be complete when total complementarity exists between the single stranded molecules either along the full length of the molecules or along a portion or region of the single stranded molecules. The degree of complementarity between nucleic acid strands has significant effects on the efficiency and strength of hybridization between nucleic acid strands. As used herein, the terms “substantially complementary” or “partially complementary” mean that two nucleic acid sequences are complementary at least at about 50%, 60%, 70%, 80% or 90% of their nucleotides. In some embodiments, the two nucleic acid sequences can be complementary at least at about 85%, 90%, 95%, 96%, 97%, 98%, 99% or more of their nucleotides. The terms “substantially complementary” and “partially complementary” can also mean that two nucleic acid sequences can hybridize under high stringency conditions and such conditions are well known in the art.

Throughout this specification, unless the context requires otherwise, the words “comprise,” “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements. Thus, the term “consisting essentially of” when used in a claim of this invention is not intended to be interpreted to be equivalent to “comprising.” Thus, the term “consisting essentially of” (and grammatical variants), as applied to a nucleic acid sequence of this invention, means a polynucleotide that consists the recited sequence (e.g., SEQ ID NO) and a total of fifty or less (e.g., 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1) additional nucleotides on the 5′ and/or 3′ ends of the recited sequence such that the function of the polynucleotide is not materially altered. The total of fifty or less additional nucleotides includes the total number of additional nucleotides on both ends added together. The term “consisting essentially of” (and grammatical variants), as applied to an amino acid sequence of this invention, means a polypeptide that consists of the recited sequence (e.g., SEQ ID NO) and a total of fifty or less (e.g., 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1) additional amino acids at the amino terminal and/or carboxyl terminal ends of the recited sequence such that the function of the polypeptide is not materially altered. The total of fifty or less additional amino acids includes the total number of additional nucleotides on both ends added together.

The term “construct” refers to a recombinant genetic molecule including one or more isolated nucleic acid sequences from different sources. As used herein, the term “expression construct,” “recombinant construct” or “recombinant DNA construct” refers to any recombinant nucleic acid molecule such as a plasmid, cosmid, virus, autonomously replicating polynucleotide molecule, phage, or linear or circular single-stranded or double-stranded DNA or RNA nucleic acid molecule, derived from any source, capable of genomic integration or autonomous replication, comprising a nucleic acid molecule where one or more nucleic acid molecules have been operably linked. An “expression construct” generally includes at least a control sequence operably linked to a nucleotide sequence of interest. In this manner, for example, plant promoters in operable connection with the nucleotide sequences to be expressed are provided in expression constructs for expression in a plant, plant part, plant organ and/or plant cell. Methods are known for introducing constructs into a cell in such a manner that a transcribable polynucleotide molecule is transcribed into a functional mRNA molecule that is translated and therefore expressed as a protein product. Constructs may also be made to be capable of expressing inhibitory RNA molecules in order, for example, to inhibit translation of a specific RNA molecule of interest. For the practice of the present invention, conventional compositions and methods for preparing and using constructs and host cells are well known to one skilled in the art, see for example, Molecular Cloning: A Laboratory Manual, 3.sup.rd edition Volumes 1, 2, and 3. J. F. Sambrook, D. W. Russell, and N. Irwin, Cold Spring Harbor Laboratory Press, 2000.

By “corresponds to” or “corresponding to” is meant a nucleic acid sequence that displays substantial sequence identity to a reference nucleic acid sequence (e.g., at least about 70, 71, 72, 73, 74, 75, 76, 77, 78, 79, 80, 81, 82, 83, 84, 85, 86, 97, 88, 89, 90, 91, 92, 93, 94, 95, 96, 97, 98, 99% or even up to 100% sequence identity to all or a portion of the reference nucleic acid sequence) or an amino acid sequence that displays substantial sequence similarity or identity to a reference amino acid sequence (e.g., at least 70, 71, 72, 73, 74, 75, 76, 77, 78, 79, 80, 81, 82, 83, 84, 85, 86, 97, 88, 89, 90, 91, 92, 93, 94, 95, 96, 97, 98, 99% or even up to 100% sequence similarity or identity to all or a portion of the reference amino acid sequence).

As used herein, the terms “encode,” “encoding” and the like refer to the capacity of a nucleic acid to provide for another nucleic acid or a polypeptide. For example, a nucleic acid sequence is said to “encode” a polypeptide if it can be transcribed and/or translated to produce the polypeptide or if it can be processed into a form that can be transcribed and/or translated to produce the polypeptide. Such a nucleic acid sequence may include a coding sequence or both a coding sequence and a non-coding sequence. Thus, the terms “encode,” “encoding” and the like include an RNA product resulting from transcription of a DNA molecule, a protein resulting from translation of an RNA molecule, a protein resulting from transcription of a DNA molecule to form an RNA product and the subsequent translation of the RNA product, or a protein resulting from transcription of a DNA molecule to provide an RNA product, processing of the RNA product to provide a processed RNA product (e.g., mRNA) and the subsequent translation of the processed RNA product.

The term “endogenous” refers to any polynucleotide or polypeptide which is present and/or naturally expressed within a plant or a cell thereof. For example, an “endogenous” nucleic acid refers to a nucleic acid molecule or nucleotide sequence that is naturally found in the cell into which a construct of the invention is introduced.

The term “expression” with respect to a gene sequence refers to transcription of the gene and, as appropriate, translation of the resulting mRNA transcript to a protein. Thus, as will be clear from the context, expression of a coding sequence results from transcription and translation of the coding sequence. Conversely, expression of a non-coding sequence results from the transcription of the non-coding sequence.

As used herein, the terms “fragment” or “portion” when used in reference to a nucleic acid molecule or nucleotide sequence will be understood to mean a nucleic acid molecule or nucleotide sequence of reduced length relative to a reference nucleic acid molecule or nucleotide sequence and comprising, consisting essentially of and/or consisting of a nucleotide sequence of contiguous nucleotides identical or corresponding to the reference nucleic acid or nucleotide sequence. Such a nucleic acid fragment according to the invention may be, where appropriate, included in a larger polynucleotide of which it is a constituent.

As used herein, the term “gene” refers to a nucleic acid molecule capable of being used to produce mRNA, antisense RNA, siRNA, shRNA, miRNA, and the like. Genes may or may not be capable of being used to produce a functional protein. Genes can include both coding and non-coding regions (e.g., introns, regulatory elements, promoters, enhancers, termination sequences and 5′ and 3′ untranslated regions). A gene may be “isolated” by which is meant a nucleic acid molecule that is substantially or essentially free from components normally found in association with the nucleic acid molecule in its natural state. Such components include other cellular material, culture medium from recombinant production, and/or various chemicals used in chemically synthesizing the nucleic acid molecule.

“Genome” as used herein includes the nuclear and/or plastid genome, and therefore includes integration of the nucleic acid into, for example, the chloroplast genome.

The term “heterologous” as used herein with reference to nucleic acids refers to a nucleic acid molecule or nucleotide sequence that either originates from another species or is from the same species or organism but is modified from either its original form or the form primarily expressed in the cell. Thus, a nucleotide sequence derived from an organism or species different from that of the cell into which the nucleotide sequence is introduced, is heterologous with respect to that cell and the cell's descendants. In addition, a heterologous nucleotide sequence includes a nucleotide sequence derived from and inserted into the same natural, original cell type, but which is present in a non-natural state, e.g. present in a different copy number, and/or under the control of different regulatory sequences than that found in the native state of the nucleic acid molecule. The term “heterologous” when used with reference to portions of a nucleic acid indicates that the nucleic acid comprises two or more subsequences that are not found in the same relationship to each other in nature. For instance, a nucleic acid may be recombinantly produced, having two or more sequences from unrelated genes arranged to make a new functional nucleic acid, e.g., a nucleic acid encoding a protein from one source and a nucleic acid encoding a peptide sequence from another source. Similarly, a “heterologous” protein indicates that the protein comprises two or more subsequences that are not found in the same relationship to each other in nature (e.g., a fusion protein).

As used herein the term “homology” refers to the level of similarity between two or more nucleotide sequences and/or amino acid sequences in terms of percent of positional identity (i.e., sequence similarity or identity). Different nucleotide sequences or polypeptide sequences having homology are referred to herein as “homologs.” The term homolog includes homologous sequences from the same and other species and orthologous sequences from the same and other species. Homology also refers to the concept of similar functional properties among different nucleic acids, amino acids, and/or proteins.

Reference herein to “immuno-interactive” includes reference to any interaction, reaction, or other form of association between molecules and in particular where one of the molecules is, or mimics, a component of the immune system.

By “inactivation” is meant a genetic modification of a gene, including loss-of-function genetic modifications, which decreases, abrogates or otherwise inhibits the level or functional activity of an expression product of that gene.

“Introducing” in the context of a plant cell, plant part and/or plant organ means contacting a nucleic acid molecule with the plant, plant part, and/or plant cell in such a manner that the nucleic acid molecule gains access to the interior of the plant cell and/or a cell of the plant and/or plant part. Where more than one nucleic acid molecule is to be introduced these nucleic acid molecules can be assembled as part of a single polynucleotide or nucleic acid construct, or as separate polynucleotide or nucleic acid constructs, and can be located on the same or different nucleic acid constructs. Accordingly, these polynucleotides can be introduced into plant cells in a single transformation event, in separate transformation events, or, e.g., as part of a breeding protocol. Thus, the term “transformation” as used herein refers to the introduction of a heterologous nucleic acid into a cell. Transformation of a cell may be stable or transient. “Transient transformation” in the context of a polynucleotide means that a polynucleotide is introduced into the cell and does not integrate into the genome of the cell. By “stably introducing” or “stably introduced” in the context of a polynucleotide introduced into a cell, it is intended that the introduced polynucleotide is stably incorporated into the genome of the cell, and thus the cell is stably transformed with the polynucleotide. “Stable transformation” or “stably transformed” as used herein means that a nucleic acid molecule is introduced into a cell and integrates into the genome of the cell. As such, the integrated nucleic acid molecule is capable of being inherited by the progeny thereof, more particularly, by the progeny of multiple successive generations. Stable transformation as used herein can also refer to a nucleic acid molecule that is maintained extrachromosomally, for example, as a minichromosome.

An “isolated” nucleic acid molecule or nucleotide sequence or nucleic acid construct or double stranded RNA molecule of the present invention is generally free of nucleotide sequences that flank the nucleic acid of interest in the genomic DNA of the organism from which the nucleic acid was derived (such as coding sequences present at the 5′ or 3′ ends). However, the nucleic acid molecules of the present invention can include some additional bases or moieties that do not deleteriously or materially affect the basic structural and/or functional characteristics of the nucleic acid molecule. Thus, an “isolated nucleic acid molecule” or “isolated nucleotide sequence” is a nucleic acid molecule or nucleotide sequence that is not immediately contiguous with nucleotide sequences with which it is immediately contiguous (one on the 5′ end and one on the 3′ end) in the naturally occurring genome of the organism from which it is derived. Accordingly, in some embodiments, an isolated nucleic acid includes some or all of the 5′ non-coding (e.g., promoter) sequences that are immediately contiguous to a coding sequence. The term therefore includes, for example, a recombinant nucleic acid that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. It also includes a recombinant nucleic acid that is part of a hybrid nucleic acid molecule encoding an additional polypeptide or peptide sequence.

The term “isolated” can further refer to a nucleic acid molecule, nucleotide sequence, polypeptide, peptide or fragment that is substantially free of cellular material, viral material, and/or culture medium (e.g., when produced by recombinant DNA techniques), or chemical precursors or other chemicals (e.g., when chemically synthesized). Moreover, an “isolated fragment” is a fragment of a nucleic acid molecule, nucleotide sequence or polypeptide that is not naturally occurring as a fragment and would not be found as such in the natural state. “Isolated” does not mean that the preparation is technically pure (homogeneous), but it is sufficiently pure to provide the polypeptide or nucleic acid in a form in which it can be used for the intended purpose. Accordingly, “isolated” refers to a nucleic acid molecule, nucleotide sequence, polypeptide, peptide or fragment that is altered “by the hand of man” from the natural state; i.e., that, if it occurs in nature, it has been changed or removed from its original environment, or both. For example, a naturally occurring polynucleotide or a polypeptide naturally present in a living organism in its natural state is not “isolated,” but the same polynucleotide or polypeptide separated from the coexisting materials of its natural state is “isolated,” as the term is employed herein. For example, with respect to polynucleotides, the term isolated means that it is separated from the chromosome and/or cell in which it naturally occurs. A polynucleotide is also isolated if it is separated from the chromosome and/or cell in which it naturally occurs in and is then inserted into a genetic context, a chromosome and/or a cell in which it does not naturally occur. In representative embodiments of the invention, an “isolated” nucleic acid molecule, nucleotide sequence, and/or polypeptide is at least about 5%, 10%, 15%, 20%, 25%, 30%, 40%, 50%, 60%, 70%, 75%, 80%, 85%, 90%, 95%, 97%, 98%, 99% pure (w/w) or more. In other embodiments, an “isolated” nucleic acid, nucleotide sequence, and/or polypeptide indicates that at least about a 5-fold, 10-fold, 25-fold, 100-fold, 1000-fold, 10,000-fold, 100,000-fold or more enrichment of the nucleic acid (w/w) is achieved as compared with the starting material.

The term “isolated” when used in the context of an “isolated cell,” refers to a cell that has been removed from its natural environment, for example, as a part of an organ, tissue, or organism. For example, an isolated cell can be a cell in culture medium.

The term “loss-of-function,” is art recognized and, with respect to a gene or gene product, refers to mutations in a gene which ultimately decrease or otherwise inhibit the level or functional activity of an expression product of that gene. For example, a loss-of-function mutation to a gene of interest may be a point mutation, deletion or insertion of sequences in the coding sequence, intron sequence or 5′ or 3′ flanking sequences of the gene so as to, for example, (i) alter (e.g., decrease) the level gene expression, (ii) alter exon-splicing patterns, (iii) alter the activity of the encoded protein, or (iv) alter (decrease) the stability of the encoded protein.

The term, “microRNA” or “miRNAs” refer to small, noncoding RNA molecules that have been found in a diverse array of eukaryotes, including plants. miRNA precursors share a characteristic secondary structure, forming short ‘hairpin’ RNAs. The term “miRNA” includes processed sequences as well as corresponding long primary transcripts (pri-miRNAs) and processed precursors (pre-miRNAs). Genetic and biochemical studies have indicated that miRNAs are processed to their mature forms by Dicer, an RNAse III family nuclease, and function through RNA-mediated interference (RNAi) and related pathways to regulate the expression of target genes (Hannon (2002) Nature 418, 244-251; Pasquinelli, et al. (2002) Annu. Rev. Cell. Dev. Biol. 18, 495-513). miRNAs may be configured to permit experimental manipulation of gene expression in cells as synthetic silencing triggers ‘short hairpin RNAs’ (shRNAs) (Paddison et al. (2002) Cancer Cell 2, 17-23). Silencing by shRNAs involves the RNAi machinery and correlates with the production of small interfering RNAs (siRNAs), which are a signature of RNAi.

The term “non-coding” refers to sequences of nucleic acid molecules that do not encode part or all of an expressed protein. Non-coding sequences include but are not limited to introns, enhancers, promoter regions, 3′ untranslated regions, and 5′ untranslated regions. Thus, the term “5′-non-coding region” shall be taken in its broadest context to include all nucleotide sequences which are derived from the upstream region of a gene. Such regions may include an intron, e.g., an intron. As used herein, the term “3′ non-coding region” refers to nucleic acid sequences located downstream of a coding sequence and include polyadenylation recognition sequences (normally limited to eukaryotes) and other sequences encoding regulatory signals capable of affecting mRNA processing or gene expression. The polyadenylation signal (normally limited to eukaryotes) is usually characterized by affecting the addition of polyadenylic acid tracts to the 3′ end of the mRNA precursor.

As used herein, the term “nucleotide sequence” refers to a heteropolymer of nucleotides or the sequence of these nucleotides from the 5′ to 3′ end of a nucleic acid molecule and includes DNA or RNA molecules, including cDNA, a DNA fragment, genomic DNA, synthetic (e.g., chemically synthesized) DNA, plasmid DNA, mRNA, and anti-sense RNA, any of which can be single stranded or double stranded. The terms “nucleotide sequence” “nucleic acid,” “nucleic acid molecule,” “oligonucleotide” and “polynucleotide” are also used interchangeably herein to refer to a heteropolymer of nucleotides. Nucleic acid sequences provided herein are presented herein in the 5′ to 3′ direction, from left to right and are represented using the standard code for representing the nucleotide characters as set forth in the U.S. sequence rules, 37 CFR 1.821-1.825 and the World Intellectual Property Organization (WIPO) Standard ST.25.

The term “operably connected” or “operably linked” as used herein refers to a juxtaposition wherein the components so described are in a relationship permitting them to function in their intended manner. For example, a control sequence (e.g., a promoter) “operably linked” to a coding sequence refers to positioning and/or orientation of the control sequence relative to the coding sequence to permit expression of the coding sequence under conditions compatible with the control sequence. The control sequences need not be contiguous with the nucleotide sequence of interest, so long as they function to direct the expression thereof. Thus, for example, intervening untranslated, yet transcribed, sequences can be present between a promoter and a coding sequence, and the promoter sequence can still be considered “operably linked” to the coding sequence. Likewise, “operably connecting” a cis-acting sequence to a promoter encompasses positioning and/or orientation of the cis-acting sequence relative to the promoter so that (1) the cis-acting sequence regulates (e.g., inhibits, abrogates, stimulates or enhances) promoter activity.

As used herein, “plant” means any plant and thus includes, for example, angiosperms (monocots and dicots), gymnosperms, bryophytes, ferns and/or fern allies. Non-limiting examples of sucrose-accumulating crop plants of the present invention include monocotyledonous plants, illustrative examples of which include sugarcane, corn, barley, rye, oats, wheat, rice, flax, millet, sorghum, grasses, banana, onion, asparagus, lily, coconut, and the like, as well as dicotyledonous plants such as but not limited to.

As used herein, the term “plant part” includes but is not limited to embryos, pollen, ovules, seeds, leaves, flowers, branches, fruit, kernels, ears, cobs, husks, stalks, roots, root tips, anthers, plant cells including plant cells that are intact in plants and/or parts of plants, plant protoplasts, plant tissues, plant cell tissue cultures, plant calli, plant clumps, and the like.

As used herein, “plant cell” refers to a structural and physiological unit of the plant, which comprises a cell wall and also may refer to a protoplast. A plant cell of the present invention can be in the form of an isolated single cell or can be a cultured cell or can be a part of a higher-organized unit such as, for example, a plant tissue or a plant organ.

The term “plant organ” refers to plant tissue or group of tissues that constitute a morphologically and functionally distinct part of a plant.

As used herein, the terms “polynucleotide,” “polynucleotide sequence,” “nucleotide sequence,” “nucleic acid,” “nucleic acid molecule,” “nucleic acid sequence and the like refer to RNA or DNA that is linear or branched, single or double stranded, or a hybrid thereof. The term also encompasses RNA/DNA hybrids. The term typically refers to polymeric form of nucleotides of at least 10 bases in length, either ribonucleotides or deoxynucleotides or a modified form of either type of nucleotide. The term includes single and double stranded forms of RNA or DNA. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

“Polypeptide,” “peptide,” “protein” and “proteinaceous molecule” are used interchangeably herein to refer to molecules comprising or consisting of a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers. This term also includes within its scope two or more complementing or interactive polypeptides comprising different parts or portions (e.g., polypeptide domains, polypeptide chains etc.) of a luciferase polypeptide of the present invention, wherein the individual complementing polypeptides together reconstitute the activity of the different parts or portions to form a functional luciferase polypeptide. Such complementing polypeptides are used routinely in protein complementation assays, which are well known to persons skilled in the art.

As used herein, the term “post-transcriptional gene silencing” (PTGS) refers to a form of gene silencing in which the inhibitory mechanism occurs after transcription. This can result in either decreased steady-state level of a specific RNA target or inhibition of translation (Tuschl et al. (2001) ChemBiochem 2: 239-245). In the literature, the terms RNA interference (RNAi) and posttranscriptional co-suppression are often used to indicate posttranscriptional gene silencing.

By “primer” is meant an oligonucleotide which, when paired with a strand of DNA, is capable of initiating the synthesis of a primer extension product in the presence of a suitable polymerizing agent. The primer is preferably single-stranded for maximum efficiency in amplification but can alternatively be double-stranded. A primer must be sufficiently long to prime the synthesis of extension products in the presence of the polymerization agent. The length of the primer depends on many factors, including application, temperature to be employed, template reaction conditions, other reagents, and source of primers. For example, depending on the complexity of the target sequence, the primer may be at least about 5, 6, 7, 8, 9, 10, 11, 12, 13, 14, 15, 16, 17, 18, 19, 20, 21, 22, 23, 24, 25, 26, 27, 28, 29, 30, 35, 40, 50, 75, 100, 150, 200, 300, 400, 500, to one base shorter in length than the template sequence at the 3′ end of the primer to allow extension of a nucleic acid chain, though the 5′ end of the primer may extend in length beyond the 3′ end of the template sequence. In certain embodiments, primers can be large polynucleotides, such as from about 35 nucleotides to several kilobases or more. Primers can be selected to be “substantially complementary” to the sequence on the template to which it is designed to hybridize and serve as a site for the initiation of synthesis. By “substantially complementary”, it is meant that the primer is sufficiently complementary to hybridize with a target polynucleotide. Desirably, the primer contains no mismatches with the template to which it is designed to hybridize but this is not essential. For example, non-complementary nucleotide residues can be attached to the 5′ end of the primer, with the remainder of the primer sequence being complementary to the template. Alternatively, non-complementary nucleotide residues or a stretch of non-complementary nucleotide residues can be interspersed into a primer, provided that the primer sequence has sufficient complementarity with the sequence of the template to hybridize therewith and thereby form a template for synthesis of the extension product of the primer.

The term “probe,” as used herein, refers to a molecule that binds to a specific sequence or sub-sequence or other moiety of another molecule. Unless otherwise indicated, the term “probe” typically refers to a nucleic acid probe that binds to another nucleic acid molecule, often called the “target nucleic acid molecule”, through complementary base pairing. Probes can bind target nucleic acid molecules lacking complete sequence complementarity with the probe, depending on the stringency of the hybridization conditions. Probes can be labeled directly or indirectly and include primers within their scope.

As used herein, the term “promoter” refers to a region of a nucleotide sequence that incorporates the necessary signals for the expression of a coding sequence operably associated with the promoter. This may include sequences to which an RNA polymerase binds, but is not limited to such sequences and can include regions to which other regulatory proteins bind, together with regions involved in the control of protein translation and can also include coding sequences. Furthermore, a “promoter” of this invention is a promoter (e.g., a nucleotide sequence) capable of initiating transcription of a nucleic acid molecule in a cell of a plant.

“Promoter activity” refers to the ability of a promoter to drive expression of a nucleic acid sequence operably linked to the promoter. Promoter activity of a sequence can be assessed by operably linking the sequence to a reporter gene, and determining expression of the reporter.

The term “recombinant polynucleotide” refers to a polynucleotide that has been altered, rearranged, or modified by genetic engineering. Examples include any cloned polynucleotide, or polynucleotides, that are linked or joined to heterologous sequences. However, it shall be understood that the term “recombinant” does not refer to alterations of polynucleotides that result from naturally occurring events, such as spontaneous mutations, or from non-spontaneous mutagenesis followed by selective breeding.

As used herein, the terms “RNA interference” and “RNAi” refer to a sequence-specific process by which a target molecule (e.g., a target gene, protein or RNA) is down-regulated via down-regulation of expression. Without being bound to a specific mechanism, as currently understood by those of skill in the art, RNAi involves degradation of RNA molecules, e.g., mRNA molecules within a cell, catalyzed by an enzymatic, RNA-induced silencing complex (RISC). RNAi occurs in cells naturally to remove foreign RNAs (e.g., viral RNAs) triggered by dsRNA fragments cleaved from longer dsRNA which direct the degradative mechanism to other RNA sequences having closely homologous sequences. As practiced as a technology, RNAi can be initiated by human intervention to reduce or even silence the expression of target genes using either exogenously synthesized dsRNA or dsRNA transcribed in the cell (e.g., synthesized as a sequence that forms a short hairpin structure).

As used herein, the terms “small interfering RNA” and “short interfering RNA” (“siRNA”) refer to a short RNA molecule, generally a double-stranded RNA molecule about 10-50 nucleotides in length (the term “nucleotides” including nucleotide analogs), preferably between about 15-25 nucleotides in length. In most cases, the siRNA is 17, 18, 19, 20, 21, 22, 23, 24, or 25 nucleotides in length. Such siRNA can have overhanging ends (e.g., 3′-overhangs of 1, 2, or 3 nucleotides (or nucleotide analogs). Such siRNA can mediate RNA interference.

As used in connection with the present invention, the term “shRNA” refers to an RNA molecule having a stem-loop structure. The stem-loop structure includes two mutually complementary sequences, where the respective orientations and the degree of complementarity allow base pairing between the two sequences. The mutually complementary sequences are linked by a loop region, the loop resulting from a lack of base pairing between nucleotides (or nucleotide analogs) within the loop region.

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software. Useful methods for determining sequence identity are also disclosed in Guide to Huge Computers (Martin J. Bishop, ed., Academic Press, San Diego (1994)), and Carillo et al. (Applied Math 48:1073 (1988)). More particularly, preferred computer programs for determining sequence identity include but are not limited to the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; (Altschul et al., J. Mol. Biol. 215:403-410 (1990)); version 2.0 or higher of BLAST programs allows the introduction of gaps (deletions and insertions) into alignments; for peptide sequence BLASTX can be used to determine sequence identity; and for polynucleotide sequence BLASTN can be used to determine sequence identity.

“Similarity” refers to the percentage number of amino acids that are identical or constitute conservative substitutions as defined in Table A below. Similarity may be determined using sequence comparison programs such as GAP (Deveraux et al. 1984, Nucleic Acids Research 12: 387-395). In this way, sequences of a similar or substantially different length to those cited herein might be compared by insertion of gaps into the alignment, such gaps being determined, for example, by the comparison algorithm used by GAP.

TABLE A
Exemplary Conservative Amino Acid Substitutions

| Original Residue | Exemplary Substitutions |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile, |
| Phe | Met, Leu, Tyr |
| Ser | Thr |
| Thr | Ser |
| Trp | Tyr |
| Tyr | Trp, Phe |
| Val | Ile, Leu |

Terms used to describe sequence relationships between two or more polynucleotides or polypeptides include “reference sequence,” “comparison window”, “sequence identity,” “percentage of sequence identity” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., “Current Protocols in Molecular Biology”, John Wiley & Sons Inc, 1994-1998, Chapter 15.

“Stem-specific promoter” as used herein refers to a promoter that transcribes an operably connected nucleic acid sequence in a way that transcription of the nucleic acid sequence in plant stem tissues contribute to more than 80%, 85%, 90%, 95%, 99% of the entire quantity of the RNA transcribed from the nucleic acid sequence in the entire plant during any of its developmental stages.

“Stem-preferential promoter” in the context of this invention refers to a promoter that transcribes an operably connected nucleic acid sequence in a way that transcription of the nucleic acid sequence in plant stem tissues contribute to more than 50%, preferably more than 70%, more preferably more than 80% of the entire quantity of the RNA transcribed from said nucleic acid sequence in the entire plant during any of its developmental stages.

The term “sucrose derivative” is used herein in its broadest sense and includes: monosaccharides (aldoses and ketoses) comprising compounds with the empirical formula (CH₂O)ₙ where n=3 or some larger number; including tetroses (e.g., erythrose, threose, erythrulose), pentoses (e.g., ribose, arabinose, xylose, lyxose, ribulose, xylulose), hexoses (e.g., allose, altrose, glucose, mannose, gulose, idose, galactose, talose, psicose, fructose, sorbose, tagatose), and longer molecules such as sedoheptulose or mannoheptulose; oligosaccharides formed by linking together of several monosaccharide units through glycosidic bonds; including disaccharides (e.g., maltose, lactose, gentibiose, melibiose, trehalose, sophorose, primoverose, rutinose, sucrose, isomaltulose, trehalulose, turanose, maltulose, leucrose) and longer oligomers such as raffinose, melezitose, bemisiose or stachyose; sugar alcohols (e.g., erythritol, ribitol, mannitol, sorbitol); sugar acids (e.g., gluconic acid, glucaric acid, glucuronic acid); amino sugars (e.g., glucosamine, galactosamine); and other variants such as deoxy sugars, methyl sugars, sugar phosphates and NDP-sugars (e.g., ADP, UDP, GDP, TDP, etc.), some of which may be converted into sugars or other sugar derivatives described above by the action of plant metabolic pathways.

As used herein, the terms “transformed” and “transgenic” refer to any plant, plant cell, callus, plant tissue, or plant part that contains all or part of at least one isolated or recombinant (e.g., heterologous) polynucleotide. In some embodiments, all or part of the isolated or recombinant polynucleotide is stably integrated into a chromosome or stable extra-chromosomal element, so that it is passed on to successive generations.

The term “transgene” as used herein, refers to any nucleotide sequence used in the transformation of a plant, animal, or other organism. Thus, a transgene can be a coding sequence, a non-coding sequence, a cDNA, a gene or fragment or portion thereof, a genomic sequence, a regulatory element and the like. A “transgenic” organism, such as a transgenic plant, transgenic microorganism, or transgenic animal, is an organism into which a transgene has been delivered or introduced and the transgene can be expressed in the transgenic organism to produce a product, the presence of which can impart an effect and/or a phenotype in the organism.

As used herein, the term “5′ untranslated region” or “5′ UTR” refers to a sequence located 3′ to promoter region and 5′ of the downstream coding region. Thus, such a sequence, while transcribed, is upstream (i.e., 5′) of the translation initiation codon and therefore is generally not translated into a portion of the polypeptide product.

The term “3′ untranslated region” or “3′ UTR” refers to a nucleotide sequence downstream (i.e., 3′) of a coding sequence. It extends from the first nucleotide after the stop codon of a coding sequence to just before the poly(A) tail of the corresponding transcribed mRNA. The 3′ UTR may contain sequences that regulate translation efficiency, mRNA stability, mRNA targeting and/or polyadenylation.

The terms “wild-type,” “natural,” “native” and the like with respect to an organism, polypeptide, or nucleic acid sequence, that the organism polypeptide, or nucleic acid sequence is naturally occurring or available in at least one naturally occurring organism which is not changed, mutated, or otherwise manipulated by man.

As used herein, underscoring or italicizing the name of a gene shall indicate the gene, in contrast to its protein product, which is indicated in the absence of any underscoring or italicizing. For example, “SUS2” shall mean a SUS2 gene or gene subfamily, whereas “SUS2” shall indicate the protein product of a “SUS2” gene or gene subfamily.

Each embodiment described herein is to be applied mutatis mutandis to each and every embodiment unless specifically stated otherwise.

### 2. SUS2 Nucleic Acids

The present invention is based in part on the identification of five SUS gene subfamilies in the sucrose-accumulating crop plants sorghum and sugarcane and the determination that inhibiting expression of a specific one of these subfamilies (SUS2), suitably in a specific tissue and/or developmental stage, is effective for significantly increasing the concentration or yield of sucrose or sucrose derivatives in harvestable plant storage organs of sucrose-accumulating crop plants.

It will be apparent that the SUS2 nucleic acid sequences disclosed herein (e.g., SEQ ID NO: 1 and 3) will find utility in a variety of applications, examples of which include constructing nucleic acid constructs for expressing SUS2 inhibitory RNA molecules, or for producing recombinant SUS2 polypeptides, which can be used for example for producing SUS2 antibodies.

The SUS2 nucleic acid sequences may in turn be used to design specific oligonucleotide probes or primers for detecting SUS2 nucleic acid sequences, or for identifying SUS2 homologs in sucrose-accumulating crop plants. Such probes or primers may be of any length that would specifically hybridize to the identified marker gene sequences and may be at least about 10, 11, 12, 13, 14, 15, 16, 17, 18, 19, 20, 21, 22, 23, 24, 25, 26, 27, 28, 29, 30, 35, 40, 50, 75, 100, 150, 200, 300, 400, 500, 600, 700, 800, 900, 1000, 1200, 1400, 1600, 1800, 2000, 2200 nucleotides in length and in the case of probes, up to the full length of the sequences of the SUS2 gene identified herein.

Probes may also include additional sequence at their 5′ and/or 3′ ends so that they extent beyond the target sequence with which they hybridize. The present invention thus also encompasses portions of the disclosed SUS2 nucleic acid sequences. These portions may comprise coding sequences or non coding sequences corresponding to the disclosed SUS2 nucleic acid sequences. The portions may range from at least about 10, 11, 12, 13, 14, 15, 16, 17, 18, 19, 20, 21, 22, 23, 24, 25, 26, 27, 28, 29, 30, 35, 40, 50, 75, 100, 150, 200, 300, 400, 500, 600, 700, 800, 900, 1000, 1200, 1400, 1600, 1800, 2000, 2200 nucleotides, or up to the full-length SUS2 nucleic acid sequence disclosed herein.

The SUS2 nucleic acid sequences may also be used to identify and isolate full-length gene sequences, including regulatory elements for gene expression, from genomic DNA libraries, which are suitably but not exclusively of sucrose-accumulating crop plant origin. The SUS2 nucleic acid sequences identified in the present disclosure may be used as hybridization probes to screen genomic DNA libraries by conventional techniques. Once partial genomic clones have been identified, full-length genes may be isolated by “chromosomal walking” (also called “overlap hybridization”) using, for example, the method disclosed by Chinault & Carbon (1979, Gene 5: 111-126). Once a partial genomic clone has been isolated using a cDNA hybridization probe, non-repetitive segments at or near the ends of the partial genomic clone may be used as hybridization probes in further genomic library screening, ultimately allowing isolation of entire gene sequences belonging to the SUS2 gene subfamily. It will be recognized that full-length genes may be obtained using the full-length or partial cDNA sequences or short expressed sequence tags (ESTs) described in this disclosure using standard techniques as disclosed for example by Sambrook, et al. (MOLECULAR CLONING. A LABORATORY MANUAL (Cold Spring Harbor Press, 1989) and Ausubel et al., (CURRENT PROTOCOLS IN MOLECULAR BIOLOGY, John Wiley & Sons, Inc. 1994). In addition, the disclosed sequences may be used to identify and isolate full-length cDNA sequences using standard techniques as disclosed, for example, in the above-referenced texts. Sequences identified and isolated by such means are part of the invention.

The present invention also encompasses isolated nucleic acids that are variants of the disclosed SUS2 nucleic acids or that are hybridizable to these nucleic acids. Nucleic acid variants can be naturally-occurring, such as allelic variants (same locus), homologs (different locus), and orthologs (different organism) or can be non naturally-occurring. Naturally occurring variants such as these can be identified with the use of well-known molecular biology techniques, as, for example, with polymerase chain reaction (PCR) and hybridization techniques as known in the art. Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product). For nucleotide sequences, conservative variants include those sequences that, because of the degeneracy of the genetic code, encode the amino acid sequence of the disclosed SUS2 polypeptide of the invention. Variant nucleotide sequences also include synthetically derived nucleotide sequences, such as those generated, for example, by using site-directed mutagenesis but which still encode a SUS2 polypeptide of the invention. Generally, variants of a SUS2 nucleotide sequence of the invention will have at least about 70%, 75%, 80%, 85%, desirably at least about 90%, 91%, 92%, 93%, 94%, 95%, 96%, 97%, 98%, 99% sequence identity to that particular nucleotide sequence as determined by sequence alignment programs described elsewhere herein using default parameters.

The SUS2 nucleic acid sequences of the invention can be used to isolate corresponding sequences and alleles from other sucrose-accumulating plants. Methods are readily available in the art for the hybridization of nucleic acid sequences. For example, coding sequences from other sucrose-accumulating plants may be isolated according to well known techniques based on their sequence identity with the coding sequences set forth herein. In these techniques all or part of the known coding sequence is used as a probe which selectively hybridizes to another SUS2 coding sequences present in a population of cloned genomic DNA fragments or cDNA fragments (i.e., genomic or cDNA libraries) from a chosen sucrose-accumulating plant. Accordingly, the present invention also contemplates nucleic acid molecules that hybridize to the disclosed SUS2 nucleic acid sequences, or to their complements, under stringency conditions described below. As used herein, the term “hybridizes under low stringency, medium stringency, high stringency, or very high stringency conditions” describes conditions for hybridization and washing. Guidance for performing hybridization reactions can be found in Ausubel et al., (1998, supra), Sections 6.3.1-6.3.6. Aqueous and non-aqueous methods are described in that reference and either can be used. Reference herein to low stringency conditions include and encompass from at least about 1% v/v to at least about 15% v/v formamide and from at least about 1 M to at least about 2 M salt for hybridization at 42° C., and at least about 1 M to at least about 2 M salt for washing at 42° C. Low stringency conditions also may include 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at room temperature. One embodiment of low stringency conditions includes hybridization in 6×sodium chloride/sodium citrate (SSC) at about 45° C., followed by two washes in 0.2×SSC, 0.1% SDS at least at 50° C. (the temperature of the washes can be increased to 55° C. for low stringency conditions). Medium stringency conditions include and encompass from at least about 16% v/v to at least about 30% v/v formamide and from at least about 0.5 M to at least about 0.9 M salt for hybridization at 42° C., and at least about 0.1 M to at least about 0.2 M salt for washing at 55° C. Medium stringency conditions also may include 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at 60-65° C. One embodiment of medium stringency conditions includes hybridizing in 6×SSC at about 45° C., followed by one or more washes in 0.2×SSC, 0.1% SDS at 60° C. High stringency conditions include and encompass from at least about 31% v/v to at least about 50% v/v formamide and from about 0.01 M to about 0.15 M salt for hybridization at 42° C., and about 0.01 M to about 0.02 M salt for washing at 55° C. High stringency conditions also may include 1% BSA, 1 mM EDTA, 0.5 M NaHPO₄ (pH 7.2), 7% SDS for hybridization at 65° C., and (i) 0.2×SSC, 0.1% SDS; or (ii) 0.5% BSA, 1 mM EDTA, 40 mM NaHPO₄ (pH 7.2), 1% SDS for washing at a temperature in excess of 65° C. One embodiment of high stringency conditions includes hybridizing in 6×SSC at about 45° C., followed by one or more washes in 0.2×SSC, 0.1% SDS at 65° C.

In certain embodiments, a SUS2 nucleic acid sequence hybridizes to a disclosed SUS2 nucleotide sequence under very high stringency conditions. One embodiment of very high stringency conditions includes hybridizing 0.5 M sodium phosphate, 7% SDS at 65° C., followed by one or more washes at 0.2×SSC, 1% SDS at 65° C.

Other stringency conditions are well known in the art and a skilled addressee will recognize that various factors can be manipulated to optimize the specificity of the hybridization. Optimization of the stringency of the final washes can serve to ensure a high degree of hybridization. For detailed examples, see Ausubel et al., supra at pages 2.10.1 to 2.10.16 and Sambrook et al. (1989, supra) at sections 1.101 to 1.104.

While stringent washes are typically carried out at temperatures from about 42° C. to 68° C., one skilled in the art will appreciate that other temperatures may be suitable for stringent conditions. Maximum hybridization rate typically occurs at about 20° C. to 25° C. below the Tₘ for formation of a DNA-DNA hybrid. It is well known in the art that the Tₘ is the melting temperature, or temperature at which two complementary polynucleotide sequences dissociate. Methods for estimating Tₘ are well known in the art (see Ausubel et al., supra at page 2.10.8). In general, the Tₘ of a perfectly matched duplex of DNA may be predicted as an approximation by the formula:

Tₘ=81.5+16.6(log₁₀M)+0.41(% G+C)−0.63(% formamide)−(600/length)

wherein: M is the concentration of Nat, preferably in the range of 0.01 molar to 0.4 molar; % G+C is the sum of guanosine and cytosine bases as a percentage of the total number of bases, within the range between 30% and 75% G+C; % formamide is the percent formamide concentration by volume; length is the number of base pairs in the DNA duplex. The Tₘ of a duplex DNA decreases by approximately 1° C. with every increase of 1% in the number of randomly mismatched base pairs. Washing is generally carried out at Tₘ —15° C. for high stringency, or Tₘ —30° C. for moderate stringency.

In one example of a hybridization procedure, a membrane (e.g., a nitrocellulose membrane or a nylon membrane) containing immobilized DNA is hybridized overnight at 42° C. in a hybridization buffer (50% deionised formamide, 5×SSC, 5×Denhardt's solution (0.1% Ficoll, 0.1% polyvinylpyrollidone and 0.1% bovine serum albumin), 0.1% SDS and 200 mg/mL denatured salmon sperm DNA) containing labeled probe. The membrane is then subjected to two sequential medium stringency washes (i.e., 2×SSC, 0.1% SDS for 15 min at 45° C., followed by 2×SSC, 0.1% SDS for 15 min at 50° C.), followed by two sequential higher stringency washes (i.e., 0.2×SSC, 0.1% SDS for 12 min at 55° C. followed by 0.2×SSC and 0.1% SDS solution for 12 min at 65-68° C.

### 3. SUS2 Polypeptides

The present invention also contemplates full-length SUS2 polypeptides, which comprise for example the amino acid sequence set forth in SEQ ID NO:2 or variants thereof, produced by sucrose-accumulating crop plants as well as their fragment, which are referred to collectively herein as “SUS2 polypeptides.” Fragments of full-length SUS2 polypeptides include portions with immuno-interactive activity of at least about 6, 8, 10, 12, 14, 16, 18, 20, 25, 30, 40, 50, 60 amino acid residues in length. For example, immuno-interactive fragments contemplated by the present invention are at least 6 and desirably at least 8 amino acid residues in length, which can elicit an immune response in an animal for the production of antibodies that are immuno-interactive with a SUS2 polypeptide of the invention. Such antibodies can be used to screen the same or other sucrose-accumulating crop plants, for structurally and/or functionally related SUS2 polypeptides.

The present invention also contemplates variant SUS2 polypeptides. “Variant” polypeptides include proteins derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Variant proteins encompassed by the present invention may be biologically active, that is, they continue to possess the desired biological activity of the native protein. Such variants may result from, for example, genetic polymorphism or from human manipulation.

Variant SUS2 polypeptides may contain conservative amino acid substitutions at various locations along their sequence, as compared to the parent SUS2 amino acid sequence. A “conservative amino acid substitution” is one in which the amino acid residue is replaced with an amino acid residue having a similar side chain. Families of amino acid residues having similar side chains have been defined in the art, which can be generally sub-classified as follows:

Acidic: The residue has a negative charge due to loss of H ion at physiological pH and the residue is attracted by aqueous solution so as to seek the surface positions in the conformation of a peptide in which it is contained when the peptide is in aqueous medium at physiological pH. Amino acids having an acidic side chain include glutamic acid and aspartic acid.

Basic: The residue has a positive charge due to association with H ion at physiological pH or within one or two pH units thereof (e.g., histidine) and the residue is attracted by aqueous solution so as to seek the surface positions in the conformation of a peptide in which it is contained when the peptide is in aqueous medium at physiological pH. Amino acids having a basic side chain include arginine, lysine and histidine.

Charged: The residues are charged at physiological pH and, therefore, include amino acids having acidic or basic side chains (i.e., glutamic acid, aspartic acid, arginine, lysine and histidine).

Hydrophobic: The residues are not charged at physiological pH and the residue is repelled by aqueous solution so as to seek the inner positions in the conformation of a peptide in which it is contained when the peptide is in aqueous medium. Amino acids having a hydrophobic side chain include tyrosine, valine, isoleucine, leucine, methionine, phenylalanine and tryptophan.

Neutral/polar: The residues are not charged at physiological pH, but the residue is not sufficiently repelled by aqueous solutions so that it would seek inner positions in the conformation of a peptide in which it is contained when the peptide is in aqueous medium. Amino acids having a neutral/polar side chain include asparagine, glutamine, cysteine, histidine, serine and threonine.

This description also characterizes certain amino acids as “small” since their side chains are not sufficiently large, even if polar groups are lacking, to confer hydrophobicity. With the exception of proline, “small” amino acids are those with four carbons or less when at least one polar group is on the side chain and three carbons or less when not. Amino acids having a small side chain include glycine, serine, alanine and threonine. The gene-encoded secondary amino acid proline is a special case due to its known effects on the secondary conformation of peptide chains. The structure of proline differs from all the other naturally-occurring amino acids in that its side chain is bonded to the nitrogen of the α-amino group, as well as the α-carbon. Several amino acid similarity matrices (e.g., PAM120 matrix and PAM250 matrix as disclosed for example by Dayhoff et al. (1978) A model of evolutionary change in proteins. Matrices for determining distance relationships In M. O. Dayhoff, (ed.), Atlas of protein sequence and structure, Vol. 5, pp. 345-358, National Biomedical Research Foundation, Washington D.C.; and by Gonnet et al., 1992, Science 256(5062): 144301445), however, include proline in the same group as glycine, serine, alanine and threonine. Accordingly, for the purposes of the present invention, proline is classified as a “small” amino acid.

The degree of attraction or repulsion required for classification as polar or nonpolar is arbitrary and, therefore, amino acids specifically contemplated by the invention have been classified as one or the other. Most amino acids not specifically named can be classified on the basis of known behavior.

Amino acid residues can be further sub-classified as cyclic or noncyclic, and aromatic or nonaromatic, self-explanatory classifications with respect to the side-chain substituent groups of the residues, and as small or large. The residue is considered small if it contains a total of four carbon atoms or less, inclusive of the carboxyl carbon, provided an additional polar substituent is present; three or less if not. Small residues are, of course, always nonaromatic. Dependent on their structural properties, amino acid residues may fall in two or more classes. For the naturally-occurring protein amino acids, sub-classification according to the this scheme is presented in the Table B.

TABLE B
Amino acid sub-classification

| Sub-classes | Amino acids |
|---|---|
| Acidic | Aspartic acid, Glutamic acid |
| Basic | Noncyclic: Arginine, Lysine; Cyclic: Histidine |
| Charged | Aspartic acid, Glutamic acid, Arginine, Lysine, |
| | Histidine |
| Small | Glycine, Serine, Alanine, Threonine, Proline |
| Polar/neutral | Asparagine, Histidine, Glutamine, Cysteine, |
| | Serine, Threonine |
| Polar/large | Asparagine, Glutamine |
| Hydrophobic | Tyrosine, Valine, Isoleucine, Leucine, |
| | Methionine, Phenylalanine, Tryptophan |
| Aromatic | Tryptophan, Tyrosine, Phenylalanine |
| Residues that influence | Glycine and Proline |
| chain orientation | |

Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting variant polypeptide. Whether an amino acid change results in a functional SUS2 polypeptide can readily be determined by assaying sucrose synthase activity using standard techniques in the art and disclosed herein. Conservative substitutions are shown in Table C below under the heading of exemplary substitutions. More preferred substitutions are shown under the heading of preferred substitutions. Amino acid substitutions falling within the scope of the invention, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. After the substitutions are introduced, the variants are screened for biological activity.

TABLE C
Exemplary and Preferred Amino Acid Substitutions

| Original | | Preferred |
|---|---|---|
| Residue | Exemplary Substitutions | Substitutions |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn, His, Lys, | Asn |
| Glu | Asp, Lys | Asp |
| Gly | Pro | Pro |
| His | Asn, Gln, Lys, Arg | Arg |
| Ile | Leu, Val, Met, Ala, Phe, Norleu | Leu |
| Leu | Norleu, Ile, Val, Met, Ala, Phe | Ile |
| Lys | Arg, Gln, Asn | Arg |
| Met | Leu, Ile, Phe | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Norleu | Leu |

Alternatively, similar amino acids for making conservative substitutions can be grouped into three categories based on the identity of the side chains. The first group includes glutamic acid, aspartic acid, arginine, lysine, histidine, which all have charged side chains; the second group includes glycine, serine, threonine, cysteine, tyrosine, glutamine, asparagine; and the third group includes leucine, isoleucine, valine, alanine, proline, phenylalanine, tryptophan, methionine, as described in Zubay, G., Biochemistry, third edition, Wm.C. Brown Publishers (1993).

In general, variants will display at least about 70%, 75%, 80%, 85%, desirably at least about 90%, 91%, 92%, 93%, 94%, 95%, 96%, 97%, 98%, 99% similarity to a disclosed SUS2 polypeptide sequence. Desirably, variants will have at least 70%, 75%, 80%, 85%, desirably at least about 90%, 91%, 92%, 93%, 94%, 95%, 96%, 97%, 98%, 99% sequence identity to a disclosed SUS2 polypeptide. Moreover, sequences differing from the native or parent sequences by the addition, deletion, or substitution of 1, 2, 3, 4, 5, 6, 7, 8, 9, 10, 11, 12, 13, 14, 15, 16, 17, 18, 19, 20, 30, 40, 50, 60, 70, 80, 90, 100, 150, 200, 300, 500 or more amino acids but which retain the properties of the disclosed SUS2 polypeptide are contemplated. A variant of a disclosed SUS2 polypeptide of the invention may differ from that protein generally by as much 200, 100, 50 or 20 amino acid residues or suitably by as few as 1-15 amino acid residues, as few as 1-10, such as 6-10, as few as 5, as few as 4, 3, 2, or even 1 amino acid residue.

SUS2 polypeptides may be prepared by any suitable procedure known to those of skill in the art. For example, the polypeptides may be prepared by a procedure including the steps of: (a) preparing a chimeric construct comprising a SUS2 nucleotide sequence which encodes at least a portion of a SUS2 polypeptide selected from a disclosed SUS2 polypeptide or a variant thereof, and which is operably linked to a regulatory element; (b) introducing the chimeric construct into a host cell; (c) culturing the host cell to express the SUS2 polypeptide; and (d) isolating the SUS2 polypeptide from the host cell. In illustrative examples, the nucleotide sequence encodes at least a portion of the sequence set forth in SEQ ID NO:2, or a variant thereof.

Recombinant SUS2 polypeptides can be conveniently prepared using standard protocols as described for example in Sambrook, et al., (1989, supra), in particular Sections 16 and 17; Ausubel et al., (1994, supra), in particular Chapters 10 and 16; and Coligan et al., CURRENT PROTOCOLS IN PROTEIN SCIENCE (John Wiley & Sons, Inc. 1995-1997), in particular Chapters 1, 5 and 6. Alternatively, SUS2 polypeptides, including their fragments, may be synthesized by chemical synthesis, e.g., using solution synthesis or solid phase synthesis as described, for example, in Chapter 9 of Atherton and Shephard (supra) and in Roberge et al. (1995, Science 269: 202).

### 4. Nucleic Acid Constructs

In accordance with the present invention nucleic acid constructs are contemplated for inhibiting expression of SUS2 or reducing the level or activity of SUS2 in a sucrose-accumulating crop plant in order to increase the concentration or yield of sucrose or sucrose derivatives in a sucrose-accumulating plant, plant part or plant organ.

These constructs usually comprise in operable connection: (1) a promoter that is operable in a cell of the sucrose-accumulating crop plant (e.g., a plant stem cell); and (2) a nucleic acid sequence encoding an expression product that inhibits expression of a SUS2 nucleic acid molecule as described for example in Section 2, or reduces the level or activity a SUS2 polypeptide as broadly described for example in Section 3.

4.1 Promoters

Any promoter that is operable in cells of a sucrose-accumulating plant, plant part or plant organ is contemplated in the present invention. In some embodiments, promoters useable with the present invention can include those that drive expression of a nucleotide sequence constitutively, those that drive expression when induced, and those that drive expression in a tissue- or developmentally-specific manner.

The promoter may be endogenous to the plant. Alternatively, a heterologous promoter may be employed. For example, a promoter can be heterologous when it is operably linked to a polynucleotide from a species different from the species from which the polynucleotide was derived. Alternatively, a promoter can be heterologous to a selected nucleotide sequence if the promoter is from the same/analogous species from which the polynucleotide is derived, but one or both (i.e., promoter and/or polynucleotide) are modified from their original form and/or genomic locus, or the promoter is not the native promoter for the operably linked polynucleotide.

The choice of promoters useable with the present invention can be made among many different types of promoters. This choice generally depends upon several factors, including, but not limited to, cell- or tissue-specific expression, desired expression level, efficiency, inducibility and/or selectability. For example, where expression in a specific tissue or organ is desired in addition to inducibility, a tissue-specific promoter can be used (e.g., a plant stem cell-specific or -preferential promoter). In contrast, where expression in response to a stimulus is desired, a promoter inducible by that stimulus can be used. Where continuous expression is desired throughout the cells of a plant, a constitutive promoter can be chosen.

Non-limiting examples of constitutive promoters include cestrum virus promoter (cmp) (U.S. Pat. No. 7,166,770), the rice actin 1 promoter (Wang et al. (1992) Mol. Cell. Biol. 12:3399-3406; as well as U.S. Pat. No. 5,641,876), CaMV 35S promoter (Odell et al. (1985) Nature 313:810-812), CaMV 19S promoter (Lawton et al. (1987) Plant Mol. Biol. 9:315-324), nos promoter (Ebert et al. (1987) Proc. Natl. Acad. Sci USA 84:5745-5749), Adh promoter (Walker et al. (1987) Proc. Natl. Acad. Sci. USA 84:6624-6629), sucrose synthase promoter (Yang & Russell (1990) Proc. Natl. Acad. Sci. USA 87:4144-4148), and the ubiquitin promoter.

Illustrative examples of tissue-specific promoters include those encoding the seed storage proteins (such as β-conglycinin, cruciferin, napin and phaseolin), zein or oil body proteins (such as oleosin), or proteins involved in fatty acid biosynthesis (including acyl carrier protein, stearoyl-ACP desaturase and fatty acid desaturases (fad 2-1)), and other nucleic acids expressed during embryo development (such as Bce4, see, e.g., Kridl et al. (1991) Seed Sci. Res. 1:209-219; as well as EP Patent No. 255378). Thus, the promoters associated with these tissue-specific nucleic acids can be used in the present invention. Additional examples of tissue-specific promoters include, but are not limited to, the root-specific promoters RCc3 (Jeong et al. Plant Physiol. 153:185-197 (2010)) and RB7 (U.S. Pat. No. 5,459,252), the lectin promoter (Lindstrom et al. (1990) Der. Genet. 11:160-167; and Vodkin (1983) Prog. Clin. Biol. Res. 138:87-98), corn alcohol dehydrogenase 1 promoter (Dennis et al. (1984) Nucleic Acids Res. 12:3983-4000), S-adenosyl-L-methionine synthetase (SAMS) (Vander Mijnsbrugge et al. (1996) Plant and Cell Physiology, 37(8):1108-1115), corn light harvesting complex promoter (Bansal et al. (1992) Proc. Natl. Acad. Sci. USA 89:3654-3658), corn heat shock protein promoter (O'Dell et al. (1985) EMBO J. 5:451-458; and Rochester et al. (1986) EMBO J. 5:451-458), pea small subunit RuBP carboxylase promoter (Cashmore, “Nuclear genes encoding the small subunit of ribulose-1,5-bisphosphate carboxylase” 29-39 In: Genetic Engineering of Plants (Hollaender ed., Plenum Press 1983; and Poulsen et al. (1986) Mol. Gen. Genet. 205:193-200), Ti plasmid mannopine synthase promoter (Langridge et al. (1989) Proc. Natl. Acad. Sci. USA 86:3219-3223), Ti plasmid nopaline synthase promoter (Langridge et al. (1989), supra), petunia chalcone isomerase promoter (van Tunen et al. (1988) EMBO J. 7:1257-1263), bean glycine rich protein 1 promoter (Keller et al. (1989) Genes Dev. 3:1639-1646), truncated CaMV 35S promoter (O'Dell et al. (1985) Nature 313:810-812), potato patatin promoter (Wenzler et al. (1989) Plant Mol. Biol. 13:347-354), root cell promoter (Yamamoto et al. (1990) Nucleic Acids Res. 18:7449), maize zein promoter (Kriz et al. (1987) Mol. Gen. Genet. 207:90-98; Langridge et al. (1983) Cell 34:1015-1022; Reina et al. (1990) Nucleic Acids Res. 18:6425; Reina et al. (1990) Nucleic Acids Res. 18:7449; and Wandelt et al. (1989) Nucleic Acids Res. 17:2354), globulin-1 promoter (Belanger et al. (1991) Genetics 129:863-872), α-tubulin cab promoter (Sullivan et al. (1989) Mol. Gen. Genet. 215:431-440), PEPCase promoter (Hudspeth & Grula (1989) Plant Mol. Biol. 12:579-589), R gene complex-associated promoters (Chandler et al. (1989) Plant Cell 1:1175-1183), and chalcone synthase promoters (Franken et al. (1991) EMBO J. 10:2605-2612). Particularly useful for seed-specific expression is the pea vicilin promoter (Czako et al. (1992) Mol. Gen. Genet. 235:33-40; as well as U.S. Pat. No. 5,625,136). Other useful promoters for expression in mature leaves are those that are switched on at the onset of senescence, such as the SAG promoter from Arabidopsis (Gan et al. (1995) Science 270:1986-1988).

In specific embodiments, the promoter that is used is one that is specifically or preferentially operable in a plant stem cell. Non-limiting examples of plant stem cell-specific or plant stem cell-preferential promoters include: A1 promoter from sugarcane (Mudge et al. (2009) Planta 229:549-558); ScLSG1 promoter from sugarcane (Moyle et al., Theoretical and Applied Genetics, in press; Plant Molecular Biology, in press); ScLSG4 promoter from sugarcane (Moyle et al., Theoretical and Applied Genetics, in press; Plant Molecular Biology, in press); ScLSG5 promoter from sugarcane (Moyle et al., Theoretical and Applied Genetics, in press; Plant Molecular Biology, in press); ScLSG6 promoter from sugarcane (Moyle et al., Theoretical and Applied Genetics, in press; Plant Molecular Biology, in press); ScLSG7 promoter from sugarcane (Moyle et al., Theoretical and Applied Genetics, in press; Plant Molecular Biology, in press); ScLSG9 promoter from sugarcane (Moyle et al., Theoretical and Applied Genetics, in press; Plant Molecular Biology, in press); 51 promoter from sugarcane (Potier et al., 2008 Proc S Afr Sug Technol Ass 81: 508-512); 67 promoter from sugarcane (Potier and Birch 2001 Patent Application, WO 01/18221 A1); ProDIR16 from sugarcane (Damaj 2010 Planta 231: 1439-1458); ProOMT from sugarcane (Damaj 2010 Planta 231: 1439-1458); JAS promoter from sugarcane (Damaj et al., 2007 U.S. Pat. No. 7,253,276).

4.2 Expression Products for Inhibiting SUS2 or Protein Products Thereof

The constructs of the present invention also comprise an operably connected nucleic acid sequence encoding an expression product that inhibits expression of a SUS2 nucleic acid molecule, or that reduces the level or activity a SUS2 polypeptide. In some embodiments, the expression product inhibits or abrogates the activity or function of an endogenous SUS2 polypeptide of the plant.

In some embodiments, the expression product inhibits by RNA interference (RNAi) or post-transcriptional gene silencing (PTGS) the expression of an endogenous SUS2 gene. In illustrative examples of this type, the expression product is a RNA molecule (e.g., siRNA, shRNA, miRNA, dsRNA etc.) that comprises a targeting region corresponding to a SUS2 target gene of a sucrose-accumulating crop plant, wherein the SUS2 target gene corresponds for example to a SUS2 nucleic sequence, as described herein, wherein the RNA molecule attenuates or otherwise disrupts the expression of the target gene.

In certain embodiments, the targeting sequence displays at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% identity to a nucleotide sequence of the SUS2 target gene. In other embodiments, the targeting sequence hybridizes to a nucleotide sequence of the target gene under at least low stringency conditions, more suitably under at least medium stringency conditions and even more suitably under high stringency conditions as defined herein.

Suitably, the targeting region has sequence identity with the sense strand or antisense strand of the target gene. In certain embodiments, the RNA molecule is unpolyadenylated, which can lead to efficient reduction in expression of the target gene, as described for example by Waterhouse et al in U.S. Pat. No. 6,423,885.

Typically, the length of the targeting region may vary from about 10 nucleotides (nt) up to a length equaling the length (in nucleotides) of the target gene. Generally, the length of the targeting region is at least 10, 12, 13, 14, 15, 16, 17, 18, 19, 20, 21, 22, 23, 24, 25 nt, usually at least about 50 nt, more usually at least about 100 nt, especially at least about 150 nt, more especially at least about 200 nt, even more especially at least about 500 nt. It is expected that there is no upper limit to the total length of the targeting region, other than the total length of the target gene. However for practical reason (such as e.g., stability of the targeting constructs) it is expected that the length of the targeting region should not exceed 5000 nt, particularly should not exceed 2500 nt and could be limited to about 1000 nt.

The RNA molecule may further comprise one or more other targeting regions (e.g., from about 1 to about 10, or from about 1 to about 4, or from about 1 to about 2 other targeting regions) each of which has sequence identity with a nucleotide sequence of the target gene. Generally, the targeting regions are identical or share at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% sequence identity with each other.

The RNA molecule may further comprise a reverse complement of the targeting region. Typically, in these embodiments, the RNA molecule further comprises a spacer sequence that spaces the targeting region from the reverse complement. The spacer sequence may comprise a sequence of nucleotides of at least about 100-500 nucleotides in length, or alternatively at least about 50-100 nucleotides in length and in a further alternative at least about 10-50 nucleotides in length. Typically, the spacer sequence is a non-coding sequence, which in some instances is an intron. In embodiments in which the spacer sequence is a non-intron spacer sequence, transcription of the nucleic acid sequence will produce an RNA molecule that forms a hairpin or stem-loop structure in which the stem is formed by hybridization of the targeting region to the reverse complement and the loop is formed by the non-intron spacer sequence connecting these ‘inverted repeats’. Alternatively, in embodiments in which the spacer sequence is an intron spacer sequence, the presence of intron/exon splice junction sequences on either side of the intron sequence facilitates the removal of what would otherwise form a loop structure and the resulting RNA will form a double-stranded RNA (dsRNA) molecule, with optional overhanging 3′ sequences at one or both ends. Such a dsRNA transcript is referred to herein as a “perfect hairpin”. The RNA molecules may comprise a single hairpin or multiple hairpins including “bulges” of single-stranded RNA occurring adjacent to regions of double-stranded RNA sequences.

Alternatively, a dsRNA molecule as described above can be conveniently obtained using an additional polynucleotide from which a further RNA molecule is producible, comprising the reverse complement of the targeting region. In this embodiment, the reverse complement of the targeting region hybridizes to the targeting region of the RNA molecule transcribed from the second polynucleotide.

In another example, a dsRNA molecule as described above is prepared using a second polynucleotide that comprises a duplex, wherein one strand of the duplex shares sequence identity with a nucleotide sequence of the target gene and the other shares sequence identity with the complement of that nucleotide sequence. In this embodiment, the duplex is flanked by two promoters, one controlling the transcription of one of the strands, and the other controlling the transcription of the complementary strand. Transcription of both strands produces a pair of RNA molecules, each comprising a region that is complementary to a region of the other, thereby producing a dsRNA molecule that inhibits the expression of the target gene.

In another example, PTGS of the target gene is achieved using the strategy by Glassman et al described in U.S. Patent Application Publication No 2003/0036197. In this strategy, suitable nucleic acid sequences and their reverse complement can be used to alter the expression of any homologous, endogenous target RNA (i.e., comprising a transcript of the target gene) which is in proximity to the suitable nucleic acid sequence and its reverse complement. The suitable nucleic acid sequence and its reverse complement can be either unrelated to any endogenous RNA in the host or can be encoded by any nucleic acid sequence in the genome of the host provided that nucleic acid sequence does not encode any target mRNA or any sequence that is substantially similar to the target RNA. Thus, in some embodiments of the present invention, the RNA molecule further comprises two complementary RNA regions which are unrelated to any endogenous RNA in the host cell and which are in proximity to the targeting region. In other embodiments, the RNA molecule further comprises two complementary RNA regions which are encoded by any nucleic acid sequence in the genome of the host provided that the sequence does not have sequence identity with the nucleotide sequence of the target gene, wherein the regions are in proximity to the targeting region. In the above embodiments, one of the complementary RNA regions can be located upstream of the targeting region and the other downstream of the targeting region. Alternatively, both the complementary regions can be located either upstream or downstream of the targeting region or can be located within the targeting region itself.

In some illustrative examples, the RNA molecule is an antisense molecule that is targeted to a specific region of RNA encoded by the target gene, which is critical for translation. The use of antisense molecules to decrease expression levels of a pre-determined gene is known in the art. Antisense molecules may be designed to correspond to full-length RNA transcribed from the target gene, or to a fragment or portion thereof. This gene silencing effect can be enhanced by transgenically over-producing both sense and antisense RNA of the target gene coding sequence so that a high amount of dsRNA is produced as described for example above (see, for example, Waterhouse et al. (1998) Proc Natl Acad Sci USA 95:13959 13964).

In other embodiments, the expression product that inhibits expression of SUS2 corresponds to an expression product of the endogenous target gene targeted for repression. In many cases, this “co-suppression” results in the complete repression of the native target gene as well as the transgene.

In other embodiments, the encoded expression product is an antibody that is immuno-interactive with an endogenous SUS2 polypeptide. Exemplary antibodies for use in the practice of the present invention include monoclonal antibodies, Fv, Fab, Fab′ and F(ab′)2 immunoglobulin fragments, as well as synthetic antibodies such as but not limited to single domain antibodies (DABs), synthetic stabilized Fv fragments, e.g., single chain Fv fragments (scFv), disulfide stabilized Fv fragments (dsFv), single variable region domains (dAbs) minibodies, combibodies and multivalent antibodies such as diabodies and multi-scFv or engineered human equivalents. Techniques for preparing and using various antibody-based constructs and fragments are well known in the art. Means for preparing and characterizing antibodies are also well known in the art. In illustrative examples, antibodies can be made by conventional immunization (e.g., polyclonal sera and hybridomas) with isolated, purified or recombinant peptides or proteins corresponding to at least a portion of an endogenous polypeptide, or as recombinant fragments corresponding to at least a portion of an endogenous polypeptide, usually expressed in Escherichia coli, after selection from phage display or ribosome display libraries (e.g., available from Cambridge Antibody Technology, Biolnvent, Affitech and Biosite). Knowledge of the antigen-binding regions (e.g., complementarity-determining regions) of such antibodies can be used to prepare synthetic antibodies as described for example above.

4.3 Other Construct Elements

In addition to the operably linked promoter and nucleic acid sequence encoding an expression product that inhibits expression of SUS2 or reduces the level or activity of SUS2, the constructs of the present invention, which are suitably expression constructs, can also include other regulatory sequences. As used herein, “regulatory sequences” means nucleotide sequences located upstream (5′ non-coding sequences), within or downstream (3′ non-coding sequences) of a coding sequence, and which influence the transcription, RNA processing or stability, or translation of the associated coding sequence. Regulatory sequences include, but are not limited to, enhancers, introns, translation leader sequences and polyadenylation signal sequences.

A number of non-translated leader sequences derived from viruses are known to enhance gene expression. Specifically, leader sequences from Tobacco Mosaic Virus (TMV, the “Q-sequence”), Maize Chlorotic Mottle Virus (MCMV) and Alfalfa Mosaic Virus (AMV) have been shown to be effective in enhancing expression (Gallie et al. (1987) Nucleic Acids Res. 15:8693-8711; and Skuzeski et al. (1990) Plant Mol. Biol. 15:65-79). Other leader sequences known in the art include, but are not limited to, picornavirus leaders such as an encephalomyocarditis (EMCV) 5′ noncoding region leader (Elroy-Stein et al. (1989) Proc. Natl. Acad. Sci. USA 86:6126-6130); potyvirus leaders such as a Tobacco Etch Virus (TEV) leader (Allison et al. (1986) Virology 154:9-20); Maize Dwarf Mosaic Virus (MDMV) leader (Allison et al. (1986), supra); human immunoglobulin heavy-chain binding protein (BiP) leader (Macejak & Samow (1991) Nature 353:90-94); untranslated leader from the coat protein mRNA of AMV (AMV RNA 4; Jobling & Gehrke (1987) Nature 325:622-625); tobacco mosaic TMV leader (Gallie et al. (1989) Molecular Biology of RNA 237-256); and MCMV leader (Lommel et al. (1991) Virology 81:382-385). See also, Della-Cioppa et al. (1987) Plant Physiol. 84:965-968. In some embodiments, translational enhancers are employed such as the overdrive-sequence containing the 5′-untranslated leader sequence from tobacco mosaic virus enhancing the polypeptide per RNA ratio (Gallie et al. (1987) Nucleic Acids Research 15:8693-8711).

An expression construct also can optionally include a transcriptional and/or translational termination region (i.e., termination region) that is functional in plants. A variety of transcriptional terminators are available for use in expression constructs and are responsible for the termination of transcription beyond the heterologous nucleotide sequence of interest and correct mRNA polyadenylation. The termination region may be native to the transcriptional initiation region, may be native to the operably linked nucleotide sequence of interest, may be native to the plant host, or may be derived from another source (i.e., foreign or heterologous to the promoter, the nucleotide sequence of interest, the plant host, or any combination thereof). Appropriate transcriptional terminators include, but are not limited to, the CAMV 35S terminator, the tml terminator, the nopaline synthase terminator and the pea rbcs E9 terminator. These can be used in both monocotyledons and dicotyledons. In addition, a coding sequence's native transcription terminator can be used. A signal sequence can be operably linked to a nucleic acid molecule of the present invention to direct the nucleic acid molecule into a cellular compartment. In this manner, the expression construct will comprise a nucleic acid molecule of the present invention operably linked to a nucleotide sequence for the signal sequence. The signal sequence may be operably linked at the N- or C-terminus of the nucleic acid molecule. Exemplary polyadenylation signals can be those originating from Agrobacterium tumefaciens t-DNA such as the gene known as octopine synthase of the Ti-plasmid pTiACH5 (Gielen et al. (1984) EMBO J. 3:835) or functional equivalents thereof, but also all other terminators functionally active in plants are suitable.

The expression construct also can include a nucleotide sequence for a selectable marker, which can be used to select a transformed plant, plant part and/or plant cell. As used herein, “selectable marker” means a nucleotide sequence that when expressed imparts a distinct phenotype to the plant, plant part and/or plant cell expressing the marker and thus allows such transformed plants, plant parts and/or plant cells to be distinguished from those that do not have the marker. Such a nucleotide sequence may encode either a selectable or screenable marker, depending on whether the marker confers a trait that can be selected for by chemical means, such as by using a selective agent (e.g., an antibiotic, herbicide, or the like), or on whether the marker is simply a trait that one can identify through observation or testing, such as by screening (e.g., the R-locus trait). Of course, many examples of suitable selectable markers are known in the art and can be used in the expression constructs described herein.

Examples of selectable markers include, but are not limited to, a nucleotide sequence encoding neo or nptII, which confers resistance to kanamycin, G418, and the like (Potrykus et al. (1985) Mol. Gen. Genet. 199:183-188); a nucleotide sequence encoding bar, which confers resistance to phosphinothricin; a nucleotide sequence encoding an altered 5-enolpyruvylshikimate-3-phosphate (EPSP) synthase, which confers resistance to glyphosate (Hinchee et al. (1988) Biotech. 6:915-922); a nucleotide sequence encoding a nitrilase such as bxn from Klebsiella ozaenae that confers resistance to bromoxynil (Stalker et al. (1988) Science 242:419-423); a nucleotide sequence encoding an altered acetolactate synthase (ALS) that confers resistance to imidazolinone, sulfonylurea or other ALS-inhibiting chemicals (EP Patent Application No. 154204); a nucleotide sequence encoding a methotrexate-resistant dihydrofolate reductase (DHFR) (Thillet et al. (1988) J. Biol. Chem. 263:12500-12508); a nucleotide sequence encoding a dalapon dehalogenase that confers resistance to dalapon; a nucleotide sequence encoding a mannose-6-phosphate isomerase (also referred to as phosphomannose isomerase (PMI)) that confers an ability to metabolize mannose (U.S. Pat. Nos. 5,767,378 and 5,994,629); a nucleotide sequence encoding an altered anthranilate synthase that confers resistance to 5-methyl tryptophan; and/or a nucleotide sequence encoding hph that confers resistance to hygromycin. One of skill in the art is capable of choosing a suitable selectable marker for use in an expression construct of this invention.

Additional selectable markers include, but are not limited to, a nucleotide sequence encoding β-glucuronidase or uidA (GUS) that encodes an enzyme for which various chromogenic substrates are known; an R-locus nucleotide sequence that encodes a product that regulates the production of anthocyanin pigments (red color) in plant tissues (Dellaporta et al., “Molecular cloning of the maize R-nj allele by transposon-tagging with Ac” 263-282 In: Chromosome Structure and Function: Impact of New Concepts, 18th Stadler Genetics Symposium (Gustafson & Appels eds., Plenum Press 1988)); a nucleotide sequence encoding β-lactamase, an enzyme for which various chromogenic substrates are known (e.g., PADAC, a chromogenic cephalosporin) (Sutcliffe (1978) Proc. Natl. Acad. Sci. USA 75:3737-3741); a nucleotide sequence encoding xylE that encodes a catechol dioxygenase (Zukowsky et al. (1983) Proc. Natl. Acad. Sci. USA 80:1101-1105); a nucleotide sequence encoding tyrosinase, an enzyme capable of oxidizing tyrosine to DOPA and dopaquinone, which in turn condenses to form melanin (Katz et al. (1983) J. Gen. Microbiol. 129:2703-2714); a nucleotide sequence encoding β-galactosidase, an enzyme for which there are chromogenic substrates; a nucleotide sequence encoding luciferase (lux) that allows for bioluminescence detection (Ow et al. (1986) Science 234:856-859); a nucleotide sequence encoding aequorin which may be employed in calcium-sensitive bioluminescence detection (Prasher et al. (1985) Biochem. Biophys. Res. Comm. 126:1259-1268); or a nucleotide sequence encoding green fluorescent protein (Niedz et al. (1995) Plant Cell Reports 14:403-406). One of skill in the art is capable of choosing a suitable selectable marker for use in an expression construct of this invention.

An expression construct of the present invention also can include nucleotide sequences that encode other desired traits. Such nucleotide sequences can be stacked with any combination of nucleotide sequences to create plants, plant parts or plant cells having the desired phenotype. Stacked combinations can be created by any method including, but not limited to, cross breeding plants by any conventional methodology, or by genetic transformation. If stacked by genetically transforming the plants, the nucleotide sequences of interest can be combined at any time and in any order. For example, a transgenic plant comprising one or more desired traits can be used as the target to introduce further traits by subsequent transformation. The additional nucleotide sequences can be introduced simultaneously in a co-transformation protocol with a nucleotide sequence, nucleic acid molecule, nucleic acid construct, and/or composition of this invention, provided by any combination of expression constructs. For example, if two nucleotide sequences will be introduced, they can be incorporated in separate cassettes (trans) or can be incorporated on the same cassette (cis). Expression of the nucleotide sequences can be driven by the same promoter or by different promoters. It is further recognized that nucleotide sequences can be stacked at a desired genomic location using a site-specific recombination system. See, e.g., Intl Patent Application Publication Nos. WO 99/25821; WO 99/25854; WO 99/25840; WO 99/25855 and WO 99/25853.

In addition to the nucleic acid encoding an expression product that inhibits expression of SUS2 or reduces the level or activity of SUS2, the expression construct can include a coding sequence for one or more polypeptides for agronomic traits that primarily are of benefit to a seed company, grower or grain processor. A polypeptide of interest can be any polypeptide encoded by a nucleotide sequence of interest. Non-limiting examples of polypeptides of interest that are suitable for production in plants include those resulting in agronomically important traits such as herbicide resistance (also sometimes referred to as “herbicide tolerance”), virus resistance, bacterial pathogen resistance, insect resistance, nematode resistance, and/or fungal resistance. See, e.g., U.S. Pat. Nos. 5,569,823; 5,304,730; 5,495,071; 6,329,504; and 6,337,431. The polypeptide also can be one that increases plant vigor or yield (including traits that allow a plant to grow at different temperatures, soil conditions and levels of sunlight and precipitation), or one that allows identification of a plant exhibiting a trait of interest (e.g., a selectable marker, seed coat color, etc.). Various polypeptides of interest, as well as methods for introducing these polypeptides into a plant, are described, for example, in U.S. Pat. Nos. 6,084,155; 6,329,504 and 6,337,431; as well as U.S. Patent Publication No. 2001/0016956. See also, on the World Wide Web at lifesci.sussex.ac.uk/home/Neil_Crickmore/Bt/. Nucleotide sequences conferring resistance/tolerance to an herbicide that inhibits the growing point or meristem, such as an imidazalinone or a sulfonylurea can also be suitable in some embodiments of the invention. Exemplary nucleotide sequences in this category code for mutant ALS and AHAS enzymes as described, e.g., in U.S. Pat. Nos. 5,767,366 and 5,928,937. U.S. Pat. Nos. 4,761,373 and 5,013,659 are directed to plants resistant to various imidazalinone or sulfonamide herbicides. U.S. Pat. No. 4,975,374 relates to plant cells and plants containing a nucleic acid encoding a mutant glutamine synthetase (GS) resistant to inhibition by herbicides that are known to inhibit GS, e.g., phosphinothricin and methionine sulfoximine. U.S. Pat. No. 5,162,602 discloses plants resistant to inhibition by cyclohexanedione and aryloxyphenoxypropanoic acid herbicides. The resistance is conferred by an altered acetyl coenzyme A carboxylase (ACCase).

### 5. SUS2 Inactivation

Alternatively, SUS2 expression or the level or activity of SUS2 may be reduced or inhibited by inactivating SUS2 in the genome of a plant. In some embodiments, SUS2 is modified by mutagenesis. Genetic mutations (e.g., loss-of-function mutations) can be introduced within regenerable plant cells or tissues using one or more mutagenic agents. Suitable mutagenic agents include, for example, ethyl methane sulfonate (EMS), N-nitroso-N-ethylurea (ENU), methyl N-nitrosoguanidine (MNNG), ethidium bromide (EtBr), diepoxybutane, ionizing radiation, ionizing radiation, x-rays, ultra violet rays, gamma rays, fast neutrons and other mutagens known in the art. Suitable types of mutations include, for example, insertions or deletions of nucleotides, and transitions or transversions in a SUS2 gene.

In some embodiments, TILLING (Targeted Induced Local Lesions In Genomes) is used to produce plants having a modified endogenous nucleic acid. TILLING combines high-density mutagenesis with high-throughput screening methods. See, for example, McCallum et al. (2000, Nat Biotechnol 18:455-457); reviewed by Stemple (2004, Nat Rev Genet. 5(2):145-50).

Reference also may be made to the mutagenesis methods disclosed by Sikora et al. (2011, International Journal of Plant Genomics Volume 2011, Article ID 314829, doi:10.1155/2011/314829), Nura et al. (2011, Continental J. Biological Sciences 4(2): 22-27), Conradie, T T. (Masters Thesis, “Genetic engineering of sugarcane for increased sucrose and consumer acceptance.” Stellebosch University, 2011) and Navarro et al. (2006 J Biol Chem., 281, 13268-13274).

In some embodiments, at least one plant cell is treated with a chemical mutagenizing agent (e.g., EMS, ENU, MNNG, EtBr, diepoxybutane) under conditions effective to yield at least one mutant plant cell containing an inactive SUS2 gene.

In other embodiments, at least one plant cell is subjected to a radiation source (e.g., ionizing radiation, x-rays, ultra violet rays, gamma rays, fast neutrons etc.) under conditions effective to yield at least one mutant plant cell containing an inactive SUS2 gene.

In still other embodiments, at least one plant cell is treated by inserting an inactivating nucleic acid molecule into the SUS2 gene encoding a functional SUS2 protein or its promoter under conditions effective to inactivate the gene. In illustrative examples of this type, the inactivating nucleic acid is a transposable element (e.g., an Activator (Ac) transposon, a Dissociator (Ds) transposon, a Mutator (Mu) transposon etc.).

In other embodiments, at least one plant cell is subjected to Agrobacterium transformation under conditions effective to insert an Agrobacterium T-DNA sequence into the SUS2 gene, thereby inactivating the gene.

In yet other embodiments, at least one plant cell is subjected to site-directed mutagenesis of the SUS2 gene or its promoter under conditions effective to yield at least one mutant plant cell containing an inactive SUS2 gene. In illustrative examples of this type, the mutagenesis comprises homologous recombination of the SUS2 gene or its promoter (e.g., targeted deletion of a portion of the SUS2 gene sequence or its promoter or targeted insertion of a nucleic acid sequence into the SUS2 gene or its promoter).

### 6. Transgenic Plants, Plant Parts, Plant Organs and Plant Cells

The present invention further encompasses plant cells, plant parts, plant organs and plants in accordance with the embodiments of this invention. Thus, in some embodiments, the present invention provides a transformed plant cell, plant part, plant organ and/or plant comprising a nucleic acid molecule, a nucleic acid construct, a nucleotide sequence, a promoter, and/or a composition of this invention. Representative plants include, for example, angiosperms (monocots and dicots), gymnosperms, bryophytes, ferns and/or fern allies.

In some embodiments, the plants are selected from monocotyledonous plants. Non-limiting examples of monocot plants include sugar cane, corn, barley, rye, oats, wheat, rice, flax, millet, sorghum, grasses (e.g., switch grass, giant reed, turf grasses etc.), banana, onion, asparagus, lily, coconut, and the like. In some embodiments, the monocot plants of the invention include plants of the genus Saccharum (i.e., sugar cane, energy cane) and hybrids thereof, including hybrids between plants of the genus Saccharum and those of related genera, such as Miscanthus, Erianthus, Sorghum and others. As used herein, “sugar cane” and “Saccharum spp.” mean any of six to thirty-seven species (depending on taxonomic interpretation) of tall perennial grasses of the genus Saccharum. In particular, the plant can be Saccharum aegyptiacum, Saccharum esculentum, Saccharum arenicol, Saccharum arundinaceum, Saccharum barberi, Saccharum bengalense, Saccharum biflorum, Saccharum chinense, Saccharum ciliare, Saccharum cylindricum, Saccharum edule, Saccharum elephantinurn, Saccharum exaltaturn, Saccharum fallax, Saccharum fallax, Saccharum floridulum, Saccharum giganteum, Saccharum hybridum, Saccharum japonicum, Saccharum koenigii, Saccharum laguroides, Saccharum munja, Saccharum narenga, Saccharum officinale, Saccharum officinarum, Saccharum paniceum, Saccharum pophyrocoma, Saccharum purpuratum, Saccharum ravennae, Saccharum robustum, Saccharum roseum, Saccharum sanguineum, Saccharum sara, Saccharum sinense, Saccharum spontaneum, Saccharum tinctorium, Saccharum versicolor, Saccharum violaceum, Saccharum violaceum, and any of the interspecific hybrids and commercial varieties thereof.

Further non-limiting examples of plants of the present invention include soybean, beans in general, Brassica spp., clover, cocoa, coffee, cotton, peanut, rape/canola, safflower, sugar beet, sunflower, sweet potato, tea, vegetables including but not limited to broccoli, brussel sprouts, cabbage, carrot, cassava, cauliflower, cucurbits, lentils, lettuce, pea, peppers, potato, radish and tomato, fruits including, but not limited to, apples, pears, peaches, apricots and citrus, avocado, pineapple and walnuts, and any combination thereof.

In some embodiments, the plants are selected from energy crops, representative examples of which include:

Beta vulgaris L., Beta vulgaris subsp. adanensis (Pamukç.), Beta vulgaris var. altissima Döll, Beta vulgaris subsp. asiatica Krassochkin ex Burenin Synonym, Beta vulgaris var. asiatica Burenin Synonym, Beta vulgaris var. atriplicifolia (Rouy) Krassochkin Synonym, Beta vulgaris var. aurantia Burenin Synonym, Beta vulgaris subsp. cicla (L.) Schübl. & G.Martens Synonym, Beta vulgaris var. cicla L. Synonym, Beta vulgaris subsp. cicla (L.) W.D.J. Koch Synonym, Beta vulgaris var. coniciformis Burenin Synonym, Beta vulgaris var. foliosa (Asch. & Schweinf.) Aellen Synonym, Beta vulgaris subsp. foliosa Asch. & Schweinf. Synonym, Beta vulgaris var. glabra (Delile) Aellen Synonym, Beta vulgaris var. grisea Aellen Synonym, Beta vulgaris subsp. lomatogonoides Aellen Synonym, Beta vulgaris var. macrocarpa (Guss.) Moq. Synonym, Beta vulgaris subsp. macrocarpa (Guss.) Thell. Synonym, Beta vulgaris var. marcosii O. Bolós & Vigo Synonym, Beta vulgaris var. maritima (L.) Alef. Synonym, Beta vulgaris subsp. maritima (L.) Arcang. Synonym, Beta vulgaris subsp. maritima (L.) Thell. Synonym, Beta vulgaris var. maritima (L.) Moq. Synonym, Beta vulgaris var. mediasiatica Burenin Synonym, Beta vulgaris var. orientalis (Roth) Moq. Synonym, Beta vulgaris subsp. orientalis (Roth) Aellen Synonym, Beta vulgaris var. ovaliformis Burenin Synonym, Beta vulgaris subsp. patula (Aiton) Ford-Lloyd & J.T. Williams Synonym, Beta vulgaris var. perennis L. Synonym, Beta vulgaris var. pilosa (Delile) Moq. Synonym, Beta vulgaris subsp. provulgaris Ford-Lloyd & J.T. Williams Synonym, Beta vulgaris var. rosea Moq. Synonym, Beta vulgaris var. rubidus Burenin Synonym, Beta vulgaris var. rubra L. Synonym, Beta vulgaris var. rubrifolia Krassochkin ex Burenin Synonym, Beta vulgaris var. saccharifera Alef. Synonym, Beta vulgaris var. trojana (Pamukç), Beta vulgaris var. virescens Burenin Synonym, Beta vulgaris var. viridifolia Krassochkin ex Burenin Synonym, Beta vulgaris var. vulgaris, Beta vulgaris subsp. vulgaris

Saccharum (e.g., as described above including S. ravennae and S. sponteneum);

Sorghum (e.g., Sorghum abyssinicum, S. aethiopicum, S. album, S. andropogon, S. ankolib, S. annuum, S. anomalum, S. arctatum, S. arduini, S. arenarium, S. argenteum, S. arunidinaceum, S. arvense, S. asperum, S. aterrimum, S. australiense, S. avenaceum, S. balansae, S. bantuorum, S. barbatum, S. basiplicatum, S. basutorum, S. bicome, S. bipennatum, S. bourgaei, S. brachystachyum, S. bracteatum, S. brevicallosum, S. brevicarinatum, S. brevifolium, S. burmahicum, S. cabanisii, S. caffrorum, S. campanum, S. campestre, S. camporum, S. caudatum, S. canescens, S. capense, S. capillare, S. carinatum, S. castaneum, S. caucasicum, S. caudatum, S. centroplicatum, S. cernum, S. cemuum, S. chinense, S. chinese, S. cirratum, S. commune, S. compactum, S. condensatum, S. consanguineum, S. conspicuum, S. contortum, S. controversum, S. coriaceum, S. crupina, S. cubanicus, S. cubense, S. deccanense, S. decolor, S. decolorans, S. dimidiatum, S. dochna, S. dora, S. dubium, S. dulcicaule, S. durra, S. elegans, S. elliotii, S. elliottii, S. elongatum, S. eplicatum, S. exaratum, S. exsertum, S. fastigiatum, S. fauriei, S. flavescens, S. flavum, S. friesii, S. fulvum, S. fuscum, S. gambicum, S. giganteum, S. glabrescens, S. glaucescens, S. glaziovii, S. glomeratum, S. glycychylum, S. gracile, S. gracilipes, S. grandes, S. guineence, S. guineence, S. guinense, S. halapense, S. halenpensis, S. halepensis, S. hallii, S. hewisonii, S. hirse, S. hirtiflorum, S. hirtifolium, S. hirtum, S. hybrid, S. incompletum, S. japonicum, S. junghuhnii, S. lanceolatum, S. laterale, S. laxum, S. leidadum, S. leptocladum, S. leptos, S. leucostachyum, S. liebmanni, S. liebmannii, S. lithophilum, S. longiberbe, S. macrochaeta, S. malacostachyum, S. margaritiferum, S. medioplicatum, S. mekongense, S. melaleucum, S. melanocarpum, S. mellitum, S. membranaceum, S. micratherum, S. miliaceum, S. miliiforme, S. minarum, S. mixture, S. mjoebergii, S. muticum, S. myosurus, S. nankinense, S. negrosense, S. nervosum, S. nigericum, S. nigricans, S. nigrum, S. niloticum, S. nitens, S. notabile, S. nubicum, S. nutans, S. orysoidum, S. pallidum, S. panicoides, S. papyrascens, S. parviflorum, S. pauciflorum, S. piptatherum, S. platyphyllum, S. pogonostachyum, S. pohlianum, S. provinciale, S. pugionifolium, S. purpureo-sericeum, S. pyramidale, S. quartinianum, S. repens, S. riedelii, S. rigidifolium, S. rigidum, S. rollii, S. roxburghii, S. rubens, S. rufum, S. ruprechtii, S. saccharatum, S. saccharoides, S. salzmanni, S. sativum, S. scabriflorum, S. schimperi, S. schlumbergeri, S. schottii, S. schreberi, S. scoparium, S. secundum, S. semiberbe, S. serrature, S. setifolium, S. simulans, S. somaliense, S. sorghum, S. spathiflorum, S. splendidum, S. spontaneum, S. stapfii, S. striatum, S. subglabrescens, S. sudanense, S. tataricum, S. technicum, S. technicus, S. tenerum, S. tematurn, S. thonizzi, S. trichocladum, S. trichopus, S. tropicum, S. truchmenorum, S. usambarense, S. usorum, S. verticillatum, S. verticilliflorum, S. vestitum, S. villosum, S. virgatum, S. virginicum, S. vogelianum, S. vulgare, S. wrightii, S. zeae, S. zollingeri Hybrid: S. × almum, S. × almum Parodi, S. bicolor × sudanense, S. × derzhavinii, S. × drummondii, S. × randolphianum);

wheat (e.g., Triticum abyssinicum, T. accessorium, T. acutum, T. aegilapoides, T. aegilopoides, T. aegilops, T. aesticum, T. aestivum, T. aethiopicum, T. affine, T. afghanicum, T. agropyrotriticum, T. alatum, T. album, T. algeriense, T. alpestre, T. alpinum, T. amyleum, T. amylosum, T. angustifolium, T. angustum, T. antiquorum, T. apiculatum, T. aragonense, T. aralense, T. araraticum, T. arenarium, T. arenicolum, T. arias, T. aristatum, T. arktasianum, T. armeniacum, T. arras, T. arundinaceum, T. arvense, T. asiaticum, T. asperrimum, T. asperum, T. athericum, T. atratum, T. attenuatum, T. aucheri, T. baeoticum, T. barbinode, T. barbulatum, T. barrelieri, T. batalini, T. bauhini, T. benghalense, T. bicorne, T. bifaria, T. biflorum, T. biunciale, T. bonaepartis, T. boreale, T. borisovii, T. brachystachyon, T. brachystachyum, T. breviaristatum, T. brevisetum, T. brizoides, T. bromoides, T. brownei, T. bucharicum, T. bulbosum, T. bungeanum, T. buonapartis, T. burnaschewi, T. caeruleum, T. caesium, T. caespitosum, T. campestre, T. candissimum, T. caninum, T. capense, T. carthlicum, T. caucasicum, T. caudatum, T. cereale, T. cerulescens, T. cevallos, T. chinense, T. cienfuegos, T. ciliare, T. ciliatum, T. cinereum, T. clavatum, T. coarctatum, T. cochleare, T. comosum, T. compactum, T. compositum, T. compressum, T. condensatum, T. crassum, T. cretaceum, T. creticum, T. crinitum, T. cristatum, T. curvifolium, T. cylindricum, T. cynosuroides, T. czernjaevi, T. dasyanthum, T. dasyphyllum, T. dasystachys, T. dasystachyum, T. densiflorum, T. densiusculum, T. desertorum, T. dichasians, T. dicoccoides, T. dicoccon, T. dicoccum, T. distachyon, T. distans, T. distertum, T. distichum, T. divaricatum, T. divergens, T. diversifolium, T. donianum, T. dumetorum, T. duplicatum, T. duriusculum, T. duromedium, T. durum, T. duvalii, T. elegans, T. elongatum, T. elymogenes, T. elymoides, T. emarginatum, T. erebuni, T. erinaceum, T. farctum, T. farrum, T. fastuosum, T. festuca, T. festucoides, T. fibrosum, T. filiforme, T. firmum, T. flabellatum, T. flexura, T. forskalei, T. fragile, T. freycenetii, T. fuegianum, T. fungicidum, T. gaertnerianum, T. geminatum, T. geniculatum, T. genuense, T. giganteum, T. glaucescens, T. glaucum, T. gmelini, T. gracile, T. halleri, T. hamosum, T. hebestachyum, T. heldreichii, T. hemipoa, T. hieminflatum, T. hirsutum, T. hispanicum, T. hordeaceum, T. hordeiforme, T. hornemanni, T. horstianum, T. hosteanum, T. hybemum, T. ichyostachyum, T. imbricatum, T. immaturatum, T. infestum, T. inflatum, T. intermedium, T. ispahanicum, T. jakubzineri, T. juncellum, T. junceum, T. juvenale, T. kiharae, T. kingianum, T. kirgianum, T. koeleri, T. kosanini, T. kotschyanum, T. kotschyi, T. labile, T. lachenalii, T. laevissimum, T. lasianthum, T. latiglume, T. latronum, T. laxiusculum, T. laxum, T. leersianum, T. ligusticum, T. linnaeanum, T. litorale, T. litoreum, T. littoreum, T. loliaceum, T. lolioides, T. longearistatum, T. longisemineum, T. longissimum, T. lorentii, T. lutinflatum, T. luzonense, T. macha, T. macrochaetum, T. macrostachyum, T. macrourum, T. magellanicum, T. maritimum, T. markgrafii, T. martius, T. maturatum, T. maurorum, T. maximum, T. mexicanum, T. miguschovae, T. missuricum, T. molle, T. monococcum, T. monostachyum, T. multiflorum, T. murale, T. muricatum, T. nardus, T. neglectum, T. nigricans, T. nodosum, T. nubigenum, T. obtusatum, T. obtusiflorum, T. obtusifolium, T. obtusiusculum, T. olgae, T. orientate, T. ovatum, T. palaeo-colchicum, T. palmovae, T. panarmitanum, T. paradoxum, T. patens, T. patulum, T. pauciflorum, T. pectinatum, T. pectiniforme, T. percivalianum, T. peregrinum, T. persicum, T. peruvianum, T. petraeum, T. petropavlovskyi, T. phaenicoides, T. phoenicoides, T. pilosum, T. pinnatum, T. planum, T. platystachyum, T. poa, T. poliens, T. polonicum, T. poltawense:. m polystachyum, i. nticum, T. pouzolzii, T. proliferum, T. prostratum, T. pruinosum, T. pseudo-agropyrum, T. pseudocaninum, T. puberulum, T. pubescens, T. pubiflorum, T. pulverulentum, T. pumilum, T. pungens, T. pycnanthum, T. pyramidale, T. quadratum, T. ramificum, T. ramosum, T. rarum, T. recognitum, T. rectum, T. repens, T. reptans, T. requlenii, T. richardsonii, T. rigidum, T. rodeti, T. roegnerii, T. rossicum, T. rottboellia, T. rouxii, T. rufescens, T. rufinflatum, T. rupestre, T. sabulosum, T. salinum, T. salsuginosum, T. sanctum, T. sardinicum, T. sartarii, T. sativum, T. savignionii, T. savignonii, T. scaberrimum, T. scabrum, T. schimperi, T. schrenkianum, T. scirpeum, T. secale, T. secalinum, T. secundum, T. segetale, T. semicostatum, T. sepium, T. sibiricum, T. siculum, T. siliginum, T. silvestre, T. simplex, T. sinaicum, T. sinskajae, T. solandri, T. sparsum, T. spelta, T. speltaeforme, T. speltoides, T. sphaerococcum, T. spinulosum, T. spontaneum, T. squarrosum, T. striatum, T. strictum, T. strigosum, T. subaristatum, T. subsecundum, T. subtile, T. subulatum, T. sunpani, T. supinum, T. sylvaticum, T. sylvestre, T. syriacum, T. tanaiticum, T. tauri, T. tauschii, T. tenax, T. tenellum, T. tenue, T. tenuiculum, T. teretiflorum, T. thaoudar, T. tiflisiense, T. timococcum, T. timonovum, T. timopheevi, T. timopheevii, T. tomentosum, T. tournefortii, T. trachycaulon, T. trachycaulum, T. transcaucasicum, T. triaristatum, T. trichophorum, T. tricoccum, T. tripsacoides, T. triunciale, T. truncatum, T. tumonia, T. turanicum, T. turcomanicum, T. turcomanieum, T. turgidum, T. tustella, T. umbellulatum, T. uniaristatum, T. unilaterale, T. unioloides, T. urartu, T. vagans, T. vaginans, T. vaillantianum, T. variabile, T. variegatum, T. varnense, T. vavilovi, T. vavilovii, T. velutinum, T. ventricosum, T. venulosum, T. villosum, T. violaceum, T. virescens, T. volgense, T. vulgare, T. youngii, T. zea, T. zhukovskyi);

rice (e.g., Oryza abnensis, O. abromeitiana, O. alta, O. angustifolia, O. aristata, O. australiensis, O. barthii, O. brachyantha, O. breviligulata, O. carinata, O. caudata, O. Ciliata, O. clandestina, O. coarctata, O. collina, O. communissima, O. cubensis, O. denudata, O. dewildemani, O. eichingeri, O. elongata, O. fatua, O. filiformis, O. formosana, O. glaberrima, O. glaberi, O. glaberrima, O. glaberrina, O. glauca, O. glumaepatula, O. glutinosa, O. grandiglumis, O. granulata, O. guineensis, O. hexandra, O. hybrid, O. indandamanica, O. jeyporensis, O. latifolia, O. leersioides, O. linnaeus, O. longiglumis, O. longistaminata, O. madagascariensis, O. malampuzhaensis, O. manilensis, O. marginata, O. meijeriana, O. meridionalis, O. meridonalis, O. mexicana, O. meyeriana, O. mezii, O. minuta, O. monandra, O. montana, O. mutica, O. neocaledonica, O. nepalensis, O. nigra, O. nivara, O. officinalis, O. oryzoides, O. palustris, O. paraguayensis, O. parviflora, O. perennis, O. perrieri, O. platyphyla, O. plena, O. praecox, O. prehensilis, O. pubescens, O. pumila, O. punctata, O. repens, O. rhizomatis, O. ridleyi, O. rubra, O. rubribarbis, O. rufipogon, O. sativa, O. schlechteri, O. schweinfurthiana, O. segetalis, O. sorghoidea, O. sorghoides, O. spontanea, O. stapfii, O. stenothyrsus, O. subulata, O. sylvestris, O. tisseranti, O. tisserantii, O. triandra, O. triticoides, O. ubanghensis);

soybean (i.e., Glycine max); barley (i.e., Hordeum vulgare); sugar beet (i.e., Beta vulgaris); hay and fodder crops.

Procedures for transforming plants are well known and routine in the art and are described throughout the literature. Non-limiting examples of methods for transformation of plants include transformation via bacterial-mediated nucleic acid delivery (e.g., via Agrobacteria), viral-mediated nucleic acid delivery, silicon carbide or nucleic acid whisker mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation, sonication, infiltration, PEG-mediated nucleic acid uptake, as well as any other electrical, chemical, physical (mechanical) and/or biological mechanism that results in the introduction of nucleic acid into the plant cell, including any combination thereof. General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell. Mol. Biol. Lett. 7:849-858 (2002)).

Thus, in some particular embodiments, the introducing into a plant, plant part, plant organ and/or plant cell is via bacterial-mediated transformation, particle bombardment transformation, calcium-phosphate-mediated transformation, cyclodextrin-mediated transformation, electroporation, liposome-mediated transformation, nanoparticle-mediated transformation, polymer-mediated transformation, virus-mediated nucleic acid delivery, whisker-mediated nucleic acid delivery, microinjection, sonication, infiltration, polyethylene glycol-mediated transformation, any other electrical, chemical, physical and/or biological mechanism that results in the introduction of nucleic acid into the plant, plant part and/or cell thereof, or a combination thereof.

Agrobacterium-mediated transformation is a commonly used method for transforming plants, in particular, dicot plants, because of its high efficiency of transformation and because of its broad utility with many different species. Agrobacterium-mediated transformation typically involves transfer of the binary vector carrying the foreign DNA of interest to an appropriate Agrobacterium strain that may depend on the complement of vir genes carried by the host Agrobacterium strain either on a co-resident Ti plasmid or chromosomally (Uknes et al. (1993) Plant Cell 5:159-169). The transfer of the recombinant binary vector to Agrobacterium can be accomplished by a triparental mating procedure using Escherichia coli carrying the recombinant binary vector, a helper E. coli strain that carries a plasmid that is able to mobilize the recombinant binary vector to the target Agrobacterium strain. Alternatively, the recombinant binary vector can be transferred to Agrobacterium by nucleic acid transformation (Hofgen & Willmitzer (1988) Nucleic Acids Res. 16:9877). Transformation of a plant by recombinant Agrobacterium usually involves co-cultivation of the Agrobacterium with explants from the plant and follows methods well known in the art. Transformed tissue is regenerated on selection medium carrying an antibiotic or herbicide resistance marker between the binary plasmid T-DNA borders.

Another method for transforming plants, plant parts and plant cells involves propelling inert or biologically active particles at plant tissues and cells. See, e.g., U.S. Pat. Nos. 4,945,050; 5,036,006 and 5,100,792. Generally, this method involves propelling inert or biologically active particles at the plant cells under conditions effective to penetrate the outer surface of the cell and afford incorporation within the interior thereof. When inert particles are utilized, the vector can be introduced into the cell by coating the particles with the vector containing the nucleic acid of interest.

Alternatively, a cell or cells can be surrounded by the vector so that the vector is carried into the cell by the wake of the particle. Biologically active particles (e.g., a dried yeast cell, a dried bacterium or a bacteriophage, each containing one or more nucleic acids sought to be introduced) also can be propelled into plant tissue. Thus, in particular embodiments of the present invention, a plant cell can be transformed by any method known in the art and as described herein and intact plants can be regenerated from these transformed cells using any of a variety of known techniques. Plant regeneration from plant cells, plant tissue culture and/or cultured protoplasts is described, for example, in Evans et al. (Handbook of Plant Cell Cultures, Vol. 1, MacMilan Publishing Co. New York (1983)); and Vasil I. R. (ed.) (Cell Culture and Somatic Cell Genetics of Plants, Acad. Press, Orlando, Vol. I (1984), and Vol. II (1986)). Methods of selecting for transformed, transgenic plants, plant cells and/or plant tissue culture are routine in the art and can be employed in the methods of the invention provided herein. Likewise, the genetic properties engineered into the transgenic seeds and plants, plant parts, and/or plant cells of the present invention described above can be passed on by sexual reproduction or vegetative growth and therefore can be maintained and propagated in progeny plants. Generally, maintenance and propagation make use of known agricultural methods developed to fit specific purposes such as harvesting, sowing or tilling. A nucleotide sequence therefore can be introduced into the plant, plant part and/or plant cell in any number of ways that are well known in the art. The methods of the invention do not depend on a particular method for introducing one or more nucleotide sequences into a plant, only that they gain access to the interior of at least one cell of the plant. Where more than one nucleotide sequence is to be introduced, the respective nucleotide sequences can be assembled as part of a single nucleic acid construct/molecule, or as separate nucleic acid constructs/molecules, and can be located on the same or different nucleic acid constructs/molecules. Accordingly, the nucleotide sequences can be introduced into the cell of interest in a single transformation event, in separate transformation events, or, for example, in plants, as part of a breeding protocol. In some embodiments of this invention, the introduced nucleic acid molecule may be maintained in the plant cell stably if it is incorporated into a non-chromosomal autonomous replicon or integrated into the plant chromosome(s). Alternatively, the introduced construct may be present on an extra-chromosomal non-replicating vector and be transiently expressed or transiently active. Whether present in an extra-chromosomal non-replicating vector or a vector that is integrated into a chromosome, the nucleic acid molecule can be present in a plant expression construct.

### 7. Production and Characterization of Differentiated Plants

7.1 Regeneration

The methods used to regenerate transformed cells into differentiated plants are not critical to this invention, and any method suitable for a target plant can be employed. Normally, a plant cell is regenerated to obtain a whole plant following a transformation or genetic modification process.

Regeneration from protoplasts varies from species to species of plants, but generally a suspension of protoplasts is first made. In certain species, embryo formation can then be induced from the protoplast suspension, to the stage of ripening and germination as natural embryos. The culture media will generally contain various amino acids and hormones, necessary for growth and regeneration. Examples of hormones utilized include auxins and cytokinins. It is sometimes advantageous to add glutamic acid and proline to the medium, especially for such species as corn and alfalfa. Efficient regeneration will depend on the medium, on the genotype, and on the history of the culture. If these variables are controlled, regeneration is reproducible. Regeneration also occurs from plant callus, explants, organs or parts. Transformation can be performed in the context of organ or plant part regeneration as, for example, described in Methods in Enzymology, Vol. 118 and Klee et al. (1987, Annual Review of Plant Physiology, 38:467), which are incorporated herein by reference. Utilizing the leaf disk-transformation-regeneration method of Horsch et al. (1985, Science, 227:1229, incorporated herein by reference), disks are cultured on selective media, followed by shoot formation in about 2-4 weeks. Shoots that develop are excised from calli and transplanted to appropriate root-inducing selective medium. Rooted plantlets are transplanted to soil as soon as possible after roots appear. The plantlets can be repotted as required, until reaching maturity.

In vegetatively propagated crops, the mature transgenic plants are propagated by the taking of cuttings or by tissue culture techniques to produce multiple identical plants. Selection of desirable transgenics is made and new varieties are obtained and propagated vegetatively for commercial use.

In seed propagated crops, the mature transgenic plants can be self-crossed to produce a homozygous inbred plant. The inbred plant produces seed containing the newly introduced foreign gene(s). These seeds can be grown to produce plants that would produce the selected phenotype, e.g., early flowering.

Parts obtained from the regenerated plant, such as flowers, seeds, leaves, branches, fruit, and the like are included in the invention, provided that these parts comprise cells that have been transformed as described. Progeny and variants, and mutants of the regenerated plants are also included within the scope of the invention, provided that these parts comprise the introduced nucleic acid sequences.

It will be appreciated that the literature describes numerous techniques for regenerating specific plant types and more are continually becoming known. Those of ordinary skill in the art can refer to the literature for details and select suitable techniques without undue experimentation.

7.2 Characterization

A population of plants can be screened and/or selected for those members of the population that have a genetic modification. To confirm the presence of the genetic modification in the regenerating plants, a variety of assays may be performed. Such assays include, for example, “molecular biological” assays well known to those of skill in the art, such as Southern and Northern blotting and PCR; a protein expressed by the heterologous DNA may be analysed by western blotting, high performance liquid chromatography or ELISA (e.g., nptII) as is well known in the art.

In some embodiments, a probe is used to determine the presence of a nucleic acid construct of the invention in the genome of a regenerating plant. In other embodiments, the plant genome is analyzed for genetic modification (e.g., SUS2 inactivation) by sequence analysis. In still other embodiments, a genetic modification (e.g., SUS2 inactivation) is analyzed using an antibody that is immuno-interactive with a SUS2 polypeptide.

Representative examples of various methods applicable to characterization of transgenic plants are provided in Chapters 9 and 11 of PLANT MOLECULAR BIOLOGY A Laboratory Manual Ed. M. S. Clark (Springer-Verlag, Heidelberg, 1997).

A population of plants also can be screened and/or selected for those members of the population that have a trait or phenotype (e.g., an increased concentration or yield of sucrose or sucrose derivatives, as compared to a control plant) conferred by the genetic modification. Physical and biochemical methods can be used to identify modified nucleic acids (e.g., introduction of a nucleic acid construct of the invention, or inactivation of SUS2) in the genome of a plant and/or SUS2 expression levels. Selection and/or screening can be carried out over one or more generations, and/or in more than one geographic location. In some cases, plants can be grown and selected under conditions which induce a desired phenotype or are otherwise necessary to produce a desired phenotype in a modified plant. In addition, selection and/or screening can be applied during a particular developmental stage in which the phenotype is expected to be exhibited by the plant. Selection and/or screening can be carried out to choose those modified plants having a statistically significant difference in the concentration or yield of sucrose or sucrose derivatives relative to a control plant in which the nucleic acid has not been modified.

In order that the invention may be readily understood and put into practical effect, particular preferred embodiments will now be described by way of the following non-limiting examples.

### EXAMPLES

### Example 1

### Sucrose Synthase Expressions in Sugarcane and its Relationship with Sucrose Accumulation

The present study was carried out to first classify subfamilies of the SUS gene family expressed in sugarcane and their patterns of expression and to subsequently determine any correlation between expression levels of specific subfamilies and sucrose contents in high-versus low-sucrose cultivars from conventional breeding.

In order to classify the SUS subfamilies expressed in sugarcane, the present inventors sought to first identify SUS genes expressed in sorghum, which, in evolutionarily terms, is the closest relative to sugarcane among all monocots (Dillon et al. 2007. Annals of Botany 100(5):975-989). The sorghum genome was analyzed for homologs of six SUS genes expressed in rice (Hirose et al., 2008. Plant Science 174(5):534-543) and in Arabidopsis (Bieniawska et al., 2007. Plant Journal 49(5):810-828). Five sorghum SUS gene homologs identified from this analysis were then used to screen a sugarcane expressed sequence tag (EST) database for SUS homologs expressed in sugarcane. Five homologous SUS gene subfamilies were identified from this screen and the expression profiles of each subfamily were characterized in different tissues and developmental stages, to identify any relationship between the expression patterns and sucrose contents of sugarcane cultivars grown in glasshouse and in the field.

### Results

Analysis of Five SUS Gene Loci in the Sorghum Genome

The nomenclature used herein follows the nomenclature of Hirose et al. (2008, supra) for rice genes SUS1-6. Accordingly, the gene names SbSUS1, SbSUS2, SbSUS3, SbSUS4 and SbSUS5 denote sorghum genes SUS1, SUS2, SUS3, SUS4 and SUS5, respectively and SoSUS1, SoSUS2, SoSUS3, SoSUS4 and SoSUS5 denote sugarcane genes SUS1, SUS2, SUS3, SUS4 and SUS5, respectively.

Blasting the sorghum genome sequence in Phytozome (http://www.phytozome.net/search.php?method=Org_Sbicolor) with cDNA sequences of the six SUS genes from rice (Hirose et al., 2008, supra) identified 5 loci distributed over three chromosomes (Table 1).

TABLE 1
Chromosome distributions of sorghum, maize, millet, purple false brome and
poplar putative SUS genes corresponding to relative rice genes.

| | | |
|---|---|---|
| Sorghum bicolor | Zea mays | Setaria italica |

| Rice | Gene | X#¹ | Gene code² | Gene | X#¹ | Gene code² | Gene | X#¹ | Gene code² |
|---|---|---|---|---|---|---|---|---|---|
| SUS1 | SbSUS1 | 1 | sb01g033060 | ZmSUS1 | 9 | GRMZM2G152908_T01 | SiSUS1 | 9 | Si034282m.g |
| SUS2 | SbSUS2 | 10 | sb10g006330 | ZmSUS2 | 9 | GRMZM2G089713_T04 | SiSUS2 | 9 | Si005859m.g |
| SUS3 | SbSUS3 | 1 | sb01g033060 | ZmSUS1 | 9 | GRMZM2G152908_T01 | SiSUS1 | 9 | Si034282m.g |
| SUS4 | SbSUS4 | 1 | sb01g035890 | ZmSUS4 | 1 | GRMZM2G318780_T02 | SiSUS4 | 9 | Si034293m.g |
| SUS5 | SbSUS5 | 10 | sb10g031040 | ZmSUS5 | 5 | GRMZM2G060659_T02 | SiSUS5 | 1 | Si020148m.g |
| SUS6 | SbSUS6 | 4 | sb04g038410 | ZmSUS6 | 4 | GRMZM2G045171_T01 | SiSUS6 | 1 | Si005845m.g |
| SUS5 | | | | | | | SiSUS5 | 4 | Si020148m.g |

| | |
|---|---|
| Brachypodium distachyon | Populus trichocarpa |

| Rice | Gene | X#¹ | Gene code² | Gene | X#¹ | Gene code² |
|---|---|---|---|---|---|---|
| SUS1 | BdSUS1 | Bd1 | Bradi1g60320 | PtSUS1 | 18 | POPTR_0018s07380 |
| SUS2 | BdSUS2 | Bd1 | Bradi1g46670 | PtSUS2 | 6 | POPTR_0006s13900 |
| SUS3 | BdSUS3 | Bd1 | Bradi1g20890 | PtSUS3 | 6 | POPTR_0006s13900 |
| SUS4 | BdSUS4 | Bd1 | Bradi1g62957 | PtSUS4 | 2 | POPTR_0002s19210 |
| SUS5 | BdSUS5 | Bd1 | Bradi1g29570 | PtSUS5 | 15 | POPTR_0015s05540 |
| SUS6 | BdSUS6 | Bd3 | Bradi3g60687 | PtSUS6 | 17 | POPTR_0017s02060 |
| SUS5 | | | | PtSUS5 | 12 | POPTR_0012s03420 |
| SUS5 | | | | PtSUS5 | 4 | POPTR_0004s07930 |

¹X#: The chromosome number on which each SUS gene is located.
²The gene codes in the Phytozome of Joint Genome Institute (http://www.phytozome.net).

Rice SUS1 and SUS3 blasted out the same locus on sorghum chromosome 1. Each of the chromosome 1 or 10 has two loci of SUS genes, and chromosome 4 has only one locus. Blasting another two C4 plant genomes of maize and millet with the cDNA sequences of the six SUS genes from rice also showed rice SUS1 and SUS3 match the same locus on maize or millet chromosome 9 (Table 1). Millet has two SiSUS5 loci: one is on chromosome 1, another on chromosome 4 (Table 1). In clear contrast, blasting other sequenced C3 plant genomes showed that they have both SUS1 and SUS3 loci located either on the same or different chromosomes (Table 1).

SUS3, not SUS1, is Lost in all the Sequenced C4 Plants

Using the rice SUS1 and SUS3 to BLAST either the sugarcane or maize EST database, same EST populations and similar score orders of the tentative consensus (TC) sequences were searched out but the results showed higher scores for SUS1 than that of SUS3 (Table 2). Aligning the rice SUS1 or SUS3 protein with the putative polypeptide in either sorghum, maize or millet showed SUS1 has higher similarities and identities than that of SUS3 (Table 3).

TABLE 2
Blasting scores on sugarcane or maize EST and tentative
consensus database by rice sucrose gene SUS1 and SUS3.

Rice

| | SUS1 | SUS3 |
|---|---|---|
| Sugarcane TC123316 | 10164 | 9208 |
| Maize TC549963 | 9784 | 9011 |

TABLE 3
Similarity/identity between rice SUS1 or SUS3 and corresponding
putative SUS proteins from Sorghum, maize and millet.

| | Sorghum | Maize | Millet |
|---|---|---|---|
| Rice SUS1 | 96.9/95.3 | 97.3/95.2 | 97.2/95.3 |
| Rice SUS3 | 94.0/89.8 | 94.0/90.0 | 93.90/90.0 |

Characteristics of Sorghum Putative SUS Proteins

A search for conserved domains of functional motifs on the deduced peptide sequences in the InterPro databases (http://www.ebi.ac.uk/interpro) revealed that the polypeptide product of each of the SbSUS genes has both a SUS domain and a glycosyltransferase domain. This is a typical feature of the SUS protein sequences (Salerno and Curatti 2003. Trends in Plant Science 8(2):63-69).

Properties of the 5 predicted sorghum SUS proteins are shown in Table 4.

TABLE 4
Comparison of the predicted sorghum SUS proteins as deduced
from their cDNA sequences.

| | | Size | | | Identity/similarity (%) | | | | |
|---|---|---|---|---|---|---|---|---|---|
| Gene | Isoform | a.a. | kDa | pl | SbSUS1 | SbSUS2 | SbSUS4 | SbSUS5 | SbSUS6 |
| SbSUS1 | SbSUS1 | 816 | 93.061 | 6.04 | | 87.3 | 79.1 | 64.1 | 58.8 |
| SbSUS2 | SbSUS1 | 802 | 91.815 | 5.82 | 78.8 | | 80.7 | 65.0 | 60.2 |
| SbSUS4 | SbSUS1 | 809 | 92.839 | 6.38 | 68.5 | 69.5 | | 66.0 | 62.7 |
| SbSUS5 | SbSUS5 | 855 | 97.870 | 8.05 | 53.0 | 52.9 | 54.2 | | 78.1 |
| SbSUS6 | SbSUS5 | 838 | 95.219 | 8.46 | 47.6 | 48.7 | 49.9 | 71.2 | |

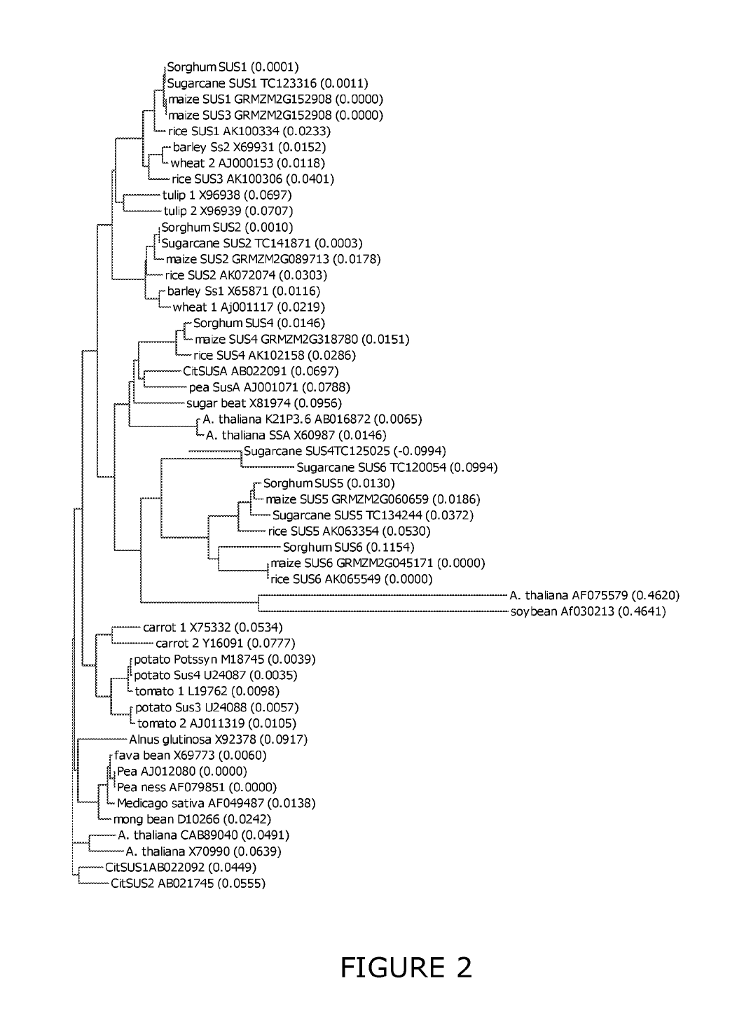

As can be seen from Table 4, peptide identities between rice and sorghum for each sucrose synthase gene are more than 90%; whereas the peptide similarities between sucrose synthase genes within a species are less than 90%. Relatively high levels of similarities exist between the predicted amino acid sequences of SUS1, 2 and 4. SbSUS5 and 6 had lower levels of similarity to SbSUS1, 2 or 4, but showed a higher level of similarity between one another. Based on their molecular sizes, predicted isoelectrical points, and similarity, the five sorghum SUS peptides can be classified into two isoforms, namely SbSUS1 (SbSUS1, 2 and 4) and SbSUS5 (SbSUS5 and SbSUS6) according to (Komatsu et al., 2002. Journal of Experimental Botany 53(366):61-71) and (Hirose et al., 2008, supra). However, a multiple sequence alignment of the deduced peptides from the five sorghum SUS genes with peptides from other plant species indicated that the SbSUS4 might be an individual isoform (FIG. 2).

ESTs and TCs Related to Sugarcane SUS Genes in Database were Classified and Analyzed

The sugarcane genome has not been sequenced. However, a sugarcane database is available, which comprises 282,683 ESTs with 42,377 TC sequences from 28 cDNA libraries. These libraries cover different organ/tissues (root, stem, leaf, inflorescence and seeds) and various developmental stages. The sugarcane EST database (http://compbio.dfci.harvard.edu/cgi-bin/tgi/gimain.pl?gudb=s_officinarum) was searched by using each of the 5 transcript sequences of the sorghum SUS genes so a general picture of the expression of SUS genes in sugarcane could be obtained. Each subfamily of ESTs and TCs identified in the sugarcane database with high homology to a sorghum SUS gene (>90% identity) was listed as a corresponding SoSUS member. Table 5 shows the outcomes from the BLASTing.

TABLE 5
Sugarcane ESTs and TCs blasted out from DFCI Sugarcane Gene Index
(http://compbio.dfci.harvard.edu/cgi-bin/tgi/gimain.pl?gudb=s_officinarum)
by sorghum SUS genes.

| Corresponding to | Sugarcane EST (>90% identity to sorghum) | | | Number of sugarcane TCs | |
|---|---|---|---|---|---|
| sorghum gene | SUS number | %*10⁻² total | % of SUS EST | >90% identity | 60-90% identity |
| SbSUS1 | 534 | 18.89 | 66.42 | 35 | 38 |
| SbSUS2 | 222 | 7.85 | 27.61 | 15 | 57 |
| SbSUS4 | 27 | 0.01 | 3.36 | 4 | 113 |
| SbSUS5 | 18 | 0.006 | 2.24 | 2 | 55 |
| SbSUS6 | 3 | 0.001 | 0.37 | 1 | 172 |

The SoSUS1 members accounted for two thirds of the total SoSUS ESTs or TCs and the SoSUS2 members for 26-27%. The deduced amino acid sequences from the longest SoSUS TCs in each subfamily, in which SoSUS1, SoSUS2 and SoSUS5 were full length, were selected for multiple sequence alignment (MSA) with SUS genes from other plant species. A phylogenetic dendrogram based on the MSA shows all sugarcane SUSs aligned within the monocots cluster (FIG. 2).

ESTs or TCs belonging to the same SoSUS subfamily were mapped to organs and tissues based on their appearance in different libraries to obtain a general picture of sugarcane SUS expression patterns (Table 6).

SoSUS1 was expressed in almost all libraries across different organs and tissues and different developmental stages, except for developing seeds, mature leaves, mature roots and etiolated leaves. Even though SoSUS2 was less richer compared to SoSUS1 (see, Table 5), it expressed more extensively than SoSUS1 across all tissues with the exception of young inflorescence. Overlapping patterns of SUS genes is typical except for SoSUS6. SoSUS6 has only one TC and three ESTs, appearing only in the stalk bark cDNA library.

Sucrose Synthase Isoforms Differentially Expressed in Glasshouse Grown Sugarcane

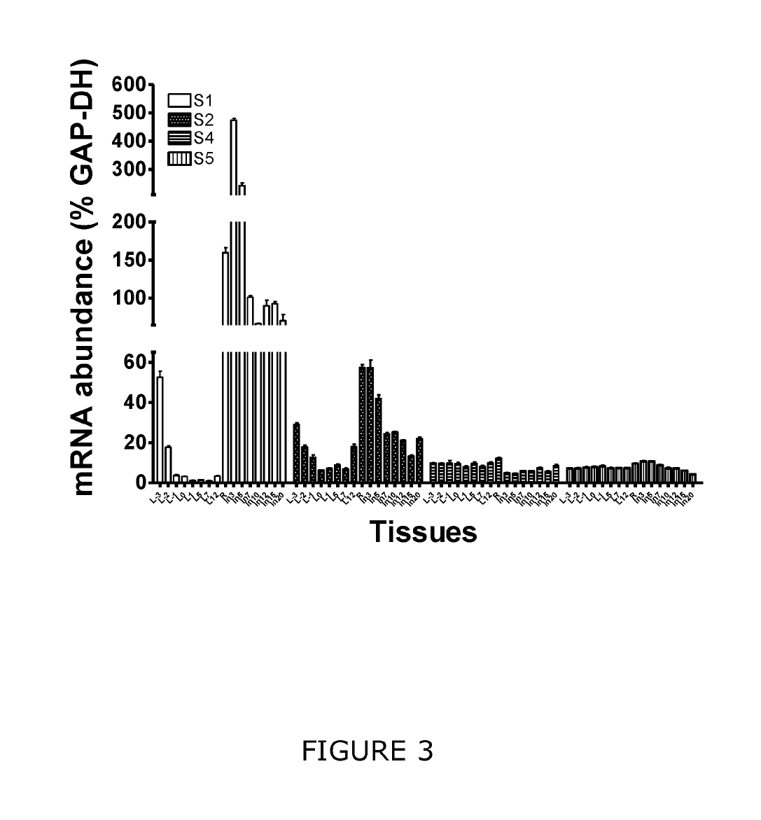

Expression profiles of each SoSUS member were further characterized by RT-qPCR in the elite commercial variety of sugarcane (Q117) grown under glasshouse conditions. SoSUS6 was not detected from the selected material for RNA extraction. FIG. 3 illustrates the expression levels for the remaining four SUS members as normalized to the constitutive GAPDH gene transcript level.

There were relatively small changes in the mRNA pool sizes of the SoSUS4 and 5 between different tissues and developmental stages. SoSUS1 and 2 not only accumulated high levels of mRNA but also showed large variations at mRNA levels. Sink organs such as elongating internodes, young roots and non-photosynthetic leaf blades presented large pool sizes of SoSUS2 and especially SoSUS1 isoforms. The mRNA amount of SoSUS1 was still high in mature stem tissues.

Expression of Sucrose Synthase Genes were Differentially Reduced in the High-CCS Stem Tissues

SUS mRNA profiles were compared between two populations of sugarcane plants with high- vs. low-CCS cultivars to determine if any relationship exists between sucrose accumulation and SUS gene expression. Table 7 illustrates detailed sucrose contents at different developmental stages.

TABLE 7
Sucrose contents in stem tissues of the 4 high-CCS and 4 low-CCS lines.
The samples were from 9 month old ratoons grown in the field.
Values in the large panel are means of 3 reps ± SE.

| | Sucrose content in cultivars (mM) | | | | | | | |
|---|---|---|---|---|---|---|---|---|
| | High-CCS | | | | Low-CCS | | | |
| Internodes | 5080 | 6493 | 6677 | 6498 | 2599 | 6461 | 6641 | 6765 |
| 3 | 126 ± 13 | 83 ± 12 | 362 ± 16 | 74 ± 21 | 116 ± 15 | 111 ± 12 | 53 ± 9 | 71 ± 10 |
| 7 | 126 ± 17 | 419 ± 14 | 561 ± 14 | 391 ± 10 | 256 ± 22 | 394 ± 18 | 483 ± 14 | 263 ± 20 |
| 15* | 494 ± 27 | 576 ± 15 | 677 ± 15 | 528 ± 11 | 339 ± 12 | 454 ± 31 | 441 ± 21 | 430 ± 28 |

*There is a significant difference (P < 0.05) in sucrose contents between means of high- and low-CCS cultivars in internode15 by nonparametric t test.

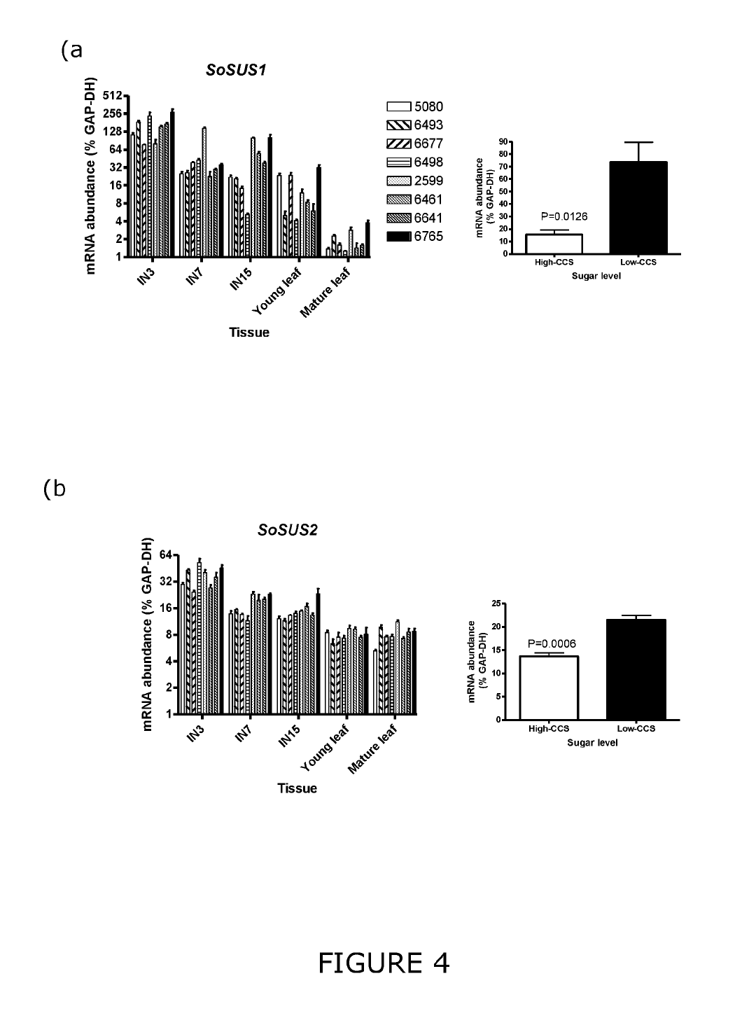

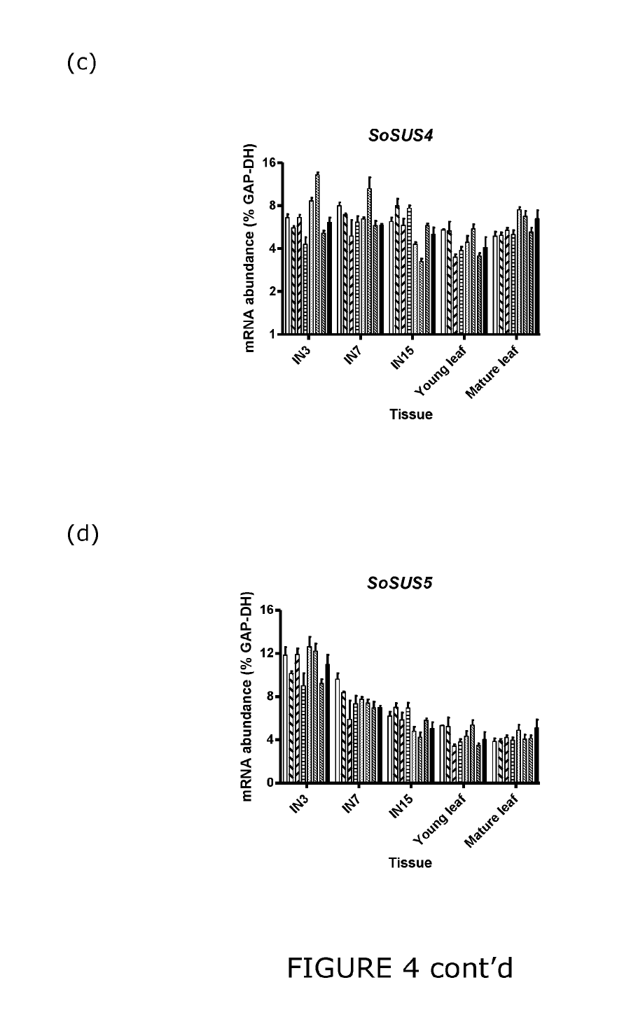

RT-qPCR was performed on the three typical developmental stages along stem and sink/source leaves. SoSUS1, SoSUS2 and SoSUS5 genes were expressed less in leaves than that in stem tissues, especially SoSUS1. There was no significant difference in SoSUS4 between different organs and tissues (see, FIG. 4).

Similar to the data observed from the glasshouse grown sugarcanes, the SoSUS1 accumulated the highest level of transcripts among all tested SoSUS members in stem and young leaf tissues (FIG. 4) of the field samples. More importantly, significant differences were observed between the high- and low-CCS lines in expression of SoSUS1 genes in mature sugarcane internode 15 (FIG. 4a) and in expression of SoSUS2 genes in sucrose peak loading sugarcane internode 7 (P=0.0006) (FIG. 4b).

Significant reduction (P<0.01) in SoSUS1 transcripts was observed from internode #7 to internode #15 in high-CCS group but not in low-CCS group (FIG. 4a). In contrast, significant reductions were observed in SoSUS1 and SoSUS2 transcripts from internode #3 to internode #7 in both high- and low-CCS canes (FIG. 4a,b), which is in agreement with the data from glasshouse grown cane (see, FIG. 3) and also with the young tissue richness of SoSUS1 and SoSUS2 ESTs in database (see Table 5).

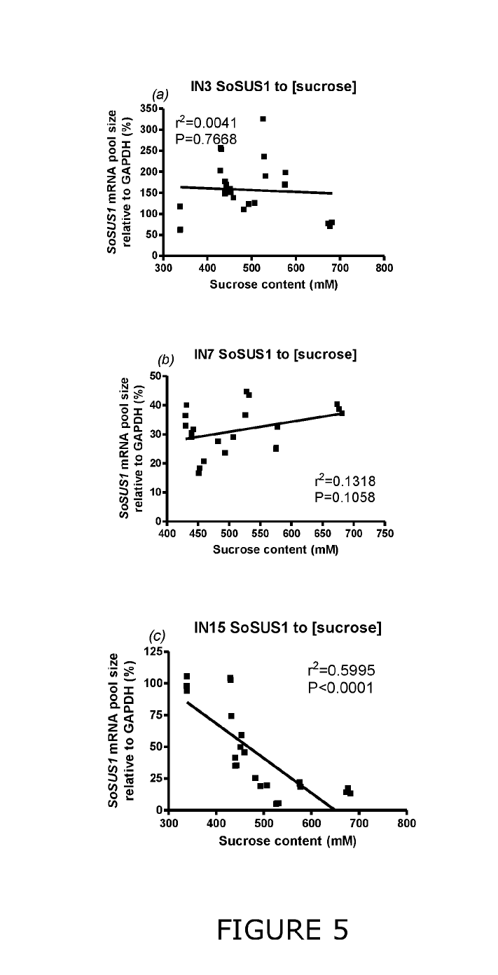

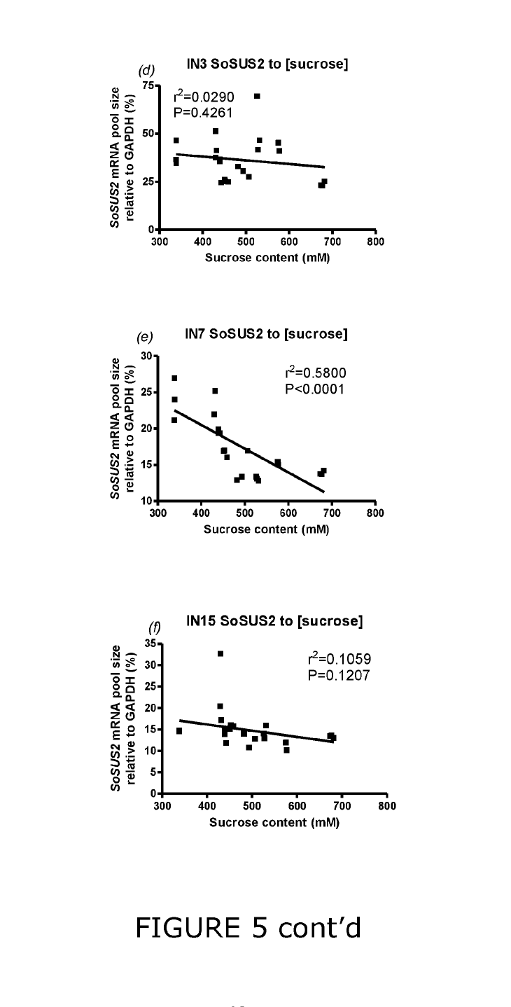

To find out if there is a coarse regulation on sucrose accumulation by SUS transcripts, data of SUS mRNA levels and sucrose content in the field grown sugarcanes were further analyzed. There was a strong correlation (P<0.0001) between SoSUS1 mRNA pool size in internode 15 and sucrose content in whole cane juice (FIG. 5c). The inverse relationship (P<0.0001) was also observed between SoSUS2 mRNA pool size in internode 7 and sucrose content in whole cane juice (FIG. 5e).

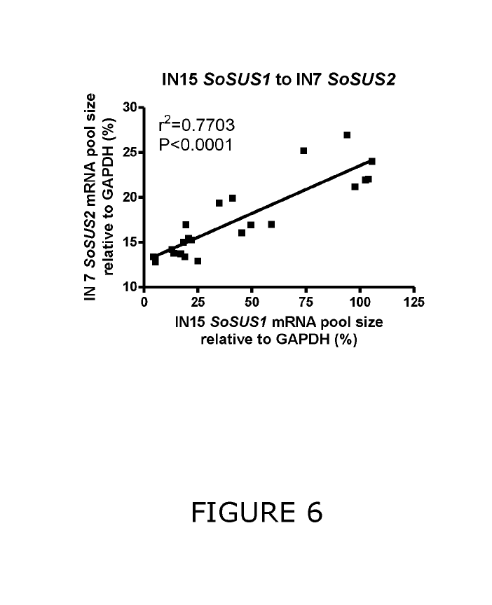

A strong correlation was also observed between SoSUS1 in internode 15 and SoSUS2 transcripts in internode 7 (FIG. 6), implying a coordination between different SUS genes in different developmental stages.

To find out whether the regulation on sucrose accumulation by transcripts of internode 15 SoSUS1 and of internode 7 SoSUS2 is via enzyme, the present inventors further measured SUS activities in cleavage direction (SUS(b)) on the same plant materials (Table 8).

TABLE 8
SUS Activities (breakage) in stem tissues of the 4 high-CCS and 4 low-CCS
cultivars. The samples were from 9 month old ratoons grown in the field.
Values are means of 3 reps ± SE.

| | Enzyme activities (n mol/mg protein/min) | | | | | | | |
|---|---|---|---|---|---|---|---|---|
| | High CCS | | | | Low-CCS | | | |
| Internode | 5080 | 6493 | 6677 | 6498 | 2599 | 6461 | 6641 | 6765 |
| 3 | 19.8 ± 1.2 | 9.8 ± 0.8 | 9.0 ± 0.6 | 15.8 ± 0.7 | 8.1 ± 1.4 | 6.0 ± 0.6 | 9.5 ± 0.6 | 10.0 ± 0.4 |
| 7* | 43.7 ± 1.5 | 53.5 ± 3.1 | 46.0 ± 0.8 | 29.9 ± 1.9 | 121.9 ± 2.9 | 67.8 ± 2.3 | 64.9 ± 10.7 | 71.4 ± 2.4 |
| 15** | 17.8 ± 1.1 | 18.9 ± 15.3 | 20.5 ± 1.0 | 22.1 ± 1.4 | 44.2 ± 2.7 | 29.4 ± 0.9 | 35.2 ± 1.8 | 41.5 ± 2.5 |

* and ** There is a significant difference (*P < 0.05: **P < 0.01) in sucrose contents between high- and low-CCS cultivars in internode 7 or 15, respectively, by nonparametric t test.

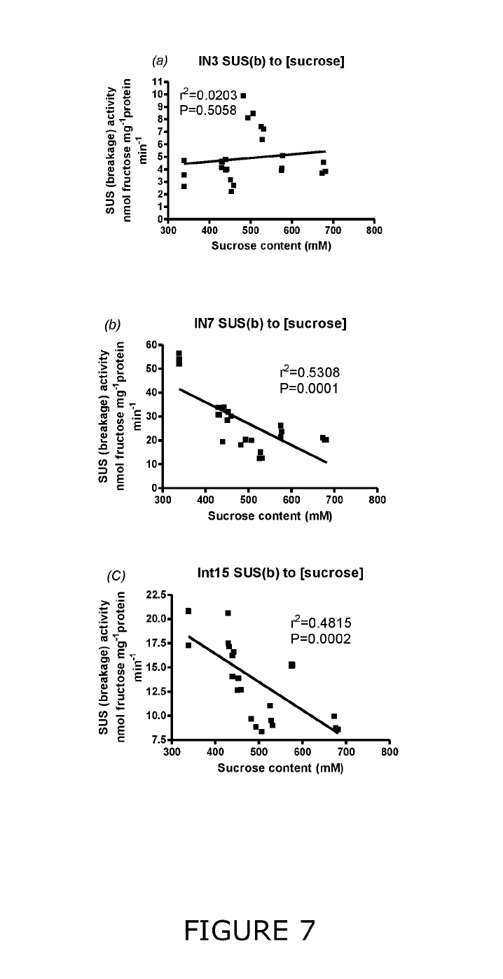

The in vitro activity strength order of #7, #15 then #3 (Table 8) was different from the patterns at SoSUS transcripts (see, FIG. 4). The SUS(b) activity was significantly higher in low-CCS cultivars than in high-CCS cultivars in internodes 7 (P=0.0378) and 15 (P=0.0021). Inverse relationships between sucrose contents in whole cane juice and SUS(b) activities in internode 7 (FIG. 7b) and 15 (FIG. 7c).

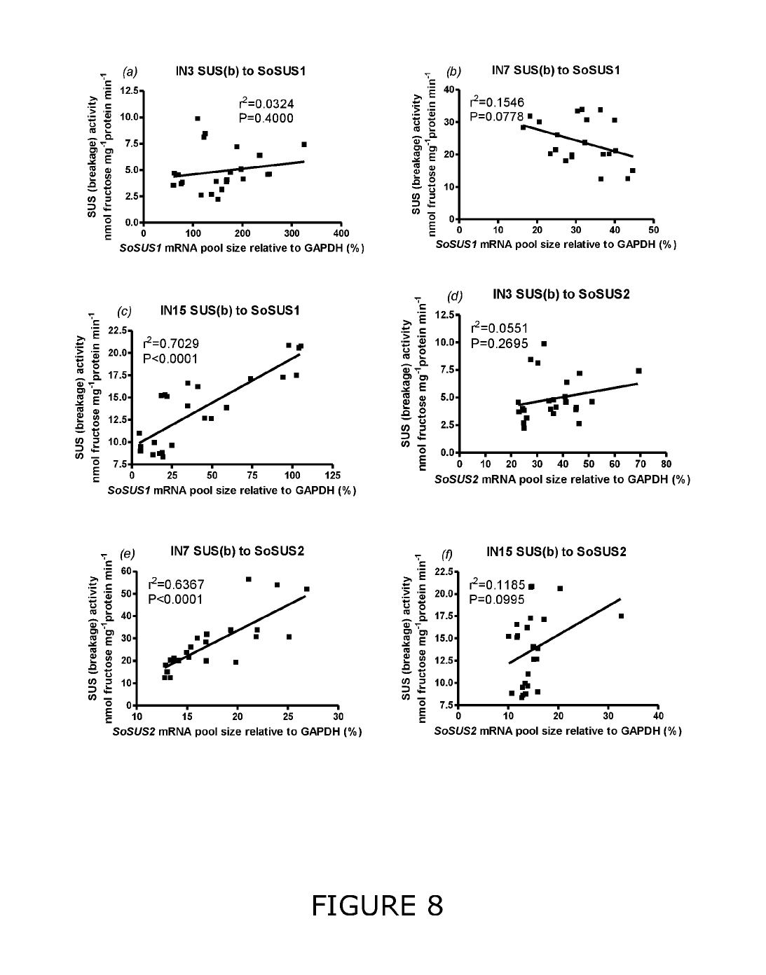

Relationship between transcript level of a specific SoSUS member and SUS(b) activities could also be established in some cases, even though the present inventors did not differentiate the SUS isozyme activity in the current measurements: SoSUS1 transcripts correlated with SUS(b) activity in internode 15 and SoSUS2 with SUS(b) in internode 7 (see, FIG. 8).

### Discussion

Results in the current study indicate that SoSUS1, as the largest mRNA pool size among all SoSUS members, was predominately expressed in sugarcane stem and root tissues, as well as in leaves. This is in agreement with the expression pattern of rice SUS1 (Hirose et al., 2008, supra). Consistent with these results, more than 66% of the total SoSUS ESTs appeared as SoSUS1 in the current sugarcane cDNA database (http://compbio.dfci.harvard.edu/cgi-bin/tgi/gimain.pl?gudb=s_officinarum). While the high CCS canes showed normal growth with good cane yield, SoSUS1 expression levels in this group were significantly reduced. This suggests SoSUS1 expression levels were not high in matured sugarcane stem tissues (FIG. 4a)”

SoSUS2, as the second largest mRNA pool sizes in stem, leaf and root tissue, showed less difference between leaf and stem tissues than SoSUS1 did in the current study. The cDNA database indicates SoSUS2 was expressed in a wide range of tissues, which is agreement with those reported in rice (Wang et al., 1999. Plant and Cell Physiology 40(8):800-807; Hirose et al., 2008, supra). Of the high-CCS lines, in contrast to the SoSUS1 which showed significant difference in expression levels at matured internode 15, SoSUS2 transcript levels showed significant reduction in sucrose loading internode 7 (FIG. 4b).

Gene expression and metabolism could be directly regulated at the transcript level (Mattick 2004. Nature Reviews Genetics 5(4):316-323; Mattick et al., 2009. Bioessays 31(1):51-59). Experimental results in this study imply a transcriptional coarse control on sucrose accumulation via SUS(b) enzyme activities, at least partially. Three pieces of experimental data support this argument: 1) tight associations of sucrose content in whole cane juice with SUS enzyme activity (breakage) in the maturing and matured internodes (see, FIG. 7); 2) strong correlations between SoSUS1 expression level in matured internodes (and between SoSUS2 expression level in maturing sucrose loading internodes) and sucrose contents (see, FIG. 5); 3) coincidence of the significant reductions in mRNA pool sizes of SoSUS1 and SoSUS2 genes in matured and sucrose loading internodes, respectively (see, FIGS. 4a and b and see, FIG. 6). However, the mRNA pool sizes and even in vitro enzyme activities are not measures of flux. More detailed investigation on the roles of the different SUS isoforms encoded by these SUS members on sucrose accumulation will be required.

SoSUS4 and SoSUS5 were expressed at relatively lower levels than SoSUS1 and SoSUS2 in all tested leaves, stems and roots and at all developmental stages (FIGS. 3 and 4). Consistent with these results, the ESTs from these two SoSUS genes together only accounted for 5.6% of the total SUS ESTs (Table 5). These two members did not show any difference between the high- and low-CCS lines (FIGS. 4c and d).

### Materials and Methods

Plant Materials

Sugarcane from Glasshouse:

Sugarcane cultivar Q117 plants were grown in a containment glasshouse under natural light intensity at 28±2° C. with watering twice a day. Each plant was grown as a single stalk in a pot of 20 cm diameter (4 L volume) and sampled as a 9-month-old ratoon. Leaves were numbered from one for the top visual dewlap (TVD), with higher numbers for older leaves. Internodes were numbered according to the leaf attached to the node immediately above. Tissues sampled included non-photosynthetic (spindle) leaves-3 and -2, mature leaf blades (+3) and sections from the middle of internodes 3, 7, and 15. These represented the physiological status of tissue that was sucrose loading and matured, respectively. The roots were sampled between the soil and pots by carefully selecting the white, young tender ones. Stem samples were rapidly cored by a hole-borer and frozen in liquid nitrogen, then transported in liquid nitrogen to the laboratory for analyses of sugars and RNA.

Sugarcane from Conventional Breeding:

Eight lines with similar growth and stalk biomass from two biparental crosses (the following KQ97 from Q117×MQ77-340, n=237; KQ04 from ROC1×Q142, n=300) were selected for the experiment. Four lines with high commercial cane sugar (CCS) (KQ97-5080, KQ04-6493, KQ97-6677, KQ04-6498) and four with low CCS (KQ04-6461, KQ04-6641, KQ97-6765, KQ97-2599) were planted in a field trial with three replicates of one row by 10 m, at Kalamia, North Queensland (19° 32'S, 147° 24′E) on 18 Oct. 2007. Normal commercial agronomic practices were applied. Samples were taken on 20 Jul. 2009, when the ratoon plants were 9 months of age with about 22 internodes. In all sampling, material was pooled from 3 plants per replicate. The numbering of internodes was same as Glasshouse sampling. Stem samples were rapidly cored by a hole-borer and frozen in liquid nitrogen in the field, then transported on dry ice to the laboratory for analyses of sugars, enzymes and RNA. The remainder of the culm from the sampled stalks was crushed using a small mill for juice extraction. Brix was measured on a 300 μl sample of this ‘whole-stalk’ juice using a pocket refractometer (PAL-1, Atago Co. Ltd, Japan) zeroed using MilliQ water prior to each sample.

RNA Extraction and cDNA Synthesis

Frozen plant tissues were ground into fine powder by ball milling (Retsch MM301, Germany). Total RNA was extracted using Trizol following the kit protocol

(Invitrogen). RNA concentration was determined using a Nanodrop ND-1000 (Biolab).

Complementary DNA was prepared from 1 μg samples of total RNA, following the protocol described in the Supercript III first strand synthesis kit (Invitrogen).

Primer Design and RT-qPCR

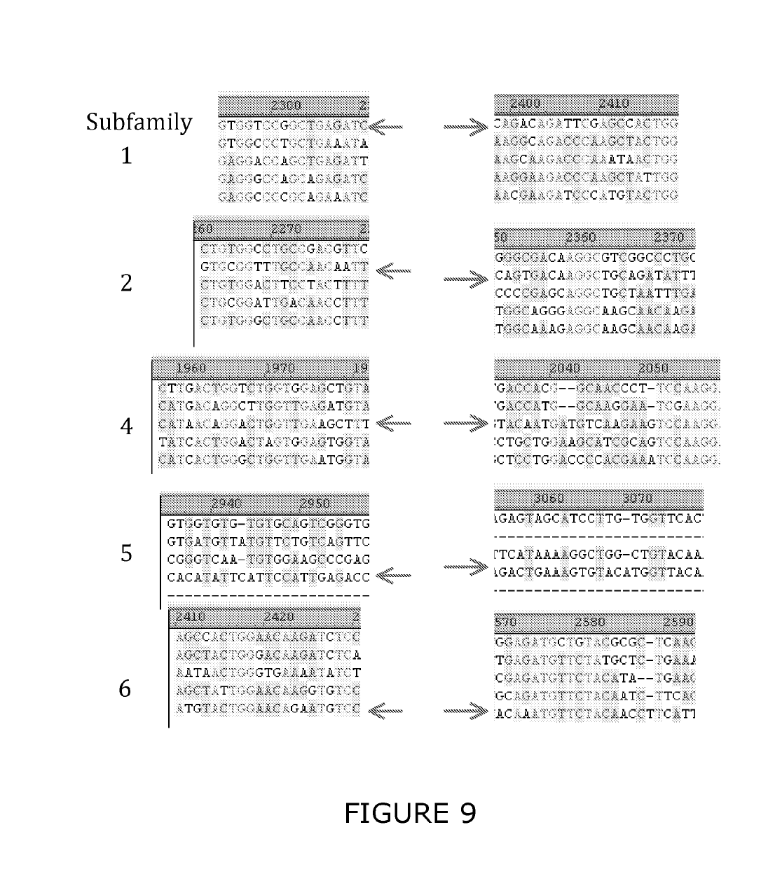

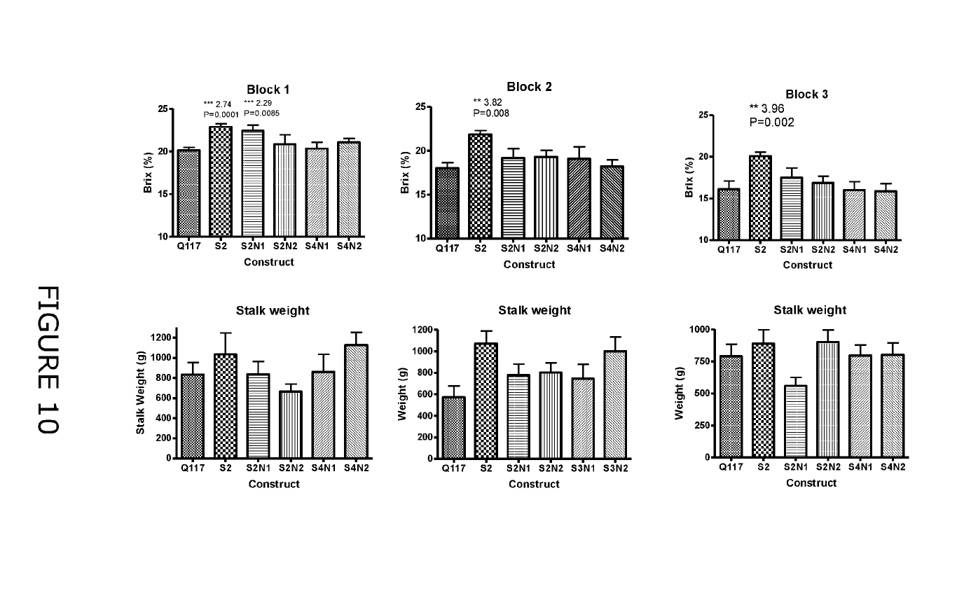

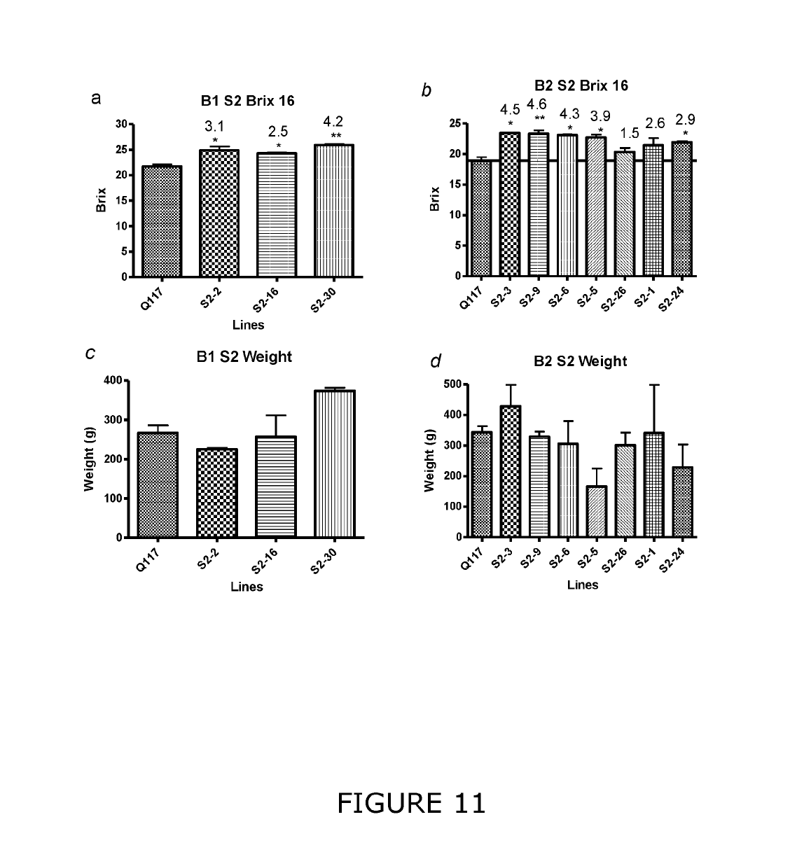

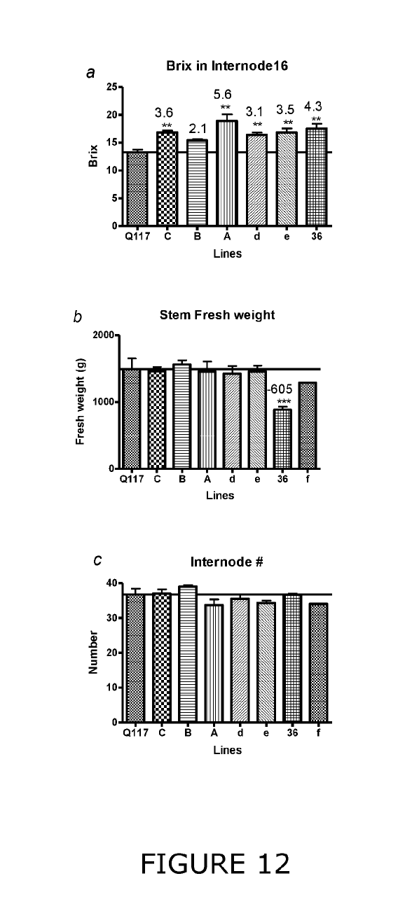

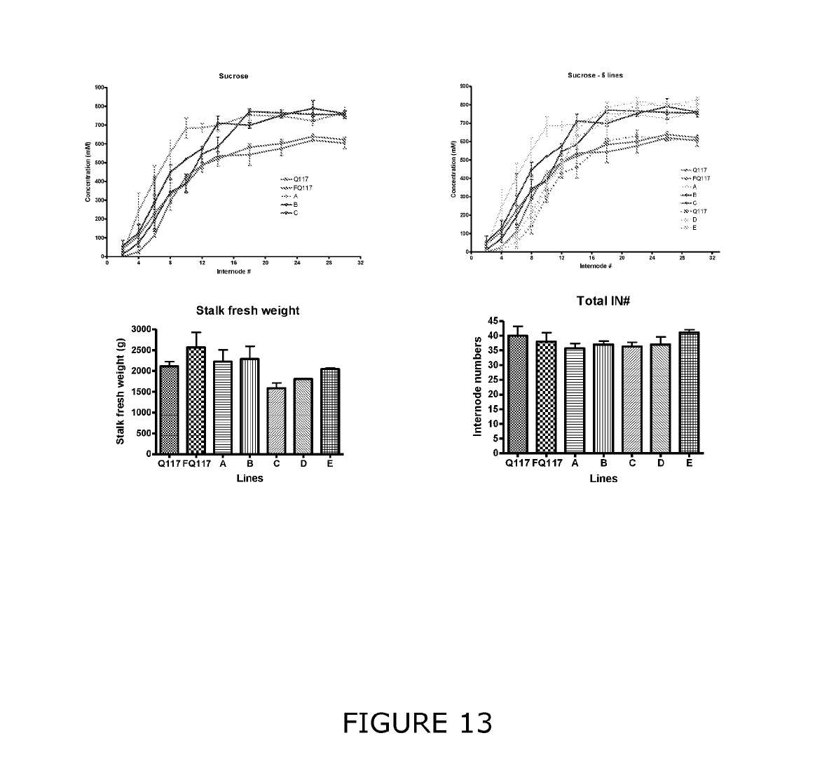

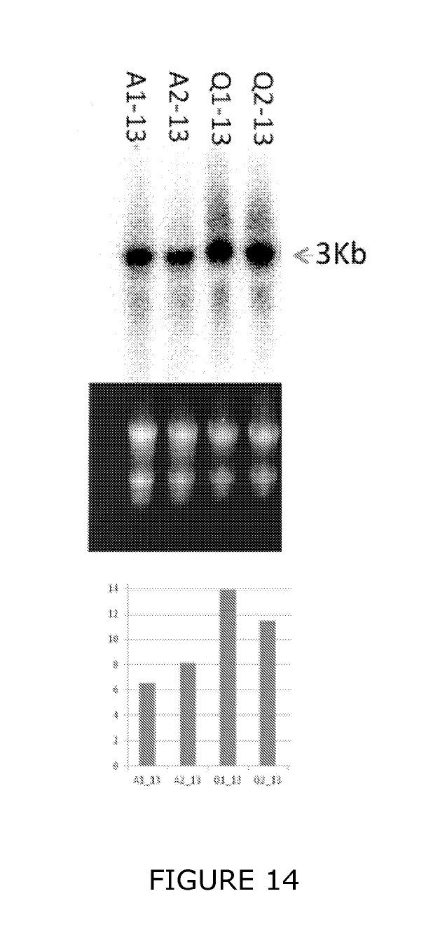

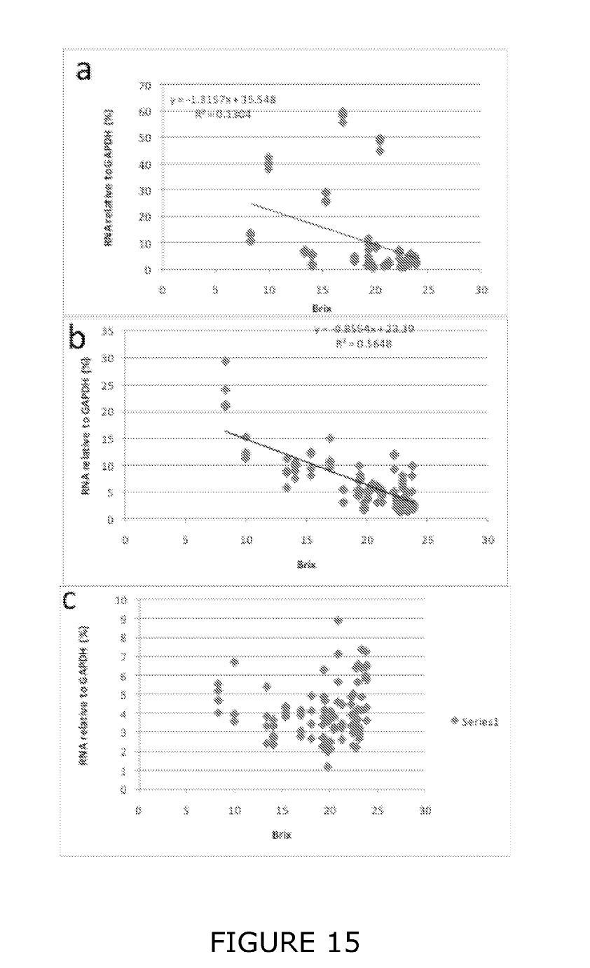

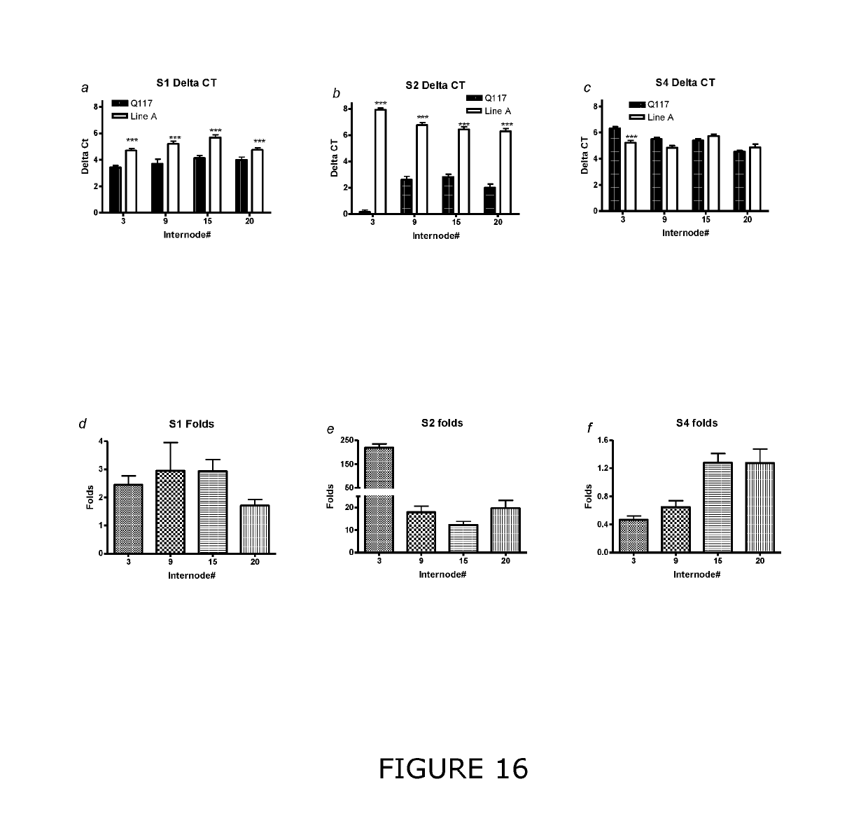

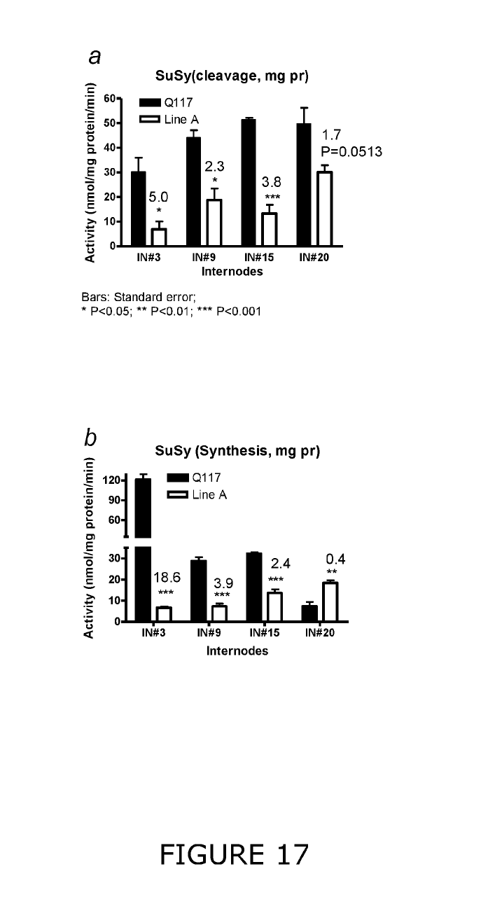

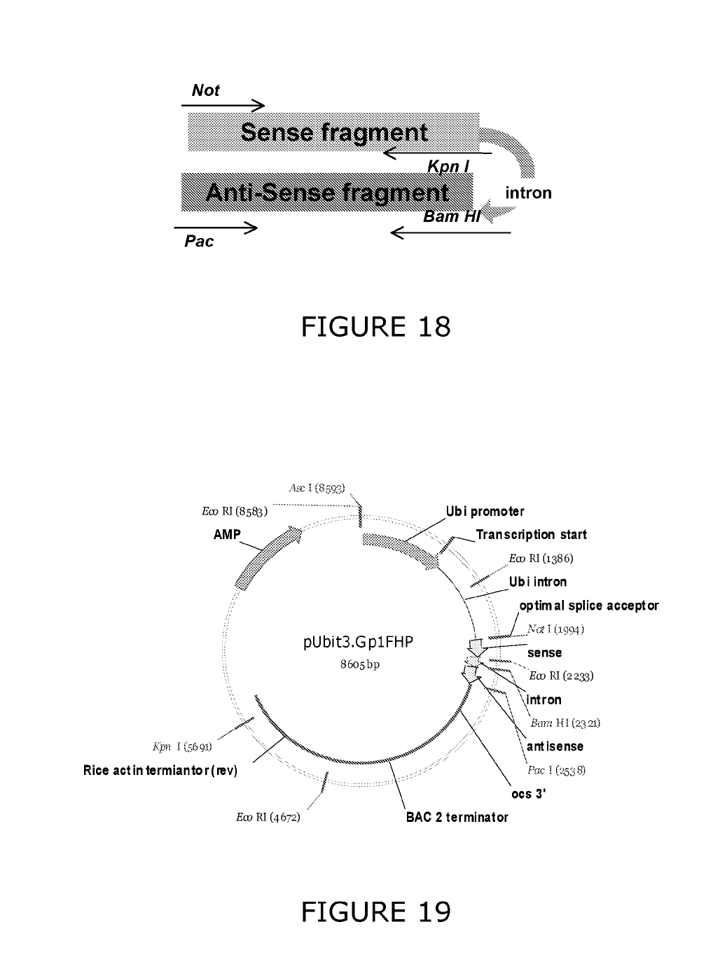

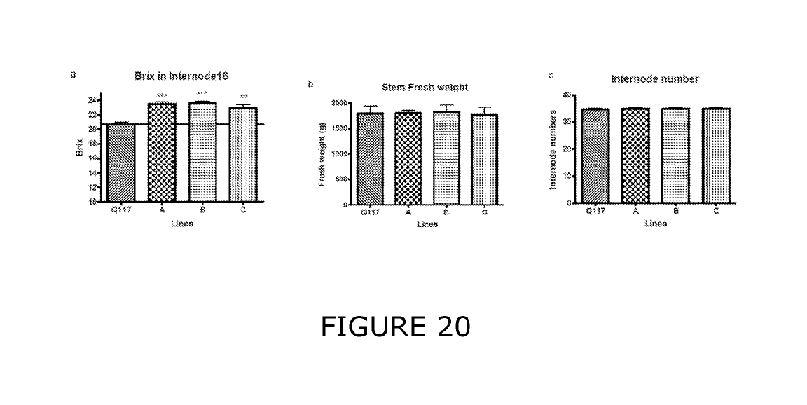

Subfamily-specific and universal within subfamily primers of the sucrose synthase genes for sugarcane were designed. First, the most variable regions were identified along SUS genes from a multiple sequence alignment of the deduced polypeptides of plant sucrose synthases (refer FIG. 2). Then, conserved elements within the identified variable regions for each sugarcane SUS subfamily were further identified. Finally, the variable regions close to the 3′ end of the genes were selected to design primers of subfamily specific (FIG. 9) but universal within each subfamily (Table 8). Mismatched base pairs for each subfamily were generally designed to be located at the 5′ end of the primer and the total was minimized to less than 3% of the total base pairs involved (Table 8). Primer designing principles from the software package Primer Express (Applied Biosystems) were also considered for the five sucrose synthase gene members in sugarcane.

RT-qPCR was run a ABI PRISM® 7900HT Sequence Detection System using an Eppendorf epMotion™ 5075 Workstation. Each 10 μL reaction contained 1× SYBR® Green PCR Master Mix (Applied Biosystems), 200 nM primers and 1:25 dilution of cDNA (from 40 μL cDNA synthesis). The RT-qPCR program was run at 95° C. for 10 min, 45 cycles of 95° C. for 15 sec and 59° C. for 1 min, then dissociation analysis at 95° C. for 2 min and 60° C. for 15 sec ramping to 95° C. for 15 sec. Means from three sub-samples were used for each analyzed cDNA sample.

TABLE 9
SoSUS member specific primers used for RT-qPCR.

| | | | | Mismatch³ |
|---|---|---|---|---|
| Oligo Name | Primer Sequence | ESTs¹ | bps² | (%) |
| SoSUS1 F | TGGTCCGGCTGAGATCATC (SEQ ID NO: 4) | 35 | 665 | 1.8 |
| SoSUS1 R | TCCAGTGGCTCGAATCTGTCTG (SEQ ID NO: 5) | 30 | 660 | 1.4 |
| SoSUS2 F | GTGCGGTTTGCCAACAATT (SEQ ID NO: 6) | 40 | 800 | 3.0 |
| SoSUS2 R | AAATATCTGCAGCCTTGTCACTGT (SEQ ID NO: 7) | 40 | 1000 | 1.9 |
| SoSUS4 F | CATAACAGGACTGGTTGAAGCTTT (SEQ ID NO: 8) | 8 | 200 | 0.5 |
| SoSUS4 R | CCTTGGACTTCTTGACATCATTGTA (SEQ ID NO: 9) | 9 | 234 | 0.4 |
| SoSUS5 F | CACATATTCATTCCATTGAGACC (SEQ ID NO: 10) | 6 | 138 | 0.0 |
| SoSUS5 R | TGTAACCATGTACACTTTCAGTC (SEQ ID NO: 11) | 6 | 138 | 0.0 |
| SoSUS6 F | ATGTACTGGAACAGAATGTCC (SEQ ID NO: 12) | 5 | 105 | 0.0 |
| SoSUS6 R | TGAAGGTTGTAGAACATTTGT (SEQ ID NO: 13) | 5 | 105 | 1.8 |
| GAPDH F | CACGGCCACTGGAAGCA (SEQ ID NO: 14) | | | |
| GAPDH R | TCCTCAGGGTTCCTGATGCC (SEQ ID NO: 15) | | | |

¹Available ESTS on the web sides in each subfamily;
²bps: total base pairs involved = primer length * available ESTs;
³Mismatch (%) = mismatched base pairs/(primer length in base pairs* EST number)

Amplicons were cloned into pCR® 2.1-TOPO® vector (Invitrogen) and multiple products were sequenced to confirm sucrose synthase member specificity.

The reference gene for quantitative PCR was the cytosolic isoform of glyceraldehydes-3-phosphate dehydrogenase (GAPDH) that exhibited stable levels of expression in a broad range of sugarcane tissues (Iskandar et al., 2004. Plant Molecular Biology Reporter 22(4): 325-337).

Crude Enzyme Extraction

Enzymes were extracted by grinding the frozen powder (as for RNA extraction) in a chilled mortar using 3 volumes of extraction buffer that contained 0.1 M Hepes-KOH buffer, pH 7.5, 10 mM MgCl₂, 2 mM EDTA, 2 mM EGTA, 10% glycerol, 5 mM DTT, 2% PVP and 1× complete protease inhibitor (Roche) as detailed (Wu and Birch 2011. Plant Physiology 157:2094-2101). The homogenate was centrifuged at 10,000×g for 15 min at 4° C. The supernatant was immediately desalted on a PD-10 column (GE Healthcare) that was pre-equilibrated and eluted using an extraction buffer without glycerol. This desalted extract was used for enzyme assays. Protein concentration was assayed by the Bradford reaction using a Bio-Rad kit with bovine serum albumin standards.

Sucrose Synthase Assays

Sucrose synthase (breakage) activity was assayed in a reaction mixture comprising 100 mM Tris-HCl buffer pH 7.0, 2 mM MgCl₂, 160 mM sucrose and 2 mM UDP. Blank reactions without UDP were included as an additional negative control. After 30 min at 30° C., the assay was terminated by boiling for 10 min. The fructose product was measured using a BioLC as described below and confirmed based on UDPG levels as described before (Wu and Birch 2011, supra).

Sugar Determination

To measure intracellular glucose, fructose and sucrose, the frozen powder was diluted in 1:20 water (w:w) and then heated for 10 min at 96° C. to inactivate enzymes, centrifuged at 16,795×g for 10 min at 4° C. to remove particulates, and analyzed by HPAEC (Wu and Birch 2007, Plant Biotechnology Journal 5(1):109-117).

BLAST Searches

All sucrose synthase ESTs were obtained from the NCBI database (http://www.ncbi.nhn.nih.gov) and all tentative consensus (TC) sequences were from the Computational Biology and Functional Genomics Laboratory (http://compbio.dfci.harvard.edu/cgi-bin/tgi/gimain.pl?gudb=s_officinarum). Sorghum and rice genomes were blasted on the Phytozome database (http://www.phytozome.net/search.php).

Statistical Analyses

Nonparametric t test and correlation analyses were performed using GraphPad Prism 5.0 software (San Diego, Calif., USA).

### Example 2

### Enhancement of Sucrose Accumulation by Down-Regulating Expression of a Specific Sucrose Synthase Subfamily

In Example 1, the present inventors identified 681SUS ESTs and classified them as five SUS subfamilies by comparison with the fully-sequenced genomes of Arabidopsis, rice and sorghum. Based on this classification, they prepared unique hair-pin constructs so that they could modulate expression of SUS subfamily. Furthermore, research team had previously isolated and characterized a range of promoters (Mudge et al. 2009 Planta 229: 549-558); Osabe 2010 PhD thesis, The University of Queensland), permitting down-regulation of target genes selectively in key tissues, in this case the mature stems. All single gene constructs were transferred into elite sugarcane cultivar Q117.

### Results

Improved Sucrose Content was Observed in SUS2 Down-Regulating Transgenic Lines with High Transformation Rate

Six SUS genes were targeted using hairpins. Thirty transgenic lines in each category were grown in containment glasshouse. From these transformations, SUS2 down-regulation showed significantly enhanced sugar accumulation (P=0.0001), with 3-4 Brix units higher than the control (FIG. 10A-C) in internode 16. The Brix values truly reflect the sucrose concentration in the sugarcane juice, since a tight correlation was found between them. A corresponding 15-21% sucrose was increased compared to the parent control. This result was statistically analyzed across 3 experimental blocks harvested at different Brix values. There was no apparent negative influence on sugarcane growth and development, the means of the stalk fresh weight of the SUS2 down-regulating lines were even higher than the controls on each block (FIGS. 10D-F). There was no correlation between stalk fresh weights and Brix values. Also, there was no significant difference in plant height and node numbers between the sugar-improved transgenic lines and the controls.

The Enhancement Effect on Sucrose Content in SUS2 Down-Regulating Lines was Passed to Second Generation which is Stable at Different Environment and Developmental Stages

To test the stability of the effects of SUS2 gene down-regulation, part of the lines with this gene construct were randomly selected from each block to plant in 2 L-soil pots for second generation test with replications on each line. Planting materials used for the controls also went through the same conditions as the transgenic lines: plantlets came through tissue culture and were moved to glasshouse as first generation. When the second generation transgenic and control plants grew to 5 month old, part of them were left on the bench and the rest were moved into 333 L-soil pots to mimic the field conditions.

Enhancement Effects were Still Shown in SUS2 Down-Regulating Plants of Second Generation Grown in Small Pots

Similar to the first generation, Brix values were increased 2-4 compared to the control in the second generation (FIGS. 11a and b). There was no negative influence on stalk fresh weight, indicating normal growth and development in the transgenic lines (FIGS. 2c and d).

Increased Sugar Content in Main Stalks Grown in the Large Pots when the Canes were not Full Matured.

When plants grew another 6 months in the large pots, the main stalks were sampled for characterization. Similar to the plants grown in small pots, the transgenic lines had 2-5 Brix values higher than the controls, even though all canes showed tender when harvesting (FIG. 12a). No negative influence was found from gene transformation on stalk fresh weight (FIG. 12b) and internode numbers (FIG. 12c).

Increased Sugar Content in Fully Matured Secondary Stalks Grown in the Large Pots

From FIG. 13, the high sucrose content phenotype of the SUS2 down-regulating lines was successfully passed to the secondary stalk of the secondary generation. Two Q117 controls were incorporated in the experimental design. The planting materials in one replicated control were originated from tissue culture and came through the glasshouse growing conditions as the transgenic lines, while the planting materials in another replicated control of Q117 was from field at Ayr, Queensland, Australia. In the first wave, 3 individual lines and both controls from field and glasshouse with 3 replicated stalks each line grown in large pots were sampled for comparison. In the matured internodes, around 25% sucrose content was higher than the controls. Planting materials from field or glasshouse had a similar pattern of sucrose accumulation. Some SUS down-regulating lines showed their early maturing phenotype. For example, Line A had 70% more sucrose than the control in internode 10. A month later, to verify the stability of the high-sucrose phenotype, another 2 transgenic lines transformed with SUS2 down-regulating construct were harvested. Same patterns of high-sucrose accumulation in the transgenic lines related to the Q117 controls were observed in the second wave of sampling (FIG. 13b). Similar to the plants grown in small pots and the main stalk grown in large pots, there was no significant differences between the SUS2 down-regulating lines and the controls in the stalk fresh weights (FIG. 13c) and internode numbers (FIG. 13d).

Molecular and Biochemical Data are Consistent the High-Sucrose Phenotype in the SUS Down-Regulating Lines

All transgenic lines grown in glasshouse were positive through construct specific PCR and Q117 controls were negative in construct specific PCR.

Northern blotting showed reduced expression of i gene in transgenic line with i down-regulating construct compared to the control of Q117 (FIG. 14). The full-length transcripts of SUS bands on the gel appeared intact without much degradation. However, the reduction in SUS transcripts could not distinguish SUS subfamily 2 from SUS subfamily 1 because the probe used for the analysis contains conserved regions for both SUS1 and SUS2. RT-q PCR technique with subfamily specific primers could quantify SUS expression levels of each SUS subfamilies. It is plausible to employ RT-qPCR technique to reflect the functional intact SUS transcript levels for each SUS subfamily.

The expression levels of SUS2 subfamily quantified by RT-qPCR on mature internode 15 of SUS2 down-regulating lines and the controls in the first generation associated negatively with the sugar Brix values (FIG. 15b). In contrast, transcript levels of SUS1 and SUS4 from the same plant materials did not have correlations with the sugar contents (FIGS. 15a and c).

RT-qPCR was conducted on different developmental stages of SUS2 down-regulating Line A on second generation grown in the replicated large pots. Expression levels of SUS2 were reduced 10-20 folds from internode 9 down, whereas SUS2 levels in internode 3 were reduced by more than hundred times relative to Q117 (FIGS. 16b and e). SUS1 transcripts also reduced 1.5-3 times compared to the control of Q117 (FIGS. 16a and d). SUS4 expression increased in internode 3 (FIGS. 16c and f).

SUS enzyme activities on sucrose digestion were reduced in all internodes of SUS2 down-regulating plants (FIG. 17). SUS activities on the direction of sucrose synthesis also were down-regulated. It should be noted that these enzyme activities were measured under standard conditions which is not the same cell physiological status. Based on substrate and product concentrations, the SUS enzyme is considered to conduct mainly in the digestive direction in mature sugarcane culms (Claussen et al., 1985. Phylol. Plant 65: 275-280); Schafer et al., 2004. Eup. J. Biochem 271: 3971-3977).

### CONCLUSIONS

Among the SUS subfamilies, only SUS2 was down-regulated markedly with a concomitant 15% enhancement in sucrose accumulation in matured internodes of sugarcane. The enhancement of sucrose content in the SUS2 down-regulating lines did not negate plant growth and development in tested two generations grown under different conditions. The sugar enhancement from manipulation of an endogenous gene is convincing because:

High rates of increased sucrose accumulation are observed in plants transformed with SUS2 down-regulating gene construct. This result was statistically analyzed across 3 experimental blocks harvested at different Brix values;

The high-sucrose phenotype from the SUS2 manipulation has successfully passed on to second generation and latter tiller stalks. The phenotype is stable in transgenic sugarcane plants of different maturity, grown either in large pots or small pots, with either rich sunlight or shaded conditions;

Patterns of sugar accumulation in all five tested lines showed the same high-sucrose content in the matured internodes, even though some lines also demonstrated early maturing pattern;

Characterizations on molecular and physiological levels showed consistent data with the high-sucrose phenotype;

Genomic PCR showed positive for the gene construct incorporation into the genome of the transgenic lines;

Northern and RT-qPCR experiments demonstrate that SUS2 transcripts are reduced more than 10 fold, and even SUS1 expression was halved in stem tissues of SUS2 down-regulating lines;

A negative correlation between SUS2 transcripts and sucrose contents was observed in mature internodes;

SUS enzyme activity (cleavage) was reduced in whole stalk of the SUS2 down-regulating transgenic lines compared to the controls.

### Materials and Methods

Preparation of Sense and Antisense Fragments from Different SUS Groups

After alignment of available TC sequences assembled from 680 ESTs in GenBank, 5 SUS families were classified as SUS1, SUS2, SUS4, SUS5 and SUS6. Based on the alignments, subfamily specific fragment (but universal with the subfamily) was selected. Since SUS5 and SUS6 are rarely found in the EST sequences, only the sense fragments of SUS1, SUS2 and SUS4 (Table 10) were synthesized into the vector of PUC57 by GeneScript (New Jersey, USA) for the hairpin construct, with the restriction sites of Not 1 and Kpn I were incorporated at the 5′ end and 3′ end respectively. Antisense fragments were amplified by high fidelity fusion polymerase (distributed by NEB), with Bam HI and Pac I incorporated in forward primer and reverse primer, respectively. In addition, the SMSO4 Intron II was amplified, with primers incorporating Kpn I and Bam HI in forward primer and reverse primer, respectively (Table 11). The amplified PCR fragments of antisense and intron were cloned into TOPO2.1 cloning vector (Invitrogen).

TABLE 10
Synthesized sequence with Not I and Kpn I restriction endonuclease sites
(bold) at 5′end and 3′end, respectively.

| Subfamily | Sense sequence |
|---|---|
| SUSI | GCGGCCGCAGCCCTCCAGCAAGTGACCCCGCGGCGGCTGGAGACCTGATGAGCGAAAGGGAGCACTTGG |
| | AGTCGTGTTTTTTTCCTTCCCCTGATCCGGAGGCCAAAAAAGAGTCTGCTTTTCTTCCTAGGCGGCGGGCG |
| | TTCGTTGCTGCTCTTCCCTTCAAGCATTGTTATTACCTTGTCAAGGTCTTGTTCCATCATTGATCCGGGTGTT |
| | GGTTTTAGTAGTCTGATCTGAATTGTTAGTAGTTTGGGTTGAGTCGAGCGGTTGAGAGGGATGTTGGGAC |
| | TTGGCGCCCTTTTCCCTGAAATAAGAGTAGCATCCT |
| | TGTGGTTCACTTTGCACCTGGAATCGATGTTTGCCTCAGGGTACC (SEQ ID NO: 16) |
| SUS2 | GCGGCCGCTGTGGGAAAGAAGAACCCCAATCTGGAGTAGTGGAGAACCATCATCTGCATTTCGATTGTTC |
| | GCTGCAATTCGCATTGTTAGTTGTGTATTTGAGTTATGTGTACTTGGTTTCCAAGCACTTTGGTTCCTTTTTG |
| | CGAGTTTTGGGCAGCGCTGGCTGGTTCCTTTTATAGGAATTAGCTGCACCTTTTGCTTCAAATAAACGCCTG |
| | CTCGTTCACCTGTCTTCCAAAGTTC |
| | AATGCAATGTTTTGTTGCCCAAGTCTTCATTTCTGACTGATGGTGATGTTATGTTCTGTCAGTTCTGTTAATC |
| | ACCTGTTTAATGTGGTAGGCTGATGCCTGTTCTTATTATCAAAGGTTGCTGTGCCGGTACC (SEQ ID |
| | NO: 17) |
| SUS4 | GCGGCCGCTGGTCGTCCCCTTTGGTGCTCGTAGCTTGCTCAACTGTTACTGTGTACCACTTGGTACAAACTG |
| | AACCTTAT |
| | CGCAGGGAAGGACCTTCAGTAACTTAGGTGCGGCAGACGGTAGCTAATAAAATGTGCATATGCGCTCGTT |
| | TGTCTTATGCTGAACTGAACCTTGTGCCTCCCTGGCTATATTGGTTGAACATCTAGGTTTATTATGTACATA |
| | AGGCAGTATGTGATCCACCTGTAGCGTCAGGCTACGGTACC (SEQ ID NO: 18) |

TABLE 11
Primers used for the construction of SUS hairpin constructs, restriction
endonuclease (RE) sites are bold and underlined.

| Primer name | Sequence | RE site | Amplicon |
|---|---|---|---|
| Kpn Intro FW | GGGGTACCACCCGGGTGATGCGGTAACTGAT (SEQ ID NO: 19) | KpnI | Intron |
| Bam Intro RV | GCGGGATCCTCCCGGGCTTCAACCTGCAGA (SEQ ID NO: 20) | Bam HI | sense |
| SUS 1 Pac I FW | CCTTAATTAAAGCCCTCCAGCAAGTGACC (SEQ ID NO: 21) | Pac I | Group 2 |
| SUS 1 Bam HI RV | CGGGATCCCTGAGGCAAACATCGATTCCA (SEQ ID NO: 22) | Bam HI | Antisense |
| SUS 2 Pac I FW | CCTTAATTAATGTGGGAAAGAAGAACCCCAA (SEQ ID NO: 23) | Pac I | Group 1 |
| SUS 2 Bam HI RV | CGGGATCCGGCACAGCAACCTTTGATAATAAGA (SEQ ID NO: | Bam HI | Antisense |
| | 24) | | |
| SUS 3 Pac I FW | CCTTAATTAATGGTTCAATCGAAAGTTTGCTTTAT (SEQ ID NO: | Pac I | Group 3 |
| | 25) | | Antisense |
| SUS 3 Bam HI RV | CGGGATCCGTAGCCTGACGCTACAGGTGG (SEQ ID NO: 26) | Bam HI | |

Construct Preparations and Gene Transfer

The intron, SUS sense and antisense fragments (FIG. 18) were restricted from TOPO2.1 vector, ligated into the NotI and PacI sites of the pShortA1T3 vector from the construct shown in FIG. 19. Based on the positive results from both PCR and restriction endonuclease digestion, the constructs were further confirmed by sequencing of the hairpin structures.

The hairpin construct and selectable marker construct pUbKN were co-precipitated on to tungsten microprojectiles and introduced into sugarcane embryogenic callus, followed by selection for Geneticin resistance and regeneration of transgenic plants, essentially as described previously (Bower et al. 1996 Molecular Breeding 2(3): 239-249).

Sugarcane Growth Conditions and Plants Analysed

Sugarcane cultivar Q117 was used in this experiment which is a current elite commercial variety selected for high sucrose yield. Plants were grown in a containment glasshouse under natural light intensity at 28° C. with watering twice a day. Each plant was grown as a single stalk in a 2 L volume square pot, and fertilized with Osmocote® at 10 g/pot for second month after plantation. Leaves were numbered from one for the TVD, with higher numbers for older leaves. Internodes were numbered according to the leaf attached to the node immediately above.

Plant Growth and Development

These are measured using methods routinely applied in sugarcane under glasshouse or field conditions at various states of the variety selection process. Internode number are counted and biomass measured at harvesting time when transgenic canes are grown in glasshouse. For the transgenic sugarcanes grown in the field, they are monitored carefully for any signs of greater susceptibility or resistance in particular lines to common environmental and biotic stresses. Any apparent changes are followed up by specific tests relevant to the particular stress type.

Sugar Profiles

The inventors' research team has developed a high-throughput analysis (7 min per sample) for separation all sugars in sugarcane extracts, using isocratic HPLC at high pH with pulsed electrochemical detection. This allows the determination of sugar profiles down the stem and other organs such as roots and leaves. Relationships are established between the sugar profiles and the measures of gene expression as well as enzyme activities obtained from selected lines.

SUS Expression Levels

Total RNA was isolated from the internodes of sugarcane lines, and 20 g of total RNA per lane was fractionated by 2.2 M formaldehyde and 1.0% agarose gel electrophoresis, blotted on to Hybond™-N⁺ nylon membrane (Amersham Pharmacia Biotech) and hybridized as described previously (Tsai et al., 1998) using randomly primed ³²P labelled probe. The probe was a PCR product, which was sequenced proved to be a SUS2 fragment but shared conserved regions with SUS1.

RT-qPCR was set up on a 384-well plate and run on sequence detection systems. An Eppendorf epMotion™ 5075 Workstation (Eppendorf North America) was used for dispensing reagent, primers and cDNA. The final condition of the 10 μL reaction solution contained 1×SYBR® Green PCR Master Mix (Applied Biosystems), 200 nM primer (Table 12) set including both forward and reverse primer, 1:25 dilution of cDNA (from 40 μL cDNA synthesis). RT-qPCR program was 95° C. for 10 min, 45 cycles of 95° C. for 15 sec and 59° C. for 1 min, and followed by dissociation analysis as 95° C. for 2 min and 60° C. for 15 sec ramping to 95° C. for 15 sec. Individual reactions were performed in 3 replicates. GAPDH gene was used as an internal control.

TABLE 12
Primers used for RT-qPCR.

| Primer | |
|---|---|
| Name | Primer Sequence |
| SUS 1-4 F | TGGTCCGGCTGAGATCATC (SEQ ID NO: 27) |
| SUS 1-4 R | TCCAGTGGCTCGAATCTGTCTG (SEQ ID NO: 28) |
| SUS 2-4 F | GTGCGGTTTGCCAACAATT (SEQ ID NO: 29) |
| SUS 2-4 R | AAATATCTGCAGCCTTGTCACTGT (SEQ ID NO: 30) |
| SUS 4-2 F | CATAACAGGACTGGTTGAAGCTTT (SEQ ID NO: 31) |
| SUS 4-2 R | CCTTGGACTTCTTGACATCATTGTA (SEQ ID NO: |
| | 32) |
| GAPDH FW | CACGGCCACTGGAAGCA (SEQ ID NO: 33) |
| GAPDH RV | TCCTCAGGGTTCCTGATGCC (SEQ ID NO: 34) |

Crude Enzyme Extraction and Measurement of Activities of SUS Enzymes

Enzymes were extracted by grinding the frozen cells in a chilled mortar using 3 volumes of extraction buffer that contained 0.1M Hepes-KOH buffer (pH 7.5), 10 mMMgCl2, 2 mMEDTA, 2 mM EGTA, 10% glycerol, 5 mMDTT, 2% polyvinyl polypyrrolidone and 1× complete protease inhibitor (Roche, Mannheim, Germany). The homogenate was immediately centrifuged at 10, 000 g for 15 min at 4_C. The supernatant was immediately desalted on a PD-10 column (GE Healthcare, Buckinghamshare, UK) that was pre-equilibrated and eluted using the extraction buffer. This desalted extract was used for enzyme assays. Protein concentration was assayed by the Bradford reaction using a Bio-Rad (Hercules, Calif., USA) kit with bovine serum albumin standards. Activities of SUS breakage activity was calculated based on fructose production, which was measured using a BioLC (Dionex, Sunnyvale, Calif., USA).

### Example 3

### Sugar Content in Ratoon Crops

Sugarcane cultivar Q117 is a current elite commercial variety selected for high sucrose yield. Sugarcane cultivars are highly heterozygous, complex polyploid interspecific hybrids of Saccharum species. They have generally low fertility and are propagated vegetatively for both commercial and experimental purposes. Successive crops of sugarcane that includes a plant crop and a number of ratoon crops (usually three to four). A ratoon crop is the new cane which grows from the stubble left behind after harvesting. This enables the farmers to get three or four crops from these before they have to replant. After the final ratoon, the regrowth will be destroyed by either chemical or physical means.

To test whether the high-sucrose phenotype is maintained in the successive generations of the ratoon crops, one bud section of the stalk from the second generation of the transgenic lines A, B or C was planted in the inverted quadrangular truncated pyramid pot (pot volume, 2 L). With 4 replications, a single stalk was grown in the pot with a density of 30 pots/m². Plants were grown in a containment glasshouse under natural light intensity at 28° C. with watering twice a day and fertilized with Osmocote® at 5 g/month for the first and second months, followed by 10 g/month. Plants were harvested at 10 months old and a single tiller was kept to grow as ratoon stalk in each pot for another 11 months in the same glasshouse conditions. Leaves were numbered from one for the top visual dewlap (TVD), with higher numbers for older leaves. internodes were numbered according to the leaf attached to the node immediately above. The measured growth parameters were the height from the soil surface to the TVD, stalk diameter at the lowest above-ground internode, number of nodes and stalk fresh weight.

For stalk samples, a transverse tissue slice was taken at the mid-point of each designated internode and cut into radial sectors that were proportionately representative of the different stalk tissues by area. Sectors (about 0.15 g FW) were placed on a support screen (PromegaSpin Basket, Madison, Wis.) within a 1.5-mL microfuge tube, liquid nitrogen frozen, thawed on ice and centrifuged at 10 000 g for 15 min at 4° C. to collect the juice. Brix was measured with a pocket refractometer PAL-1 (Atago, Tokyo, Japan) from the extracted juice. Unless stated otherwise, statistical analysis was performed using GraphPad Prism Software (V4.0; San Diego, Calif.).

The results presented in FIG. 20 clearly show that consistent with the first and second generation, Brix values were increased significantly compared to the control in the ratoon crops (FIG. 20a) and there was no negative influence on stalk fresh weight, indicating normal growth and development in the transgenic lines (FIGS. 20b and c).

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

1. A method for increasing the concentration or yield of sucrose or sucrose derivatives in a plant, plant part or plant organ (e.g. plant stem) of a sucrose-accumulating crop plant, the method comprising expressing in a cell (e.g., a plant stem cell) of the plant, plant part or plant organ a polynucleotide that comprises a nucleic acid sequence encoding an expression product that inhibits expression of a SUS2 nucleic acid molecule, or reduces the level or activity a SUS2 polypeptide, to thereby increase the concentration or yield of sucrose or sucrose derivatives in the plant, plant part or plant organ, wherein the SUS2 nucleic acid molecule comprises, consists or consists essentially of a nucleotide sequence selected from the group consisting of:
(a) a nucleotide sequence that encodes the amino acid sequence:
[SEQ ID NO: 2]
MAAKLTRLHSLRERLGATFSSHPNELIALFSRYVNQGKGMLQRHQLLAEF
DALFDSDKEKYAPFEDFLRAAQEAIVLPPWVALAIRPRPGVWDYIRVNVS
ELAVEELSVSEYLAFKEQLVDGNSNSNFVLELDFEPFNASFPRPSMSKSI
GNGVQFLNRHLSSKLFQDKESLYPLLNFLKAHNYKGTTMMLNDRIQSLRG
LQSSLRKAEEYLLSVPQDTPYSEFNHRFQELGLEKGWGDTAKRVLDTLHL
LLDLLEAPDPANLEKFLGTIPMMFNVVILSPHGYFAQSNVLGYPDTGGQV
VYILDQVRALENEMLLRIKQQGLDITPKILIVTRLLPDAVGTTCGQRLEK
VIGTEHTDIIRIPFRNENGILRKWISRFDVWPYLETYTEDVASEIMLEMQ
AKPDLIVGNYSDGNLVATLLAHKLGVTQCTIAHALEKTKYPNSDIYLDKF
DSQYHFSCQFTADLIAMNHTDFIITSTFQEIAGSKDTVGQYESHIAFTLP
GLYRVVHGIDVFDPKFNIVSPGADMSVYYPYTETDKRLTAFHPEIEELIY
SDVENDEHKFVLKDKNKPIIFSMARLDRVKNMTGLVEMYGKNARLRELAN
LVIVAGDHGKESKDREEQAEFKKMYSLIDEYNLKGHIRWISAQMNRVRNA
ELYRYICDTKGAFVQPAFYEAFGLTVIESMTCGLPTIATCHGGPAEIIVD
GVSGLHIDPYHSDKAADILVNFFEKCKADPSYWDKISQGGLQRIYEKYTW
KLYSERLMTLTGVYGFWKYVSNLERRETRRYLEMFYALKYRSLASAVPLS
FD;
(b) a nucleotide sequence that encodes an amino acid sequence that corresponds to SEQ ID NO: 2, for example, one that shares at least 90% (and at least 91% to at least 99% and all integer percentages in between) sequence similarity or sequence identity with the sequence set forth in SEQ ID NO: 2;
(c) the nucleotide sequence:
[SEQ ID NO: 1]
ttgcccgtcagtgagtcgtattacaccgggtggatggcccggccgacgcg
tccgatctgtcccagttctctgttctgttctgtcgacgccattcctgtgc
tctgccgtcccagcgtttgccaagtattgagtgtcattgagccatggctg
ccaagttgactcgcctccacagtcttcgcgaacgccttggtgccaccttc
tcctctcatcccaatgagctgattgcactcttctccaggtatgttaacca
gggcaagggaatgcttcagcgccatcaactgcttgctgagtttgatgccc
tgtttgatagtgacaaggagaagtatgcgcccttcgaagactttcttcgt
gctgctcaggaagcaattgtgctccctccctgggtagcacttgctatcag
gccaaggcctggtgtctgggattacattcgagtgaatgtaagcgagttgg
ctgtggaggagctgagtgtttctgagtacttggcattcaaggaacagctg
gtggatggaaattccaacagcaactttgttcttgagcttgattttgagcc
cttcaatgcctcattccctcgtccttccatgtcaaagtccattggaaatg
gagtgcaattccttaaccgacacctgtcttccaagttgttccaggacaag
gagagcctgtacccattgctgaatttcctcaaagcccataactacaaggg
cacgacgatgatgttgaatgacagaattcagagcctccgtgggctccagt
catcccttagaaaggcagaagagtatctactgagtgtccctcaagacact
ccctactcagagttcaaccataggttccaagagcttggcttggagaaggg
ttggggtgacactgcaaagcgcgtacttgatacactccacttgcttcttg
accttcttgaggcccctgatcctgccaacttggagaagttccttggaact
ataccaatgatgttcaatgttgttatcctgtctcctcatggctactttgc
ccaatccaatgtgcttggataccctgacactggtggtcaggttgtgtaca
ttttggatcaagtccgtgctttggagaatgagatgcttcttaggattaag
cagcaaggccttgacatcaccccgaagatcctcattgttaccaggctgtt
gcctgatgctgttgggactacgtgcggtcagcgtctggagaaggtcattg
gaaccgagcacacagacattattcgtattccattcagaaatgagaatggt
attctccgcaagtggatctctcgttttgatgtctggccatacctggagac
atacactgaggatgttgccagtgaaataatgttagaaatgcaggccaagc
ctgaccttattgttggcaactacagtgatggcaatctagtcgccactctg
ctcgcgcacaagttgggagttactcagtgtaccattgcccacgccttgga
gaaaaccaaatatcccaactcagacatatacttagacaaatttgacagcc
aataccacttctcatgccagttcacagctgaccttattgccatgaatcac
actgatttcatcatcaccagtacattccaagaaatcgcgggaagcaagga
cactgtggggcagtatgagtcccacattgcgttcactcttcctggacttt
accgtgttgtccatggcattgatgtttttgatcccaaattcaacattgtc
tctcctggagcagacatgagtgtttactacccatacactgaaactgacaa
gagactcactgccttccatcctgaaattgaggagctcatctacagtgatg
ttgagaatgatgagcacaagtttgtgttgaaggacaagaacaagccgatc
atcttctcaatggctcgtcttgaccgtgtgaagaacatgacaggcttggt
tgagatgtatggtaagaatgcacgcctgagggaattggcaaaccttgtga
ttgttgctggtgaccatggcaaggaatcgaaggacagggaggagcaggca
gagttcaagaagatgtacagtctcattgatgagtacaacttgaagggcca
tatccggtggatctcagctcagatgaaccgtgtccgcaacgctgagttgt
accgctacatttgtgacacgaagggagcatttgtgcagcctgcattctat
gaagcattcggcctgactgtcattgagtccatgacgtgcggtttgccaac
aattgcaacctgccatggtggccctgctgaaataattgtggacggggtgt
ctggtttgcacattgatccttaccacagtgacaaggctgcagatattttg
gtcaacttctttgagaagtgcaaggcagacccaagctactgggacaagat
ctcacagggtggactgcagagaatttatgagaagtacacctggaagctct
actccgagaggctgatgaccctgactggtgtatacggattctggaagtat
gtgagcaatctggagaggcgtgagactcgccgctaccttgagatgttcta
tgctctgaaataccgtagcctggcaagtgcggttccattgtccttcgatt
agtgtgggaaagaagaaccccaatctggagtagtggagaaccatcatctg
catttcgattgttcgctgcaattcgcattgttagttgtgtatttgagtta
tgtgtacttggtttccaagcactttggttcctttttgcgagttttgggca
gcgctggctggttccttttataggaattagctgcaccttttgcttcaaat
aaacgcctgctcgttcacctgtcttccaaagttcaatgcaatgttttgtt
gcccaagtcttcatttctgactgatggtgatgttatgttctgtcagttct
gttaatcacctgtttaatgtggtaggctgatgcctgttcttattatcaaa
ggttgctgtgcc,
and
[SEQ ID NO: 3]
atggctgccaagttgactcgcctccacagtcttcgcgaacgccttggtgc
caccttctcctctcatcccaatgagctgattgcactcttctccaggtatg
ttaaccagggcaagggaatgcttcagcgccatcaactgcttgctgagttt
gatgccctgtttgatagtgacaaggagaagtatgcgcccttcgaagactt
tcttcgtgctgctcaggaagcaattgtgctccctccctgggtagcacttg
ctatcaggccaaggcctggtgtctgggattacattcgagtgaatgtaagc
gagttggctgtggaggagctgagtgtttctgagtacttggcattcaagga
acagctggtggatggaaattccaacagcaactttgttcttgagcttgatt
ttgagcccttcaatgcctcattccctcgtccttccatgtcaaagtccatt
ggaaatggagtgcaattccttaaccgacacctgtcttccaagttgttcca
ggacaaggagagcctgtacccattgctgaatttcctcaaagcccataact
acaagggcacgacgatgatgttgaatgacagaattcagagcctccgtggg
ctccagtcatcccttagaaaggcagaagagtatctactgagtgtccctca
agacactccctactcagagttcaaccataggttccaagagcttggcttgg
agaagggttggggtgacactgcaaagcgcgtacttgatacactccacttg
cttcttgaccttcttgaggcccctgatcctgccaacttggagaagttcct
tggaactataccaatgatgttcaatgttgttatcctgtctcctcatggct
actttgcccaatccaatgtgcttggataccctgacactggtggtcaggtt
gtgtacattttggatcaagtccgtgctttggagaatgagatgcttcttag
gattaagcagcaaggccttgacatcaccccgaagatcctcattgttacca
ggctgttgcctgatgctgttgggactacgtgcggtcagcgtctggagaag
gtcattggaaccgagcacacagacattattcgtattccattcagaaatga
gaatggtattctccgcaagtggatctctcgttttgatgtctggccatacc
tggagacatacactgaggatgttgccagtgaaataatgttagaaatgcag
gccaagcctgaccttattgttggcaactacagtgatggcaatctagtcgc
cactctgctcgcgcacaagttgggagttactcagtgtaccattgcccacg
ccttggagaaaaccaaatatcccaactcagacatatacttagacaaattt
gacagccaataccacttctcatgccagttcacagctgaccttattgccat
gaatcacactgatttcatcatcaccagtacattccaagaaatcgcgggaa
gcaaggacactgtggggcagtatgagtcccacattgcgttcactcttcct
ggactttaccgtgttgtccatggcattgatgtttttgatcccaaattcaa
cattgtctctcctggagcagacatgagtgtttactacccatacactgaaa
ctgacaagagactcactgccttccatcctgaaattgaggagctcatctac
agtgatgttgagaatgatgagcacaagtttgtgttgaaggacaagaacaa
gccgatcatcttctcaatggctcgtcttgaccgtgtgaagaacatgacag
gcttggttgagatgtatggtaagaatgcacgcctgagggaattggcaaac
cttgtgattgttgctggtgaccatggcaaggaatcgaaggacagggagga
gcaggcagagttcaagaagatgtacagtctcattgatgagtacaacttga
agggccatatccggtggatctcagctcagatgaaccgtgtccgcaacgct
gagttgtaccgctacatttgtgacacgaagggagcatttgtgcagcctgc
attctatgaagcattcggcctgactgtcattgagtccatgacgtgcggtt
tgccaacaattgcaacctgccatggtggccctgctgaaataattgtggac
ggggtgtctggtttgcacattgatccttaccacagtgacaaggctgcaga
tattttggtcaacttctttgagaagtgcaaggcagacccaagctactggg
acaagatctcacagggtggactgcagagaatttatgagaagtacacctgg
aagctctactccgagaggctgatgaccctgactggtgtatacggattctg
gaagtatgtgagcaatctggagaggcgtgagactcgccgctaccttgaga
tgttctatgctctgaaataccgtagcctggcaagtgcggttccattgtcc
ttcgattag;
(d) a nucleotide sequence that corresponds to SEQ ID NO: 1 or 3, or a complement thereof, for example, one that shares at least 90% (and at least 91% to at least 99% and all integer percentages in between) sequence identity with the sequence set forth in SEQ ID NO: 1 or 3, or a complement thereof; or
(e) a nucleotide sequence that hybridizes under at least medium stringency conditions to the sequence set forth in SEQ ID NO: 1 or 3, or a complement thereof,
wherein the nucleotide sequence of (a), (b), (c), (d) or (e) encodes an amino acid sequence having sucrose synthase activity,
wherein the SUS2 polypeptide comprises, consists or consists essentially of an amino acid sequence selected from:
(i) the amino acid sequence set forth in SEQ ID NO: 2;
(ii) an amino acid sequence that corresponds to SEQ ID NO: 2, for example, one that shares at least 90% (and at least 91% to at least 99% and all integer percentages in between) sequence similarity or sequence identity with the sequence set forth in SEQ ID NO: 2;
(iii) an amino acid sequence which is encoded by the nucleotide sequence set forth in any one of SEQ ID NO: 1 or 3;
(iv) an amino acid sequence which is encoded by a nucleotide sequence that corresponds to SEQ ID NO: 1, or a complement thereof, for example, one that shares at least 90% (and at least 91% to at least 99% and all integer percentages in between) sequence identity with the sequence set forth in SEQ ID NO: 1 or 3, or a complement thereof; or
(v) an amino acid sequence which is encoded by a nucleotide sequence that hybridizes under at least medium stringency conditions to the sequence set forth in SEQ ID NO: 1 or 3, or a complement thereof,
wherein the amino acid sequence of (i), (ii), (iii), (iv) or (v) has sucrose synthase activity.

2. A method for increasing the concentration or yield of sucrose or sucrose derivatives in a plant, plant part or plant organ (e.g. plant stem) of a sucrose-accumulating crop plant, the method comprising introducing a nucleic acid construct into the genome of a plant to produce a transformed plant and regenerating therefrom a stably transformed plant, wherein the nucleic acid construct comprises in operable connection: (1) a promoter that is operable in a cell of the sucrose-accumulating crop plant (e.g., a plant stem cell); and (2) a nucleic acid sequence encoding an expression product that inhibits expression of a SUS2 nucleic acid molecule as defined in claim 1, or reduces the level or activity of a SUS2 polypeptide as defined in claim 1.

3. The method of claim 2, wherein the promoter is a plant stem cell-specific promoter or a plant stem cell-preferential promoter.

4. The method of claim 2, wherein the expression product is a SUS2-inhibiting RNA molecule (e.g., siRNA, shRNA, microRNAs, antisense RNA etc.) that inhibits expression of said SUS2 nucleic acid molecule.

5. The method of claim 2, wherein the expression product is an antibody that is immuno-interactive with said SUS2 polypeptide.

6. The method of claim 1, wherein the concentration or yield of sucrose or sucrose derivatives in the plant, plant part or plant organ is increased by at least about 5% (e.g., at least about 6%, 7%, 8%, 9%, 10%, 15% 20%, 25%, 30%, 40%, 50%, 60%, 70%, 80%, 90%) relative to the concentration or yield of sucrose or sucrose derivatives in a control plant, plant part or plant organ that does express the polynucleotide or contain the nucleic acid sequence.

7. The method of claim 2, further comprising selecting a transformed plant that has an increased concentration or yield of sucrose or sucrose derivatives, as compared to a control plant that does not contain the nucleic acid construct.

8. The method of claim 2, wherein the nucleic acid construct is introduced into regenerable plant cells so as to yield transformed plant cells.

9. The method of claim 8, comprising identifying and selecting the transformed plant cells.

10. The method of claim 9, further comprising regenerating differentiated plants from the identified and selected transformed plant cells.

11. The method of claim 10, comprising selecting a transformed plant cell line from the transformed plant cells for the differentiation of a transgenic plant.

12. The method of claim 8, wherein the regenerable cells are regenerable monocotyledonous plant cells.

13. A genetically modified sucrose-accumulating crop plant, plant part or plant organ (e.g., plant stem cells) comprising plant cells (e.g., plant stem cells) having a decreased level of SUS2 compared to that of a control plant, wherein the genetically modified plant, plant part or plant organ has an increased concentration or yield of sucrose or sucrose derivatives relative to a control plant.

14. The genetically modified plant, plant part or plant organ (e.g., plant stem) of claim 13, wherein the sucrose-accumulating crop plant is selected from the group consisting of sugar beet, corn, sugarcane, and sorghum.

15. The genetically modified plant, plant part or plant organ (e.g., plant stem) of claim 13, wherein the sucrose-accumulating crop plant is a C4 plant (e.g., corn, sugarcane, sorghum, etc.).

16. A method of making a genetically modified sucrose-accumulating crop plant having a decreased level of SUS2 compared to that of a control plant, wherein the genetically modified plant displays an increased concentration or yield of sucrose or sucrose derivatives in its storage organs relative to the control plant, the method comprising providing at least one sucrose-accumulating crop plant cell containing a SUS2 gene encoding a functional SUS2 polypeptide; treating the at least one sucrose-accumulating crop plant cell under conditions effective to inactivate the SUS2 gene, thereby yielding at least one genetically modified sucrose-accumulating crop plant cell containing an inactivated SUS2 gene; and propagating the at least one genetically modified sucrose-accumulating crop plant cell into a genetically modified sucrose-accumulating crop plant, wherein the genetically modified sucrose-accumulating crop plant has a decreased level of SUS2 polypeptide compared to that of the control plant and displays an increased concentration or yield of sucrose or sucrose derivatives in plant storage organs relative to the control plant.

17. The method according to claim 16, wherein the treating comprises subjecting the at least one plant cell to a chemical mutagenizing agent under conditions effective to yield at least one mutant plant cell containing an inactive SUS2 gene.

18. The method of claim 16, wherein the sucrose-accumulating crop plant is selected from the group consisting of sugar beet, corn, sugarcane, and sorghum.

19. The method of claim 16, wherein the sucrose-accumulating crop plant is a C4 plant (e.g., corn, sugarcane, sorghum, etc.).

20. A plant breeding method to transfer genetic material of a genetically modified sucrose-accumulating crop plant according to claim 13, the method comprising: (1) crossing a plant containing that genetic material with a sucrose-accumulating crop plant; (2) recovering reproductive material from the progeny of the cross; and (3) growing plants with increased concentration or yield of sucrose or sucrose derivatives from the reproductive material.

21. (canceled)
